(19) United States
(12) Patent Application Publication
(10) Pub. No.: US 2023/0416238 A1
(43) Pub. Date: Dec. 28, 2023

(54) N-[(1,3-BENZOXAZOL-2-YL)-HETEROCYCLE]AMIDE DERIVATIVES FOR THE TREATMENT AND PROPHYLAXIS OF HEPATITIS B VIRUS INFECTION

(71) Applicant: Hoffmann-La Roche Inc., Little Falls, NJ (US)

(72) Inventors: Xianfeng LIN, Shanghai (CN); Hongying YUN, Shanghai (CN); Bo ZHANG, Shanghai (CN); Xiufang ZHENG, Shanghai (CN)

(73) Assignee: Hoffmann-La Roche Inc., Little Falls, NJ (US)

(21) Appl. No.: 18/319,719

(22) Filed: May 18, 2023

Related U.S. Application Data
(63) Continuation of application No. PCT/EP2021/082470, filed on Nov. 22, 2021.

(30) Foreign Application Priority Data
Nov. 23, 2020 (WO) PCT/CN2020/130879

(51) Int. Cl. C07D 413/04; C07D 413/14; C07D 409/14

(52) U.S. Cl. CPC C07D 413/04; C07D 409/14; C07D 413/14

(57) ABSTRACT

The present invention provides novel compounds having the general formula:

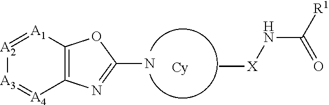

wherein A₁ to A₄, X, Cy and R¹ are as described herein, compositions including the compounds and methods of using the compounds.

### CROSS-REFERENCE TO PRIOR APPLICATIONS

This application is a continuation of International Application No. PCT/EP2021/082470 having an International Filing Date of Nov. 22, 2021 and which claims benefit under 35 U.S.C. § 119 to International Application No. PCT/CN2020/130879 having an International Filing Date of Nov. 23, 2020. The entire contents of both are incorporated herein by reference.

### FIELD OF THE INVENTION

The present invention relates to N-heterocycle amide compounds and their corresponding derivatives of formula (I) useful for therapy and/or prophylaxis of HBV infection in a mammal, and in particular to HBsAg (HBV Surface antigen) and HBeAg (HBV e antigen) inhibitors as well as their manufacture and pharmaceutical compositions containing them.

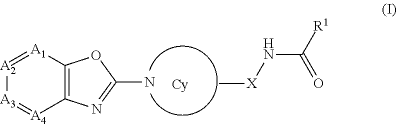

The present invention relates to compounds of formula (I) wherein A₁ to A₄, Cy, X and R¹ are as described below, or a pharmaceutically acceptable salt thereof.

### BACKGROUND OF THE INVENTION

Hepatitis B virus (HBV) is one of the most dangerous human pathogens. A safe and effective vaccine has been available for longer than two decades; however, WHO estimated that approximately 257 million people are chronically infected with HBV. Chronic Hepatitis B (CHB) infection predisposes its host to severe liver disease, including liver cirrhosis and hepatocellular carcinoma, if left untreated. HBV infection is ranked among the top unmet medical need worldwide. The currently approved drugs have contributed to substantial progress in CHB treatment; however, the cure rate remains less than 10%.

The control of viral infection needs an effective immune surveillance. Upon recognition of viral infection, the host innate immune system could respond within minutes to impede viral replication and limits the development of a chronic and persistent infection. The secretion of antiviral cytokines from infected hepatocytes and intra-hepatic immune cells is critically important for the clearance of viral infection. However, chronically infected patients only display a weak immune response due to various escape strategies adopted by the virus to counteract the host cell recognition systems and the subsequent antiviral responses.

Many observations showed that several HBV viral proteins could counteract the initial host cellular response by interfering with the viral recognition signaling system and subsequently the interferon (IFN) antiviral activity. Among these, the excessive secretion of HBV empty subviral particles (SVPs, HBsAg) may contribute to immune tolerant state observed in CHB patients. The persistent exposure to HBsAg and other viral antigens can lead to HBV-specific T-cell functional impairment and depletion (Kondo et al. Journal of Immunology (1993), 150, 4659-4671; Kondo et al. Journal of Medical Virology (2004), 74, 425-433; Fisicaro et al. Gastroenterology, (2010), 138, 682-693;). Moreover, HBsAg has been reported to suppress immune cell functions, including monocytes, dendritic cells (DCs) and natural killer (NK) cells (Op den Brouw et al. Immunology, (2009b), 126, 280-289; Woltman et al. PLoS One, (2011), 6, e15324; Shi et al. J Viral Hepat. (2012), 19, e26-33; Kondo et al. ISRN Gasteroenterology, (2013), Article ID 935295).

HBsAg is an important biomarker for prognosis and treatment response in CHB. However, the achievement of HBsAg loss and seroconversion is rarely achieved in CHB patients. HBsAg loss with or without anti-HBsAg seroconversion remains the ideal clinical treatment endpoints. Current therapies, such as nucleos(t)ide analogues, are effective in supressing HBV DNA, but are not effective in reducing HBsAg level. Nucleos(t)ide analogs, even with prolonged therapy, have demonstrated HBsAg clearance rates comparable to those observed naturally (Janssen et al. Lancet, (2005), 365, 123-129; Marcellin et al. N Engl. J. Med., (2004), 351, 1206-1217; Buster et al. Hepatology, (2007), 46, 388-394). Therefore, there is an urgent need for the development of novel therapeutic agents that could efficiently reduce HBsAg. (Wieland, S. F. & F. V. Chisari. J Virol, (2005), 79, 9369-9380; Kumar et al. J Virol, (2011), 85, 987-995; Woltman et al. PLoS One, (2011), 6, e15324; Op den Brouw et al. Immunology, (2009b), 126, 280-289).

### SUMMARY OF THE INVENTION

Objects of the present invention are novel compounds of formula (I), their manufacture, medicaments based on a compound in accordance with the invention and their production as well as the use of compounds of formula (I) as HBV inhibitors and for the treatment or prophylaxis of HBV infection. The compounds of formula (I) show superior anti-HBV activity. In addition, the compounds of formula (I) also show good safety and good PK profiles.

The present invention relates to a compound of formula (I)

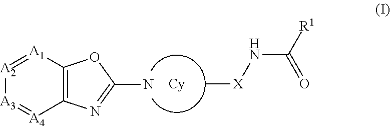

wherein
R¹ is C₁₋₆alkyl, C₁₋₆alkylamino, (C₁₋₆alkyl)₂amino, C₁₋₆alkoxy, C₃₋₇cycloalkyl, phenyl, heterocyclyl or hetercyclylC₁₋₆alkyl; wherein C₁₋₆alkyl, C₁₋₆alkylamino, (C₁₋₆alkyl)₂amino, C₁₋₆alkoxy, C₃₋₇cycloalkyl, phenyl, heterocyclyl and hetercyclylC₁₋₆alkyl are unsubstituted or substituted by one or two or three substituents independently selected from aminocarbonyl, haloC₁₋₆alkyl, C₃₋₇cycloalkylsulfonyl, C₁₋₆alkylsulfonyl, aminosulfonyl, C₁₋₆alkylphenylsulfonyl, OH, halogen, C₁₋₆alkyl, C₁₋₆alkoxy, C₁₋₆alkoxyC₁₋₆alkoxy, C₁₋₆ alkylsulfonimidoyl, C₁₋₆alkylsulfonylC₁₋₆alkyl, C₃₋₇cycloalkylC₁₋₆alkylsulfonimidoyl, C₃₋₇ cycloalkylaminosulfonyl, haloC₃₋₇cycloalkylaminosulfonyl, C₁₋₆alkylsulfinyl, C₃₋₇cycloalkylC₁₋₆alkylsulfonyl, C₃₋₇cycloalkylC₁₋₆alkylsulfinyl, C₃₋₇cycloalkylcarbonylaminosulfonyl and C₃₋₇cycloalkylsulfonimidoyl;
Cy is N-containing heterocyclyl; wherein N-containing heterocyclyl is unsubstituted or substituted by C₁₋₆alkyl;
X is a bond or CH₂;
A₁ is N or CR²; wherein R² is H, halogen, C₁₋₆alkyl, or C₁₋₆alkoxy;
A₂ is N or CR³; wherein R³ is H, halogen, C₁₋₆alkyl, or C₁₋₆alkoxy;
A₃ is N or CR⁴; wherein R⁴ is H, halogen, C₁₋₆alkyl, or C₁₋₆alkoxy;
A₄ is N or CR⁵; wherein R⁵ is H, halogen, C₁₋₆alkyl, or C₁₋₆alkoxy;
or a pharmaceutically acceptable salt thereof.

### DETAILED DESCRIPTION OF THE INVENTION

### Definitions

As used herein, the term “C₁₋₆alkyl” alone or in combination signifies a saturated, linear- or branched chain alkyl group containing 1 to 6, particularly 2 to 6 or 1 to 4 carbon atoms, for example methyl, ethyl, propyl, isopropyl, butyl, isobutyl, tert-butyl and the like. Particular “C₁₋₆alkyl” groups are methyl, ethyl, propyl, isopropyl, isobutyl and tert-butyl.

The term “C₁₋₆alkoxy” alone or in combination signifies a group C₁₋₆alkyl-O—, wherein the “C₁₋₆alkyl” is as defined above; for example methoxy, ethoxy, propoxy, iso-propoxy, n-butoxy, iso-butoxy, 2-butoxy, tert-butoxy, pentoxy, hexyloxy and the like. Particular “C₁₋₆alkoxy” groups are methoxy and ethoxy and propoxy.

The term “halogen” denotes fluoro, chloro, bromo, or iodo.

The term “haloC₁₋₆alkyl” denotes an alkyl group wherein at least one of the hydrogen atoms of the alkyl group is replaced by same or different halogen atoms, particularly fluoro atoms. Examples of haloC₁₋₆alkyl include monochloro-, difluoro- or trifluoro-methyl, -ethyl or -propyl, for example difluoromethyl and trifluoromethyl.

The term “C₃₋₇cycloalkyl” denotes to a saturated carbon ring containing from 3 to 7 carbon atoms, particularly from 3 to 6 carbon atoms, for example, cyclopropyl, cyclobutyl, cyclopentyl, cyclohexyl, cycloheptyl and the like. Particular “C₃₋₇cycloalkyl” group is cyclopropyl, cyclobutyl or cyclopentyl.

“heterocyclyl” refers to any mono-, bi-, tricyclic or spiro, saturated or unsaturated, aromatic (heteroaryl) or non-aromatic (e.g., heterocycloalkyl), ring system, having 3 to 20 ring atoms, where the ring atoms are carbon, and at least one atom in the ring or ring system is a heteroatom selected from nitrogen, sulfur or oxygen. If any ring atom of a cyclic system is a heteroatom, that system is a heterocyclyl, regardless of the point of attachment of the cyclic system to the rest of the molecule. In one example, heterocyclyl includes 3-11 ring atoms (“members”) and includes monocycles, bicycles, tricycles and spiro ring systems, wherein the ring atoms are carbon, where at least one atom in the ring or ring system is a heteroatom selected from nitrogen, sulfur or oxygen. In one example, heterocyclyl includes 3- to 7-membered monocycles having 1, 2, 3 or 4 heteroatoms selected from nitrogen, sulfur or oxygen. In another example, heterocyclyl includes 4-, 5- or 6-membered monocycles having 1, 2, 3 or 4 heteroatoms selected from nitrogen, sulfur or oxygen. In one example, heterocyclyl includes 8- to 12-membered bicycles having 1, 2, 3, 4, 5 or 6 heteroatoms selected from nitrogen, sulfur or oxygen. In another example, heterocyclyl includes 9- or 10-membered bicycles having 1, 2, 3, 4, 5 or 6 heteroatoms selected from nitrogen, sulfur or oxygen. Exemplary heterocyclyls are furyl, pyridyl, pyrrolidinyl, piperidyl, oxetanyl, tetrahydrofuranyl, tetrahydrothiopyranyl, azetidinylmethyl, pyrrolidinylmethyl, piperidylmethyl, tetrahydrofuranylmethyl, 1-oxothianyl, 1,1-dioxothietanyl, 1,1-dioxothiolanyl, 1,1-dioxothianyl, 1,1-dioxothietanylmethyl and 2,2-dioxo-2-λ⁶-thiaspiro[3.3]heptanyl. Heterocyclyl may be optionally substituted by halogen, OH, SH, cyano, NH₂, NHCH₃, N(CH₃)₂, NO₂, N₃, C(O)CH₃, COOH, CO₂CH₃, C₁₋₆alkyl, C₁₋₆alkoxy, oxo, haloC₁₋₆alkyl, phenyl or heterocyclyl.

The term “carbonyl” alone or in combination refers to the group —C(O)—.

The term “sulfanyl” alone or in combination refers to the group —S—.

The term “sulfinyl” alone or in combination refers to the group —S(O)—.

The term “sulfonyl” alone or in combination refers to the group —S(O)₂—.

The term “sulfonimidoyl” alone or in combination refers to the group —S(O)(NH)—, whose formula is

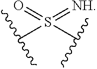 [SMILES: *S(*)(=N)=O]

The term “bond” refers to a chemical bond between two atoms, or two moieties when the atoms joined by the bond are considered to be part of larger substructure. In one aspect, when a group described herein is a bond, the referenced group is absent thereby allowing a bond to be formed between the remaining identified groups.

As used herein, the wavy line “ [symbol] ” that intersects a bond in a chemical structure refers to the point of attachment of the bond to which the wavy bond intersects in the chemical structure fragment to the remainder of a molecule or structural formula.

The compounds according to the present invention may exist in the form of their pharmaceutically acceptable salts. The term “pharmaceutically acceptable salt” refers to conventional acid-addition salts or base-addition salts that retain the biological effectiveness and properties of the compounds of formula (I) and are formed from suitable non-toxic organic or inorganic acids or organic or inorganic bases. Acid-addition salts include for example those derived from inorganic acids such as hydrochloric acid, hydrobromic acid, hydroiodic acid, sulfuric acid, sulfamic acid, phosphoric acid and nitric acid, and those derived from organic acids such as p-toluenesulfonic acid, salicylic acid, methanesulfonic acid, oxalic acid, succinic acid, citric acid, malic acid, lactic acid, fumaric acid, and the like. Base-addition salts include those derived from ammonium, potassium, sodium and, quaternary ammonium hydroxides, such as for example, tetramethyl ammonium hydroxide. The chemical modification of a pharmaceutical compound into a salt is a technique well known to pharmaceutical chemists in order to obtain improved physical and chemical stability, hygroscopicity, flowability and solubility of compounds. It is for example described in Bastin R. J., et al., Organic Process Research & Development 2000, 4, 427-435. Particular are the sodium salts of the compounds of formula (I).

### HBV Inhibitors

The present invention provides (i) a compound having the general formula (I):

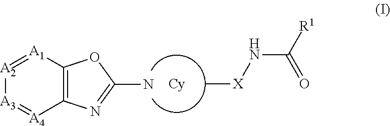

wherein
R¹ is C₁₋₆alkyl, C₁₋₆alkylamino, (C₁₋₆alkyl)₂amino, C₁₋₆alkoxy, C₃₋₇cycloalkyl, phenyl, heterocyclyl or hetercyclylC₁₋₆alkyl; wherein C₁₋₆alkyl, C₁₋₆alkylamino, (C₁₋₆alkyl)₂amino, C₁₋₆alkoxy, C₃₋₇cycloalkyl, phenyl, heterocyclyl and hetercyclylC₁₋₆alkyl are unsubstituted or substituted by one or two or three substituents independently selected from aminocarbonyl, haloC₁₋₆alkyl, C₃₋₇cycloalkylsulfonyl, C₁₋₆alkylsulfonyl, aminosulfonyl, C₁₋₆alkylphenylsulfonyl, OH, halogen, C₁₋₆alkyl, C₁₋₆alkoxy, C₁₋₆alkoxyC₁₋₆alkoxy, C₁₋₆alkylsulfonimidoyl, C₁₋₆alkylsulfonylC₁₋₆alkyl, C₃₋₇cycloalkylC₁₋₆alkylsulfonimidoyl, C₃₋₇cycloalkylaminosulfonyl, haloC₃₋₇cycloalkylaminosulfonyl, C₁₋₆alkylsulfinyl, C₃₋₇cycloalkylC₁₋₆alkylsulfonyl, C₃₋₇cycloalkylC₁₋₆alkylsulfinyl, C₃₋₇cycloalkylcarbonylaminosulfonyl and C₃₋₇cycloalkylsulfonimidoyl;
Cy is N-containing heterocyclyl; wherein N-containing heterocyclyl is unsubstituted or substituted by C₁₋₆alkyl;
X is a bond or CH₂;
A₁ is N or CR²; wherein R² is H, halogen, C₁₋₆alkyl, or C₁₋₆alkoxy;
A₂ is N or CR³; wherein R³ is H, halogen, C₁₋₆alkyl, or C₁₋₆alkoxy;
A₃ is N or CR⁴; wherein R⁴ is H, halogen, C₁₋₆alkyl, or C₁₋₆alkoxy;
A₄ is N or CR⁵; wherein R⁵ is H, halogen, C₁₋₆alkyl, or C₁₋₆alkoxy;
or a pharmaceutically acceptable salt thereof.

A further embodiment of the present invention is (ii) a compound of formula (I) according to (i) wherein
R¹ is C₁₋₆alkyl, C₁₋₆alkylamino, (C₁₋₆alkyl)₂amino, C₁₋₆alkoxy, C₃₋₇cycloalkyl, phenyl, furyl, pyridyl, pyrrolidinyl, piperidyl, oxetanyl, tetrahydrofuranyl, tetrahydrothiopyranyl, azetidinylmethyl, pyrrolidinylmethyl, piperidylmethyl, tetrahydrofuranylmethyl, 1-oxothianyl, 1,1-dioxothietanyl, 1,1-dioxothiolanyl, 1,1-dioxothianyl, 1,1-dioxothietanylmethyl or 2,2-dioxo-2-λ⁶-thiaspiro[3.3]heptanyl; wherein C₁₋₆ alkyl, C₃₋₇cycloalkyl, phenyl, furyl, pyridyl, pyrrolidinyl, piperidyl, tetrahydrothiopyranyl, azetidinylmethyl, pyrrolidinylmethyl, piperidylmethyl, 1-oxothianyl, 1,1-dioxothiolanyl and 1,1-dioxothianyl are unsubstituted or substituted by one or two or three substituents independently selected from aminocarbonyl, haloC₁₋₆alkyl, C₃₋₇cycloalkylsulfonyl, C₁₋₆alkylsulfonyl, aminosulfonyl, C₁₋₆alkylphenylsulfonyl, OH, halogen, C₁₋₆alkyl, C₁₋₆alkoxy, C₁₋₆alkoxyC₁₋₆alkoxy, C₁₋₆alkylsulfonimidoyl, C₁₋₆alkylsulfonylC₁₋₆alkyl, C₃₋₇cycloalkylC₁₋₆alkylsulfonimidoyl, C₃₋₇cycloalkylaminosulfonyl, haloC₃₋₇cycloalkylaminosulfonyl, C₁₋₆alkylsulfinyl, C₃₋₇cycloalkylC₁₋₆alkylsulfonyl, C₃₋₇cycloalkylC₁₋₆alkylsulfinyl, C₃₋₇cycloalkylcarbonylaminosulfonyl and C₃₋₇cycloalkylsulfonimidoyl;
Cy is selected from the group consisting of

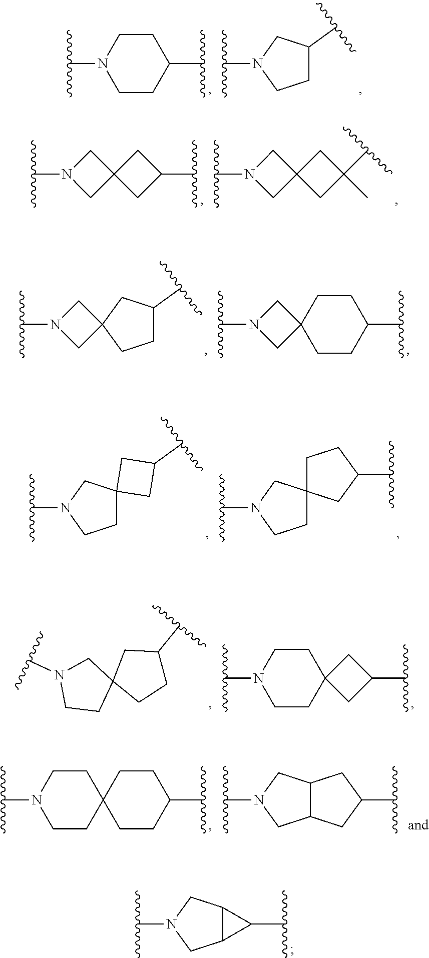

X is a bond or CH₂;
A₁ is N or CH;
A₂ is CR³; wherein R³ is H or halogen;
A₃ is CR⁴; wherein R⁴ is H or halogen;
A₄ is CH;
or a pharmaceutically acceptable salt thereof.

A further embodiment of the present invention is (iii) a compound of formula (I) according to any one of (i)-(ii), wherein
R¹ is methyl, ethyl, propyl, isopropyl, tert-butyl, isobutyl, dimethylamino, ethylamino, ethoxy, isobutoxy, cyclopropyl, cyclobutyl, cyclopentyl, cyclohextyl, phenyl, furyl, pyridyl, pyrrolidinyl, piperidyl, oxetanyl, tetrahydrofuranyl, tetrahydrothiopyranyl, azetidinylmethyl, pyrrolidinylmethyl, piperidylmethyl, tetrahydrofuranylmethyl, 1-oxothianyl, 1,1-dioxothietanyl, 1,1-dioxothiolanyl, 1,1-dioxothianyl, 1,1-dioxothietanylmethyl or 2,2-dioxo-2-λ⁶-thiaspiro[3.3]heptanyl; wherein methyl, ethyl, propyl, isopropyl, cyclopropyl, cyclobutyl, cyclopentyl, cyclohextyl, phenyl, furyl, pyridyl, pyrrolidinyl, piperidyl, tetrahydrothiopyranyl, azetidinylmethyl, pyrrolidinylmethyl, piperidylmethyl, 1-oxothianyl, 1,1-dioxothiolanyl and 1,1-dioxothianyl are unsubstituted or substituted by one or two or three substituents independently selected from aminocarbonyl, CF₃, cyclopropylsulfonyl, methylsulfonyl, aminosulfonyl, methylphenylsulfonyl, OH, F, methyl, methoxy, methoxyethoxy, methylsulfonimidoyl, isopropoxy, ethoxy, isobutylsulfonimidoyl, methylsulfonylmethyl, ethylsulfonyl, isobutylsulfonyl, cyclopropylmethylsulfonimidoyl, cyclopropylaminosulfonyl, difluorocyclobutylaminosulfonyl, cyclopropylmethylsulfinyl, isobutylsulfinyl, methylsulfinyl, cyclopropylmethylsulfonyl, ethylsulfinyl, cyclobutylcarbonylaminosulfonyl, ethylsulfonimidoyl and cyclopropylsulfonimidoyl;
Cy is selected from the group consisting of

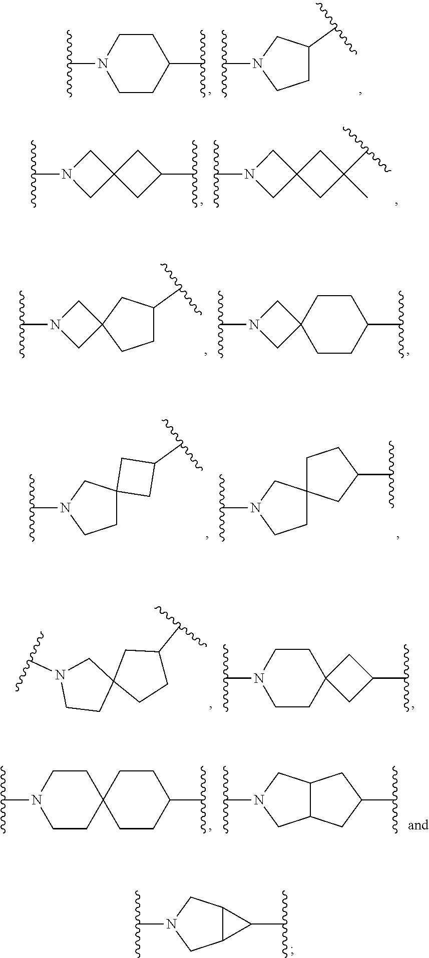

X is a bond or CH₂;
A₁ is N or CH;
A₂ is CR³; wherein R³ is H or Cl;
A₃ is CR⁴; wherein R⁴ is H or Cl;
A₄ is CH;
or a pharmaceutically acceptable salt thereof.

A further embodiment of the present invention is (iv) a compound of formula (I) according to any one of (i)-(iii), or a pharmaceutically acceptable salt thereof, wherein A₁ is CH.

A further embodiment of the present invention is (v) a compound of formula (I) according to any one of (i)-(iv), or a pharmaceutically acceptable salt thereof, wherein A₁, A₂, A₃ and A₄ are not CH simultaneously.

A further embodiment of the present invention is (vi) a compound of formula (I) according to any one of (i)-(v), or a pharmaceutically acceptable salt thereof, wherein Cy is selected from the group consisting of

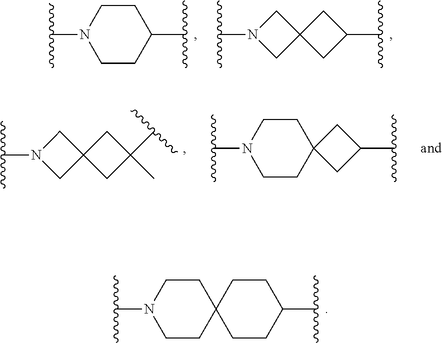

A further embodiment of the present invention is (vii) a compound of formula (I) according to any one of (i)-(vi), or a pharmaceutically acceptable salt thereof, wherein when Cy is

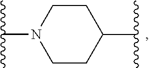 [SMILES: *C1CCN(*)CC1]

### X is CH₂.

A further embodiment of the present invention is (viii) a compound of formula (I) according to any one of (i)-(vii), or a pharmaceutically acceptable salt thereof, wherein when Cy is

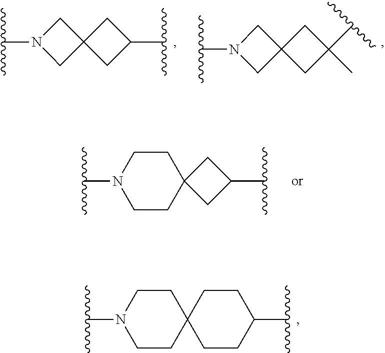

X is a bond.

A further embodiment of the present invention is (ix) a compound of formula (I) according to any one of (i)-(viii), or a pharmaceutically acceptable salt thereof, wherein R¹ is C₁₋₆alkyl, furyl, pyridyl, tetrahydrofuranyl, tetrahydrothiopyranyl, tetrahydrofuranylmethyl, 1,1-dioxothietanyl, 1,1-dioxothiolanyl, 1,1-dioxothianyl or 2,2-dioxo-2-λ⁶-thiaspiro[3.3]heptanyl; wherein C₁₋₆alkyl, furyl, pyridyl, tetrahydrothiopyranyl and 1,1-dioxothiolanyl are unsubstituted or substituted one time independently selected from haloC₁₋₆alkyl, C₃₋₇cycloalkylsulfonyl, C₁₋₆alkylsulfonyl, C₁₋₆alkyl, C₁₋₆alkoxy, C₁₋₆alkylsulfonimidoyl, C₃₋₇cycloalkylC₁₋₆alkylsulfonimidoyl, C₁₋₆alkylsulfinyl, C₃₋₇cycloalkylC₁₋₆alkylsulfonyl, C₃₋₇cycloalkylC₁₋₆alkylsulfinyl, and C₃₋₇cycloalkylsulfonimidoyl.

A further embodiment of the present invention is (x) a compound of formula (I) according to any one of (i)-(ix), or a pharmaceutically acceptable salt thereof, wherein R¹ is ethyl, tert-butyl, furyl, pyridyl, tetrahydrofuranyl, tetrahydrothiopyranyl, tetrahydrofuranylmethyl, 1,1-dioxothietanyl, 1,1-dioxothiolanyl, 1,1-dioxothianyl or 2,2-dioxo-2-λ⁶-thiaspiro[3.3]heptanyl; wherein ethyl, furyl, pyridyl, tetrahydrothiopyranyl and 1,1-dioxothiolanyl are unsubstituted or substituted one time independently selected from methylsulfonyl, methoxy, isobutylsulfonimidoyl, ethylsulfonyl, isobutylsulfonyl, CF₃, cyclopropylmethylsulfonimidoyl, cyclopropylmethylsulfinyl, isobutylsulfinyl, cyclopropylmethylsulfonyl, cyclopropylsulfonyl, methyl, ethylsulfinyl and cyclopropylsulfonimidoyl.

A further embodiment of the present invention is (xi) a compound of formula (I) according to any one of (i)-(x), or a pharmaceutically acceptable salt thereof, wherein
R¹ is C₁₋₆alkyl, furyl, pyridyl, tetrahydrofuranyl, tetrahydrothiopyranyl, tetrahydrofuranylmethyl, 1,1-dioxothietanyl, 1,1-dioxothiolanyl, 1,1-dioxothianyl or 2,2-dioxo-2-λ⁶-thiaspiro[3.3]heptanyl; wherein C₁₋₆alkyl, furyl, pyridyl, tetrahydrothiopyranyl and 1,1-dioxothiolanyl are unsubstituted or substituted one time independently selected from haloC₁₋₆alkyl, C₃₋₇cycloalkylsulfonyl, C₁₋₆alkylsulfonyl, C₁₋₆alkyl, C₁₋₆alkoxy, C₁₋₆alkylsulfonimidoyl, C₃₋₇cycloalkylC₁₋₆alkylsulfonimidoyl, C₁₋₆alkylsulfinyl, C₃₋₇cycloalkylC₁₋₆alkylsulfonyl, C₃₋₇cycloalkylC₁₋₆alkylsulfinyl, and C₃₋₇cycloalkylsulfonimidoyl;
Cy is selected from the group consisting of

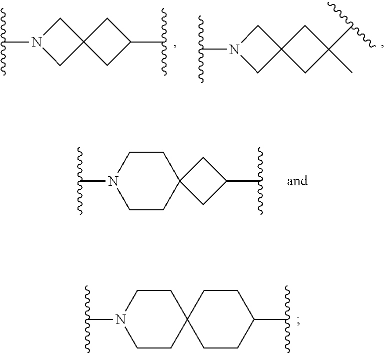

X is a bond;
A₁ is CH;
A₂ is CR³; wherein R³ is H or halogen;
A₃ is CR⁴; wherein R⁴ is H or halogen;
A₄ is CH;
wherein with the proviso that A₁, A₂, A₃ and A₄ are not CH simultaneously.

A further embodiment of the present invention is (xii) a compound of formula (I) according to any one of (i)-(xi), or a pharmaceutically acceptable salt thereof, wherein
R¹ is ethyl, tert-butyl, furyl, pyridyl, tetrahydrofuranyl, tetrahydrothiopyranyl, tetrahydrofuranylmethyl, 1,1-dioxothietanyl, 1,1-dioxothiolanyl, 1,1-dioxothianyl or 2,2-dioxo-2-λ⁶-thiaspiro[3.3]heptanyl; wherein ethyl, furyl, pyridyl, tetrahydrothiopyranyl and 1,1-dioxothiolanyl are unsubstituted or substituted one time independently selected from methylsulfonyl, methoxy, isobutylsulfonimidoyl, ethylsulfonyl, isobutylsulfonyl, CF₃, cyclopropylmethylsulfonimidoyl, cyclopropylmethylsulfinyl, isobutylsulfinyl, cyclopropylmethylsulfonyl, cyclopropylsulfonyl, methyl, ethylsulfinyl and cyclopropylsulfonimidoyl;
Cy is selected from the group consisting of

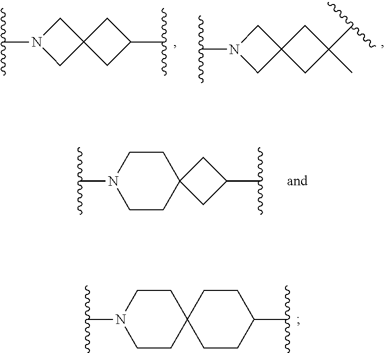

X is a bond;
A₁ is CH;
A₂ is CR³; wherein R³ is H or Cl;
A₃ is CR⁴; wherein R⁴ is H or Cl;
A₄ is CH;
wherein with the proviso that A₁, A₂, A₃ and A₄ are not CH simultaneously.

In another embodiment (xiii) of the present invention, particular compounds of the present invention are selected from:
N2-[[1-(5-chloro-1,3-benzoxazol-2-yl)-4-piperidyl]methyl]furan-2,5-dicarboxamide;
N-[[1-(5-chloro-1,3-benzoxazol-2-yl)-4-piperidyl]methyl]-6-(trifluoromethyl)pyridine-3-carboxamide;
N-[[1-(5-chloro-1,3-benzoxazol-2-yl)-4-piperidyl]methyl]-2-cyclopropylsulfonyl-pyridine-4-carboxamide;
N-[[1-(5-chloro-1,3-benzoxazol-2-yl)-4-piperidyl]methyl]-3-methylsulfonyl-benzamide;
N-[[1-(5-chloro-1,3-benzoxazol-2-yl)-4-piperidyl]methyl]-2-methylsulfonyl-pyridine-4-carboxamide;
N4-[[1-(5-chloro-1,3-benzoxazol-2-yl)-4-piperidyl]methyl]pyridine-2,4-dicarboxamide;
N-[[1-(5-chloro-1,3-benzoxazol-2-yl)-4-piperidyl]methyl]-5-(trifluoromethyl)furan-2-carboxamide;
N-[[1-(5-chloro-1,3-benzoxazol-2-yl)-4-piperidyl]methyl]-5-sulfamoyl-furan-2-carboxamide;
N-[[1-(5-chloro-1,3-benzoxazol-2-yl)-4-piperidyl]methyl]-5-methylsulfonyl-pyridine-3-carboxamide;
N-[[1-(5-chloro-1,3-benzoxazol-2-yl)-4-piperidyl]methyl]-1,1-dioxo-thietane-3-carboxamide;
N-[[1-(5-chloro-1,3-benzoxazol-2-yl)pyrrolidin-3-yl]methyl]-2-cyclopropylsulfonyl-pyridine-4-carboxamide;
N-[1-(5-chloro-1,3-benzoxazol-2-yl)-4-piperidyl]-5-(trifluoromethyl)furan-2-carboxamide;
N-[[1-(5-chloro-1,3-benzoxazol-2-yl)-4-piperidyl]methyl]-1-methylsulfonyl-pyrrolidine-3-carboxamide;
N-[[1-(5-chloro-1,3-benzoxazol-2-yl)-4-piperidyl]methyl]-1-(p-tolylsulfonyl)pyrrolidine-3-carboxamide;
N-[[1-(5-chloro-1,3-benzoxazol-2-yl)-4-piperidyl]methyl]-1-methylsulfonyl-piperidine-4-carboxamide;
N-[[1-(5-chloro-1,3-benzoxazol-2-yl)-4-piperidyl]methyl]-1-(p-tolylsulfonyl)piperidine-4-carboxamide;
N-[[1-(5-chloro-1,3-benzoxazol-2-yl)-4-piperidyl]methyl]-2-(1-methylsulfonylazetidin-3-yl)acetamide;
N-[[1-(5-chloro-1,3-benzoxazol-2-yl)-4-piperidyl]methyl]-2-[1-(p-tolylsulfonyl)pyrrolidin-3-yl]acetamide;
N-[[1-(5-chloro-1,3-benzoxazol-2-yl)-4-piperidyl]methyl]-2-(1-methylsulfonyl-4-piperidyl)acetamide;
N-[[1-(5-chloro-1,3-benzoxazol-2-yl)-4-piperidyl]methyl]-2-[1-(p-tolylsulfonyl)-4-piperidyl]acetamide;
ethyl N-[2-(5-chloro-1,3-benzoxazol-2-yl)-2-azaspiro[3.3]heptan-6-yl]carbamate;
isobutyl N-[2-(5-chloro-1,3-benzoxazol-2-yl)-2-azaspiro[3.3]heptan-6-yl]carbamate;
3-[2-(5-chloro-1,3-benzoxazol-2-yl)-2-azaspiro[3.3]heptan-6-yl]-1,1-dimethyl-urea;
1-[2-(5-chloro-1,3-benzoxazol-2-yl)-2-azaspiro[3.3]heptan-6-yl]-3-ethyl-urea;
N-[2-(5-chloro-1,3-benzoxazol-2-yl)-2-azaspiro[3.3]heptan-6-yl]-4,4,4-trifluoro-butanamide;
N-[2-(5-chloro-1,3-benzoxazol-2-yl)-2-azaspiro[3.3]heptan-6-yl]-1,1-dioxo-thiolane-3-carboxamide;
N-[2-(5-chloro-1,3-benzoxazol-2-yl)-2-azaspiro[3.3]heptan-6-yl]-2,2-dimethyl-propanamide;
N-[2-(5-chloro-1,3-benzoxazol-2-yl)-2-azaspiro[3.3]heptan-6-yl]-3-methyl-butanamide;
N-[2-(5-chloro-1,3-benzoxazol-2-yl)-2-azaspiro[3.3]heptan-6-yl]-2-hydroxy-2-methyl-propanamide;
N-[2-(5-chloro-1,3-benzoxazol-2-yl)-2-azaspiro[3.3]heptan-6-yl]-2,2-difluoro-cyclopropanecarboxamide;
N-[2-(5-chloro-1,3-benzoxazol-2-yl)-2-azaspiro[3.3]heptan-6-yl]-3,3-difluoro-cyclobutanecarboxamide;
N-[2-(5-chloro-1,3-benzoxazol-2-yl)-2-azaspiro[3.3]heptan-6-yl]-3,3-difluoro-cyclopentanecarboxamide;
N-[2-(5-chloro-1,3-benzoxazol-2-yl)-2-azaspiro[3.3]heptan-6-yl]-3-methyl-1,1-dioxo-thiolane-3-carboxamide;
N-[2-(5-chloro-1,3-benzoxazol-2-yl)-2-azaspiro[3.3]heptan-6-yl]-1,1-dioxo-thietane-3-carboxamide;
N-[2-(5-chloro-1,3-benzoxazol-2-yl)-2-azaspiro[3.3]heptan-6-yl]-2-(1,1-dioxothietan-3-yl)acetamide;
N-[2-(5-chloro-1,3-benzoxazol-2-yl)-2-azaspiro[3.3]heptan-6-yl]-1,1-dioxo-thiane-4-carboxamide;
N-[2-(6-chloro-1,3-benzoxazol-2-yl)-2-azaspiro[3.3]heptan-6-yl]-2-cyclopropylsulfonyl-pyridine-4-carboxamide;
N-[2-(6-chloro-1,3-benzoxazol-2-yl)-2-azaspiro[3.3]heptan-6-yl]-2-methoxy-pyridine-4-carboxamide;
N-[2-(5-chloro-1,3-benzoxazol-2-yl)-2-azaspiro[3.3]heptan-6-yl]-2-(2-methoxyethoxy)pyridine-4-carboxamide;
N-[2-(5-chloro-1,3-benzoxazol-2-yl)-2-azaspiro[3.3]heptan-6-yl]-5-(methylsulfonimidoyl)furan-2-carboxamide;
N-[2-(6-chloro-1,3-benzoxazol-2-yl)-2-azaspiro[3.3]heptan-6-yl]-2-isopropoxy-pyridine-4-carboxamide;
N-[2-(5-chloro-1,3-benzoxazol-2-yl)-2-azaspiro[3.3]heptan-6-yl]-5-(trifluoromethyl)furan-2-carboxamide;
N-[2-(5-chloro-1,3-benzoxazol-2-yl)-2-azaspiro[3.3]heptan-6-yl]-2-methoxy-pyridine-4-carboxamide;
N-[2-(5-chloro-1,3-benzoxazol-2-yl)-2-azaspiro[3.3]heptan-6-yl]-5-cyclopropylsulfonyl-furan-2-carboxamide;
N-[2-(5-chloro-1,3-benzoxazol-2-yl)-2-azaspiro[3.3]heptan-6-yl]-2-methyl-pyridine-4-carboxamide;
N-[2-(5-chloro-1,3-benzoxazol-2-yl)-2-azaspiro[3.3]heptan-6-yl]-6-methoxy-pyridine-3-carboxamide;
N-[2-(5-chloro-1,3-benzoxazol-2-yl)-2-azaspiro[3.3]heptan-6-yl]-5-[(S)-methylsulfonimidoyl]furan-2-carboxamide;
N-[2-(6-chlorooxazolo[5,4-b]pyridin-2-yl)-2-azaspiro[3.3]heptan-6-yl]-5-methylsulfonyl-furan-2-carboxamide;
N-[2-(5-chloro-1,3-benzoxazol-2-yl)-2-azaspiro[3.3]heptan-6-yl]-2-ethoxy-pyridine-4-carboxamide;
N-[2-(5-chloro-1,3-benzoxazol-2-yl)-2-azaspiro[3.3]heptan-6-yl]-5-(isobutylsulfonimidoyl)furan-2-carboxamide;
N-[2-(5-chloro-1,3-benzoxazol-2-yl)-2-azaspiro[3.3]heptan-6-yl]-5-(methylsulfonylmethyl)furan-2-carboxamide;
N-[2-(5-chloro-1,3-benzoxazol-2-yl)-2-azaspiro[3.3]heptan-6-yl]-2-methylsulfonyl-pyridine-4-carboxamide;
N-[2-(5-chloro-1,3-benzoxazol-2-yl)-2-azaspiro[3.3]heptan-6-yl]-2-ethylsulfonyl-pyridine-4-carboxamide;
N-[2-(5-chloro-1,3-benzoxazol-2-yl)-2-azaspiro[3.3]heptan-6-yl]-5-isobutylsulfonyl-furan-2-carboxamide;
N-[2-(5-chloro-1,3-benzoxazol-2-yl)-2-azaspiro[3.3]heptan-6-yl]-2-(trifluoromethyl)pyridine-4-carboxamide;
N-[2-(5-chloro-1,3-benzoxazol-2-yl)-2-azaspiro[3.3]heptan-6-yl]-5-methylsulfonyl-furan-2-carboxamide;
N-[2-(5-chloro-1,3-benzoxazol-2-yl)-2-azaspiro[3.3]heptan-6-yl]-5-(cyclopropylmethylsulfonimidoyl)furan-2-carboxamide;
N-[2-(5-chloro-1,3-benzoxazol-2-yl)-2-azaspiro[3.3]heptan-6-yl]-5-ethylsulfonyl-furan-2-carboxamide;
N-[2-(5-chloro-1,3-benzoxazol-2-yl)-2-azaspiro[3.3]heptan-6-yl]-5-(cyclopropylsulfamoyl)furan-2-carboxamide;
N-[2-(5-chloro-1,3-benzoxazol-2-yl)-2-azaspiro[3.3]heptan-6-yl]-5-[(3,3-difluorocyclobutyl)sulfamoyl]furan-2-carboxamide;
N-[2-(5-chloro-1,3-benzoxazol-2-yl)-2-azaspiro[3.3]heptan-6-yl]-5-(cyclopropylmethylsulfinyl)furan-2-carboxamide;
N-[2-(5-chloro-1,3-benzoxazol-2-yl)-2-azaspiro[3.3]heptan-6-yl]-5-isobutylsulfinyl-furan-2-carboxamide;
N-[2-(5-chloro-1,3-benzoxazol-2-yl)-2-azaspiro[3.3]heptan-6-yl]-5-[(R)-methylsulfonimidoyl]furan-2-carboxamide;
N-[2-(5-chloro-1,3-benzoxazol-2-yl)-2-azaspiro[3.3]heptan-6-yl]-5-methylsulfinyl-furan-2-carboxamide;
N-[2-(5-chloro-1,3-benzoxazol-2-yl)-2-azaspiro[3.3]heptan-6-yl]-5-(cyclopropylmethylsulfonyl)furan-2-carboxamide;
N-[2-(5-chloro-1,3-benzoxazol-2-yl)-2-azaspiro[3.3]heptan-6-yl]-2-cyclopropylsulfonyl-pyridine-4-carboxamide;
N-[2-(5-chloro-1,3-benzoxazol-2-yl)-6-methyl-2-azaspiro[3.3]heptan-6-yl]-5-methylsulfonyl-furan-2-carboxamide;
N-[2-(5-chloro-1,3-benzoxazol-2-yl)-6-methyl-2-azaspiro[3.3]heptan-6-yl]-2-(trifluoromethyl)pyridine-4-carboxamide;
N-[2-(5-chloro-1,3-benzoxazol-2-yl)-2-azaspiro[3.4]octan-6-yl]-5-(trifluoromethyl)furan-2-carboxamide;
N-[2-(5-chloro-1,3-benzoxazol-2-yl)-2-azaspiro[3.5]nonan-7-yl]-5-(trifluoromethyl)furan-2-carboxamide;
N-[6-(5-chloro-1,3-benzoxazol-2-yl)-6-azaspiro[3.4]octan-2-yl]-1,1-dioxo-thiolane-3-carboxamide;
N-[6-(5-chloro-1,3-benzoxazol-2-yl)-6-azaspiro[3.4]octan-2-yl]-5-(trifluoromethyl)furan-2-carboxamide;
N-[6-(6-chloro-1,3-benzoxazol-2-yl)-6-azaspiro[3.4]octan-2-yl]-1,1-dioxo-thiolane-3-carboxamide;
N-[2-(5-chloro-1,3-benzoxazol-2-yl)-2-azaspiro[4.4]nonan-8-yl]-1,1-dioxo-thiolane-3-carboxamide;
N-[2-(5-chloro-1,3-benzoxazol-2-yl)-2-azaspiro[4.4]nonan-8-yl]-5-(trifluoromethyl)furan-2-carboxamide;
N-[7-(5-chloro-1,3-benzoxazol-2-yl)-7-azaspiro[3.5]nonan-2-yl]-1,1-dioxo-thiolane-3-carboxamide;
N-[7-(5-chloro-1,3-benzoxazol-2-yl)-7-azaspiro[3.5]nonan-2-yl]-5-(trifluoromethyl)furan-2-carboxamide;
N-[3-(5-chloro-1,3-benzoxazol-2-yl)-3-azaspiro[5.5]undecan-9-yl]-2,2-dimethyl-propanamide;
N-[3-(5-chloro-1,3-benzoxazol-2-yl)-3-azaspiro[5.5]undecan-9-yl]-2-methyl-propanamide;
N-[3-(5-chloro-1,3-benzoxazol-2-yl)-3-azaspiro[5.5]undecan-9-yl]-3-methyl-butanamide;
N-[3-(5-chloro-1,3-benzoxazol-2-yl)-3-azaspiro[5.5]undecan-9-yl]-2-methylsulfonyl-acetamide;
N-[3-(5-chloro-1,3-benzoxazol-2-yl)-3-azaspiro[5.5]undecan-9-yl]-3-sulfamoyl-propanamide;
N-[3-(5-chloro-1,3-benzoxazol-2-yl)-3-azaspiro[5.5]undecan-9-yl]-3-methylsulfonyl-propanamide;
N-[3-(5-chloro-1,3-benzoxazol-2-yl)-3-azaspiro[5.5]undecan-9-yl]-2-tetrahydrofuran-3-yl-acetamide;
N-[3-(5-chloro-1,3-benzoxazol-2-yl)-3-azaspiro[5.5]undecan-9-yl]oxetane-3-carboxamide;
N-[3-(5-chloro-1,3-benzoxazol-2-yl)-3-azaspiro[5.5]undecan-9-yl]tetrahydrofuran-3-carboxamide;
N-[3-(5-chloro-1,3-benzoxazol-2-yl)-3-azaspiro[5.5]undecan-9-yl]-4-methyl-tetrahydrothiopyran-4-carboxamide;
N-[3-(5-chloro-1,3-benzoxazol-2-yl)-3-azaspiro[5.5]undecan-9-yl]-1,1-dioxo-thietane-3-carboxamide;
N-[3-(6-chloro-1,3-benzoxazol-2-yl)-3-azaspiro[5.5]undecan-9-yl]-1,1-dioxo-thietane-3-carboxamide;
N-[3-(5-chloro-1,3-benzoxazol-2-yl)-3-azaspiro[5.5]undecan-9-yl]-1,1-dioxo-thiolane-3-carboxamide;
(3S)—N-[3-(5-chloro-1,3-benzoxazol-2-yl)-3-azaspiro[5.5]undecan-9-yl]-1,1-dioxo-thiolane-3-carboxamide;
(3R)—N-[3-(5-chloro-1,3-benzoxazol-2-yl)-3-azaspiro[5.5]undecan-9-yl]-1,1-dioxo-thiolane-3-carboxamide;
N-[3-(5-chloro-1,3-benzoxazol-2-yl)-3-azaspiro[5.5]undecan-9-yl]-3-methyl-1,1-dioxo-thiolane-3-carboxamide;
N-[3-(6-chloro-1,3-benzoxazol-2-yl)-3-azaspiro[5.5]undecan-9-yl]-1,1-dioxo-thiolane-3-carboxamide;
N-[3-(5-chloro-1,3-benzoxazol-2-yl)-3-azaspiro[5.5]undecan-9-yl]-2,2-dioxo-2-λ⁶ -thiaspiro[3.3]heptane-6-carboxamide;
N-[3-(5-chloro-1,3-benzoxazol-2-yl)-3-azaspiro[5.5]undecan-9-yl]-1,1-dioxo-thiane-4-carboxamide;
N-[3-(6-chloro-1,3-benzoxazol-2-yl)-3-azaspiro[5.5]undecan-9-yl]-1,1-dioxo-thiane-4-carboxamide;
N-[3-(5-chloro-1,3-benzoxazol-2-yl)-3-azaspiro[5.5]undecan-9-yl]-4-methyl-1-oxo-thiane-4-carboxamide;
N-[3-(5-chloro-1,3-benzoxazol-2-yl)-3-azaspiro[5.5]undecan-9-yl]-4-methyl-1,1-dioxo-thiane-4-carboxamide;
N-[3-(5-chloro-1,3-benzoxazol-2-yl)-3-azaspiro[5.5]undecan-9-yl]-1,1-dioxo-thiane-3-carboxamide;
N-[3-(5-chloro-1,3-benzoxazol-2-yl)-3-azaspiro[5.5]undecan-9-yl]-3,3-difluoro-cyclopentanecarboxamide;
N-[3-(5-chloro-1,3-benzoxazol-2-yl)-3-azaspiro[5.5]undecan-9-yl]-4,4-difluoro-cyclohexanecarboxamide;
N-[3-(5-chloro-1,3-benzoxazol-2-yl)-3-azaspiro[5.5]undecan-9-yl]-5-ethylsulfinyl-furan-2-carboxamide;
N-[3-(5-chloro-1,3-benzoxazol-2-yl)-3-azaspiro[5.5]undecan-9-yl]-5-(cyclopropylmethylsulfinyl)furan-2-carboxamide;
N-[3-(5-chloro-1,3-benzoxazol-2-yl)-3-azaspiro[5.5]undecan-9-yl]-2-(trifluoromethyl)pyridine-4-carboxamide;
N-[3-(5-chloro-1,3-benzoxazol-2-yl)-3-azaspiro[5.5]undecan-9-yl]-2-methoxy-pyridine-4-carboxamide;
N-[3-(5-chloro-1,3-benzoxazol-2-yl)-3-azaspiro[5.5]undecan-9-yl]-2-methyl-pyridine-4-carboxamide;
N-[3-(5-chloro-1,3-benzoxazol-2-yl)-3-azaspiro[5.5]undecan-9-yl]-5-sulfamoyl-furan-2-carboxamide;
N-[3-(5-chloro-1,3-benzoxazol-2-yl)-3-azaspiro[5.5]undecan-9-yl]-5-(methylsulfonimidoyl)furan-2-carboxamide;
N-[3-(1,3-benzoxazol-2-yl)-3-azaspiro[5.5]undecan-9-yl]-2-cyclopropylsulfonyl-pyridine-4-carboxamide;
N-[3-(5-chloro-1,3-benzoxazol-2-yl)-3-azaspiro[5.5]undecan-9-yl]-5-(cyclobutanecarbonylsulfamoyl)furan-2-carboxamide;
N-[3-(5-chloro-1,3-benzoxazol-2-yl)-3-azaspiro[5.5]undecan-9-yl]-5-(methylsulfonylmethyl)furan-2-carboxamide;
N-[3-(5-chloro-1,3-benzoxazol-2-yl)-3-azaspiro[5.5]undecan-9-yl]-5-methylsulfonyl-furan-2-carboxamide;
N-[3-(5-chloro-1,3-benzoxazol-2-yl)-3-azaspiro[5.5]undecan-9-yl]-5-(ethylsulfonimidoyl)furan-2-carboxamide;
N-[3-(5-chloro-1,3-benzoxazol-2-yl)-3-azaspiro[5.5]undecan-9-yl]-5-(trifluoromethyl)furan-2-carboxamide;
N-[3-(5-chloro-1,3-benzoxazol-2-yl)-3-azaspiro[5.5]undecan-9-yl]-5-methylsulfinyl-furan-2-carboxamide;
N-[3-(5-chloro-1,3-benzoxazol-2-yl)-3-azaspiro[5.5]undecan-9-yl]-5-ethylsulfonyl-furan-2-carboxamide;
N-[3-(5-chloro-1,3-benzoxazol-2-yl)-3-azaspiro[5.5]undecan-9-yl]-2-cyclopropylsulfonyl-pyridine-4-carboxamide;
N-[3-(5-chloro-1,3-benzoxazol-2-yl)-3-azaspiro[5.5]undecan-9-yl]-5-(cyclopropylmethylsulfonimidoyl)furan-2-carboxamide;
N-[3-(5-chloro-1,3-benzoxazol-2-yl)-3-azaspiro[5.5]undecan-9-yl]-5-(cyclopropylsulfonimidoyl)furan-2-carboxamide;
N-[3-(5-chloro-1,3-benzoxazol-2-yl)-3-azaspiro[5.5]undecan-9-yl]-5-(cyclopropylmethylsulfonyl)furan-2-carboxamide;
N-[3-(5-chloro-1,3-benzoxazol-2-yl)-3-azaspiro[5.5]undecan-9-yl]-5-cyclopropylsulfonyl-furan-2-carboxamide;
N-[2-(5-chloro-1,3-benzoxazol-2-yl)-3,3a,4,5,6,6a-hexahydro-1H-cyclopenta[c]pyrrol-5-yl]-5-(trifluoromethyl)furan-2-carboxamide;
N-[2-(5-chloro-1,3-benzoxazol-2-yl)-3,3a,4,5,6,6a-hexahydro-1H-cyclopenta[c]pyrrol-5-yl]-1,1-dioxo-thiolane-3-carboxamide; and
N-[3-(5-chloro-1,3-benzoxazol-2-yl)-3-azabicyclo[3.1.0]hexan-6-yl]-5-(trifluoromethyl)furan-2-carboxamide;
or a pharmaceutically acceptable salt thereof.

In another embodiment (xiv) of the present invention, particular compounds of the present invention are selected from:
N-[[1-(5-chloro-1,3-benzoxazol-2-yl)-4-piperidyl]methyl]-5-methylsulfonyl-pyridine-3-carboxamide;
N-[2-(5-chloro-1,3-benzoxazol-2-yl)-2-azaspiro[3.3]heptan-6-yl]-2-methoxy-pyridine-4-carboxamide;
N-[2-(5-chloro-1,3-benzoxazol-2-yl)-2-azaspiro[3.3]heptan-6-yl]-5-(isobutylsulfonimidoyl)furan-2-carboxamide;
N-[2-(5-chloro-1,3-benzoxazol-2-yl)-2-azaspiro[3.3]heptan-6-yl]-2-methylsulfonyl-pyridine-4-carboxamide;
N-[2-(5-chloro-1,3-benzoxazol-2-yl)-2-azaspiro[3.3]heptan-6-yl]-2-ethylsulfonyl-pyridine-4-carboxamide;
N-[2-(5-chloro-1,3-benzoxazol-2-yl)-2-azaspiro[3.3]heptan-6-yl]-5-isobutylsulfonyl-furan-2-carboxamide;
N-[2-(5-chloro-1,3-benzoxazol-2-yl)-2-azaspiro[3.3]heptan-6-yl]-2-(trifluoromethyl)pyridine-4-carboxamide;
N-[2-(5-chloro-1,3-benzoxazol-2-yl)-2-azaspiro[3.3]heptan-6-yl]-5-methylsulfonyl-furan-2-carboxamide;
N-[2-(5-chloro-1,3-benzoxazol-2-yl)-2-azaspiro[3.3]heptan-6-yl]-5-(cyclopropylmethylsulfonimidoyl)furan-2-carboxamide;
N-[2-(5-chloro-1,3-benzoxazol-2-yl)-2-azaspiro[3.3]heptan-6-yl]-5-ethylsulfonyl-furan-2-carboxamide;
N-[2-(5-chloro-1,3-benzoxazol-2-yl)-2-azaspiro[3.3]heptan-6-yl]-5-(cyclopropylmethylsulfinyl)furan-2-carboxamide;
N-[2-(5-chloro-1,3-benzoxazol-2-yl)-2-azaspiro[3.3]heptan-6-yl]-5-isobutylsulfinyl-furan-2-carboxamide;
N-[2-(5-chloro-1,3-benzoxazol-2-yl)-2-azaspiro[3.3]heptan-6-yl]-5-(cyclopropylmethylsulfonyl)furan-2-carboxamide;
N-[2-(5-chloro-1,3-benzoxazol-2-yl)-2-azaspiro[3.3]heptan-6-yl]-2-cyclopropylsulfonyl-pyridine-4-carboxamide;
N-[2-(5-chloro-1,3-benzoxazol-2-yl)-6-methyl-2-azaspiro[3.3]heptan-6-yl]-5-methylsulfonyl-furan-2-carboxamide;
N-[7-(5-chloro-1,3-benzoxazol-2-yl)-7-azaspiro[3.5]nonan-2-yl]-1,1-dioxo-thiolane-3-carboxamide;
N-[3-(5-chloro-1,3-benzoxazol-2-yl)-3-azaspiro[5.5]undecan-9-yl]-2,2-dimethyl-propanamide;
N-[3-(5-chloro-1,3-benzoxazol-2-yl)-3-azaspiro[5.5]undecan-9-yl]-3-methylsulfonyl-propanamide;
N-[3-(5-chloro-1,3-benzoxazol-2-yl)-3-azaspiro[5.5]undecan-9-yl]-2-tetrahydrofuran-3-yl-acetamide;
N-[3-(5-chloro-1,3-benzoxazol-2-yl)-3-azaspiro[5.5]undecan-9-yl]tetrahydrofuran-3-carboxamide;
N-[3-(5-chloro-1,3-benzoxazol-2-yl)-3-azaspiro[5.5]undecan-9-yl]-4-methyl-tetrahydrothiopyran-4-carboxamide;
N-[3-(5-chloro-1,3-benzoxazol-2-yl)-3-azaspiro[5.5]undecan-9-yl]-1,1-dioxo-thietane-3-carboxamide;
N-[3-(5-chloro-1,3-benzoxazol-2-yl)-3-azaspiro[5.5]undecan-9-yl]-1,1-dioxo-thiolane-3-carboxamide;
(3S)—N-[3-(5-chloro-1,3-benzoxazol-2-yl)-3-azaspiro[5.5]undecan-9-yl]-1,1-dioxo-thiolane-3-carboxamide;
(3R)—N-[3-(5-chloro-1,3-benzoxazol-2-yl)-3-azaspiro[5.5]undecan-9-yl]-1,1-dioxo-thiolane-3-carboxamide;
N-[3-(5-chloro-1,3-benzoxazol-2-yl)-3-azaspiro[5.5]undecan-9-yl]-3-methyl-1,1-dioxo-thiolane-3-carboxamide;
N-[3-(6-chloro-1,3-benzoxazol-2-yl)-3-azaspiro[5.5]undecan-9-yl]-1,1-dioxo-thiolane-3-carboxamide;
N-[3-(5-chloro-1,3-benzoxazol-2-yl)-3-azaspiro[5.5]undecan-9-yl]-2,2-dioxo-2-λ⁶-thiaspiro[3.3]heptane-6-carboxamide;
N-[3-(5-chloro-1,3-benzoxazol-2-yl)-3-azaspiro[5.5]undecan-9-yl]-1,1-dioxo-thiane-4-carboxamide;
N-[3-(5-chloro-1,3-benzoxazol-2-yl)-3-azaspiro[5.5]undecan-9-yl]-1,1-dioxo-thiane-3-carboxamide;
N-[3-(5-chloro-1,3-benzoxazol-2-yl)-3-azaspiro[5.5]undecan-9-yl]-5-ethylsulfinyl-furan-2-carboxamide;
N-[3-(5-chloro-1,3-benzoxazol-2-yl)-3-azaspiro[5.5]undecan-9-yl]-5-(cyclopropylmethylsulfinyl)furan-2-carboxamide;
N-[3-(5-chloro-1,3-benzoxazol-2-yl)-3-azaspiro[5.5]undecan-9-yl]-2-(trifluoromethyl)pyridine-4-carboxamide;
N-[3-(5-chloro-1,3-benzoxazol-2-yl)-3-azaspiro[5.5]undecan-9-yl]-2-methoxy-pyridine-4-carboxamide;
N-[3-(5-chloro-1,3-benzoxazol-2-yl)-3-azaspiro[5.5]undecan-9-yl]-2-methyl-pyridine-4-carboxamide;
N-[3-(5-chloro-1,3-benzoxazol-2-yl)-3-azaspiro[5.5]undecan-9-yl]-2-cyclopropylsulfonyl-pyridine-4-carboxamide;
N-[3-(5-chloro-1,3-benzoxazol-2-yl)-3-azaspiro[5.5]undecan-9-yl]-5-(cyclopropylmethylsulfonimidoyl)furan-2-carboxamide;
N-[3-(5-chloro-1,3-benzoxazol-2-yl)-3-azaspiro[5.5]undecan-9-yl]-5-(cyclopropylsulfonimidoyl)furan-2-carboxamide;
N-[3-(5-chloro-1,3-benzoxazol-2-yl)-3-azaspiro[5.5]undecan-9-yl]-5-(cyclopropylmethylsulfonyl)furan-2-carboxamide; and
N-[3-(5-chloro-1,3-benzoxazol-2-yl)-3-azaspiro[5.5]undecan-9-yl]-5-cyclopropylsulfonyl-furan-2-carboxamide;
or a pharmaceutically acceptable salt thereof.

### SYNTHESIS

The compounds of the present invention can be prepared by any conventional means. Suitable processes for synthesizing these compounds as well as their starting materials are provided in the schemes below and in the examples. All substituents, in particular, A₁ to A₄, Cy, X and R¹ are as defined above unless otherwise indicated. Furthermore, and unless explicitly otherwise stated, all reactions, reaction conditions, abbreviations and symbols have the meanings well known to a person of ordinary skill in organic chemistry.

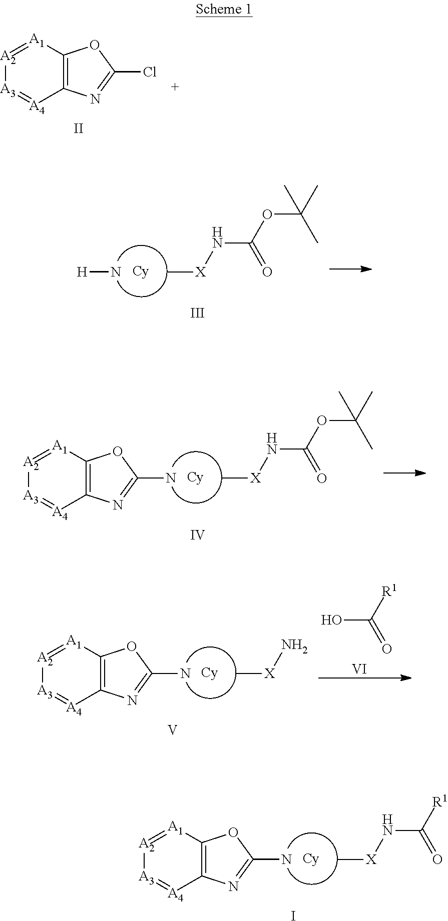

The compound of formula IV can be prepared by coupling of a compound of formula II with a compound of formula III in a suitable base, such as K₂CO₃ or Et₃N, in a suitable solvent, such as acetonitrile, THF or CH₂Cl₂. The compound of formula V can be deprotected with a suitable acid, such as HCl or TFA, in a suitable solvent, such as CH₂Cl₂ or dioxane. The compound of formula I can be prepared by coupling of a compound of formula V with a compound of formula VI in the presence of a suitable coupling reagent, such as propylphosphonic anhydride and O-(7-azabenzotriazol-1-yl)-N,N,N′,N′-tetramethyluronium hexafluorophosphate, and a suitable base, such as triethylamine.

This invention also relates to a process for the preparation of a compound of formula (I) comprising the following step:
(a) Coupling of a compound of formula (V),

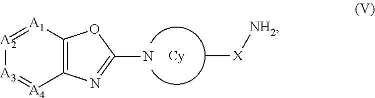

with a compound of formula (VI),

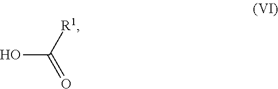

in the presence of a coupling reagent and a base;
The coupling reagent in step (a), can be for example, propylphosphonic anhydride or O-(7-azabenzotriazol-1-yl)-N,N,N′,N′-tetramethyluronium hexafluorophosphate;
The base in step (a), can be for example, triethylamine.

A compound of formula (I) when manufactured according to the above process is also an object of the invention.

The compound of this invention also shows good safety and PK profile.

### Pharmaceutical Compositions and Administration

The invention also relates to a compound of formula (I) for use as therapeutically active substance. Another embodiment provides pharmaceutical compositions or medicaments containing the compounds of the invention and a therapeutically inert carrier, diluent or excipient, as well as methods of using the compounds of the invention to prepare such compositions and medicaments. In one example, compounds of formula (I) may be formulated by mixing at ambient temperature at the appropriate pH, and at the desired degree of purity, with physiologically acceptable carriers, i.e., carriers that are non-toxic to recipients at the dosages and concentrations employed into a galenical administration form. The pH of the formulation depends mainly on the particular use and the concentration of compound, but preferably ranges anywhere from about 3 to about 8. In one example, a compound of formula (I) is formulated in an acetate buffer, at pH 5. In another embodiment, the compounds of formula (I) are sterile. The compound may be stored, for example, as a solid or amorphous composition, as a lyophilized formulation or as an aqueous solution.

Compositions are formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The “effective amount” of the compound to be administered will be governed by such considerations, and is the minimum amount necessary to reduction of HBsAg and HBeAg in HBV patients. For example, such amount may be below the amount that is toxic to normal cells, or the mammal as a whole.

In one example, the pharmaceutically effective amount of the compound of the invention administered parenterally per dose will be in the range of about 0.1 to 100 mg/kg, alternatively about 0.1 to 50 mg/kg of patient body weight per day, with the typical initial range of compound used being 0.3 to 15 mg/kg/day. In another embodiment, oral unit dosage forms, such as tablets and capsules, preferably contain from about 25 to about 1000 mg of the compound of the invention.

The compounds of the invention may be administered by any suitable means, including oral, topical (including buccal and sublingual), rectal, vaginal, transdermal, parenteral, subcutaneous, intraperitoneal, intrapulmonary, intradermal, intrathecal and epidural and intranasal, and, if desired for local treatment, intralesional administration. Parenteral infusions include intramuscular, intravenous, intraarterial, intraperitoneal, or subcutaneous administration.

The compounds of the present invention may be administered in any convenient administrative form, e.g., tablets, powders, capsules, solutions, dispersions, suspensions, syrups, sprays, suppositories, gels, emulsions, patches, etc. Such compositions may contain components conventional in pharmaceutical preparations, e.g., diluents, carriers, pH modifiers, sweeteners, bulking agents, and further active agents.

A typical formulation is prepared by mixing a compound of the present invention and a carrier or excipient. Suitable carriers and excipients are well known to those skilled in the art and are described in detail in, e.g., Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug (i.e., a compound of the present invention or pharmaceutical composition thereof) or aid in the manufacturing of the pharmaceutical product (i.e., medicament).

An example of a suitable oral dosage form is a tablet containing about 25 to 500 mg of the compound of the invention compounded with about 90 to 30 mg anhydrous lactose, about 5 to 40 mg sodium croscarmellose, about 5 to 30 mg polyvinylpyrrolidone (PVP) K30, and about 1 to 10 mg magnesium stearate. The powdered ingredients are first mixed together and then mixed with a solution of the PVP. The resulting composition can be dried, granulated, mixed with the magnesium stearate and compressed to tablet form using conventional equipment. An example of an aerosol formulation can be prepared by dissolving the compound, for example 5 to 400 mg, of the invention in a suitable buffer solution, e.g. a phosphate buffer, adding a tonicifier, e.g. a salt such sodium chloride, if desired. The solution may be filtered, e.g., using a 0.2 micron filter, to remove impurities and contaminants.

An embodiment, therefore, includes a pharmaceutical composition comprising a compound of formula (I), or a pharmaceutically acceptable salt thereof.

In a further embodiment includes a pharmaceutical composition comprising a compound of formula (I), or a pharmaceutically acceptable salt thereof, together with a pharmaceutically acceptable carrier or excipient.

Another embodiment includes a pharmaceutical composition comprising a compound of formula (I), or a pharmaceutically acceptable salt thereof for use in the treatment of HBV infection.

### Indications and Methods of Treatment

The compounds of the invention have anti-HBV activity. Accordingly, the compounds of the invention are useful for the treatment or prophylaxis of HBV infection.

The invention also relates to the use of a compound of formula (I) for the inhibition of HBeAg.

The invention further relates to the use of a compound of formula (I) for the inhibition of HBsAg.

The invention relates to the use of a compound of formula (I) for the inhibition of HBV DNA.

The invention relates to the use of a compound of formula (I) for use in the treatment or prophylaxis of HBV infection.

The use of a compound of formula (I) for the preparation of medicaments useful in the treatment or prophylaxis diseases that are related to HBV infection is an object of the invention.

The invention relates in particular to the use of a compound of formula (I) for the preparation of a medicament for the treatment or prophylaxis of HBV infection.

Another embodiment includes a method for the treatment or prophylaxis of HBV infection, which method comprises administering an effective amount of a compound of formula (I), or a pharmaceutically acceptable salt thereof.

The invention relates in particular to a compound of formula (I) for use in the treatment or prophylaxis of HBV infection.

### EXAMPLES

The invention will be more fully understood by reference to the following examples. They should not, however, be construed as limiting the scope of the invention.

Abbreviations used herein are as follows:
ACN: acetonitrile
CDCl₃: deuterated chloroform
CD₃OD: deuterated methanol
DMF: dimethylformamide
DMSO-d₆: deuterated dimethylsulfoxide
EtOAc/EA: ethyl acetate
HATU: 2-(3H-[1,2,3]triazolo[4,5-b]pyridin-3-yl)-1,1,3,3-tetramethyl-isouronium hexafluorophosphate(V)
HPLC: high performance liquid chromatography
h: hour
IC₅₀: the half maximal inhibitory concentration
LC-MS: liquid chromatography-mass spectrometry
MeOH: methanol
m-CPBA: 3-chloroperoxybenzoic acid
MHz: megahertz
min: minute
mL: milliliter
mmol: millimole
MS (ESI): mass spectroscopy (electron spray ionization)
NMR: nuclear magnetic resonance
obsd. observed
PE petroleum ether
TEA: triethylamine
TFA: trifluoroacetic acid
THF: tetrahydrofuran
TLC: thin layer chromatography
δ: chemical shift
r.t.: room temperature

### General Experimental Conditions

Intermediates and final compounds were purified by flash chromatography using one of the following instruments: i) Biotage SP1 system and the Quad 12/25 Cartridge module. ii) ISCO combi-flash chromatography instrument. Silica gel Brand and pore size: i) KP-SIL 60 Å, particle size: 40-60 μm; ii) CAS registry NO: Silica Gel: 63231-67-4, particle size: 47-60 micron silica gel; iii) ZCX from Qingdao Haiyang Chemical Co., Ltd, pore: 200-300 or 300-400.

Intermediates and final compounds were purified by preparative HPLC on reversed phase column using X Bridge™ Perp C₁₈ (5 μm, OBD™ 30×100 mm) column or SunFire™ Perp C₁₈ (5 μm, OBD™ 30×100 mm) column.

LC/MS spectra were obtained using an Acquity Ultra Performance LC—3100 Mass Detector or Acquity Ultra Performance LC—SQ Detector. Standard LC/MS conditions were as follows (running time 3 minutes):
Acidic condition: A: 0.1% formic acid in H₂O; B: 0.1% formic acid in acetonitrile;
Basic condition: A: 0.05% NH₃·H₂O in H₂O; B: acetonitrile;
Neutral condition: A: H₂O; B: acetonitrile.

Mass spectra (MS): generally, only ions which indicate the parent mass are reported, and unless otherwise stated the mass ion quoted is the positive mass ion (M+H)⁺.

The microwave assisted reactions were carried out in a Biotage Initiator Sixty or CEM Discover.

NMR Spectra were obtained using Bruker Avance 400 MHz.

All reactions involving air-sensitive reagents were performed under an argon atmosphere. Reagents were used as received from commercial suppliers without further purification unless otherwise noted.

### PREPARATIVE EXAMPLES

The invention will be more fully understood by reference to the following examples. They should not, however, be construed as limiting the scope of the invention.

### Intermediate Int-1

### [1-(5-Chloro-1,3-benzoxazol-2-yl)-4-piperidyl]methanamine 2,2,2-trifluoroacetic acid salt

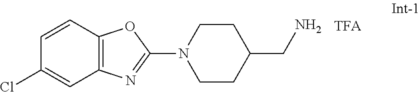 [SMILES: NCC1CCN(c2nc3cc(Cl)ccc3o2)CC1]

The title compound was prepared according to the following scheme:

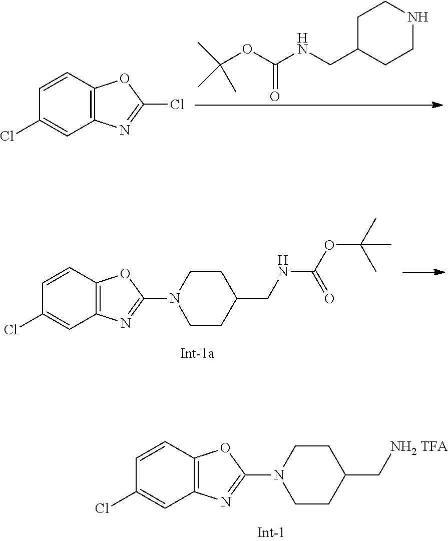

### Step 1: Preparation of tert-butyl N-[[1-(5-chloro-1,3-benzoxazol-2-yl)-4-piperidyl]methyl]carbamate (Int-1a)

2,5-Dichlorobenzo[d]oxazole (1.93 g, 10.3 mmol) was added to a solution of TEA (4.72 g, 6.5 mL, 46.7 mmol) and tert-butyl (piperidin-4-ylmethyl)carbamate (2 g, 9.33 mmol) in CH₂Cl₂ (10 mL) at r.t. Then the mixture was stirred under refluxing overnight. LC-MS detection indicated that the starting materials were consumed. The mixture was concentrated and the residue was purified by silica gel column to afford tert-butyl N-[[1-(5-chloro-1,3-benzoxazol-2-yl)-4-piperidyl]methyl]carbamate (3.36 g). MS obsd. (ESI⁺) [(M+H)⁺]: 366.1.

### Step 2: Preparation of [1-(5-chloro-1,3-benzoxazol-2-yl)-4-piperidyl]methanamine 2,2,2-trifluoroacetic acid salt (Int-1)

2,2,2-Trifluoroacetic acid (20.9 g, 184 mmol) was added to a solution of tert-butyl ((1-(5-chlorobenzo[d]oxazol-2-yl)piperidin-4-yl)methyl)carbamate (3.36 g, 9.18 mmol) in CH₂Cl₂ (50 mL) at r.t. Then the mixture was stirred at r.t. overnight. LC-MS detection indicated that the starting materials were consumed. The mixture was concentrated to afford [1-(5-chloro-1,3-benzoxazol-2-yl)-4-piperidyl]methanamine 2,2,2-trifluoroacetic acid salt (3.60 g). MS obsd. (ESI⁺) [(M+H)⁺]: 266.1.

### Intermediate Int-2

### [1-(5-Chloro-1,3-benzoxazol-2-yl)pyrrolidin-3-yl]methanamine 2,2,2-trifluoroacetic acid salt

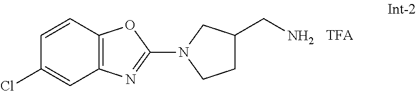 [SMILES: NCC1CCN(c2nc3cc(Cl)ccc3o2)C1]

The title compound was prepared in analogy to the procedure described for the preparation of Int-1, by using tert-butyl (pyrrolidin-3-ylmethyl)carbamate instead of tert-butyl (piperidin-4-ylmethyl)carbamate. MS obsd. (ESI⁺) [(M+H)⁺]: 252.1.

### Intermediate Int-3

### 1-(5-Chloro-1,3-benzoxazol-2-yl)piperidin-4-amine 2,2,2-trifluoroacetic acid salt

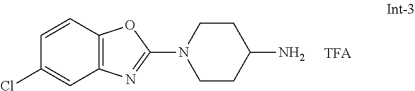 [SMILES: NC1CCN(c2nc3cc(Cl)ccc3o2)CC1]

The title compound was prepared in analogy to the procedure described for the preparation of Int-1, by using tert-butyl N-(4-piperidyl)carbamate instead of tert-butyl (piperidin-4-ylmethyl)carbamate. MS obsd. (ESI⁺) [(M+H)⁺]: 252.1.

### Intermediate Int-4

### N-[[1-(5-Chloro-1,3-benzoxazol-2-yl)-4-piperidyl]methyl]pyrrolidine-3-carboxamide 2,2,2-trifluoroacetic acid salt

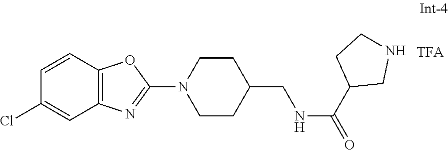 [SMILES: O=C(NCC1CCN(c2nc3cc(Cl)ccc3o2)CC1)C1CCNC1]

The title compound was prepared according to the following scheme:

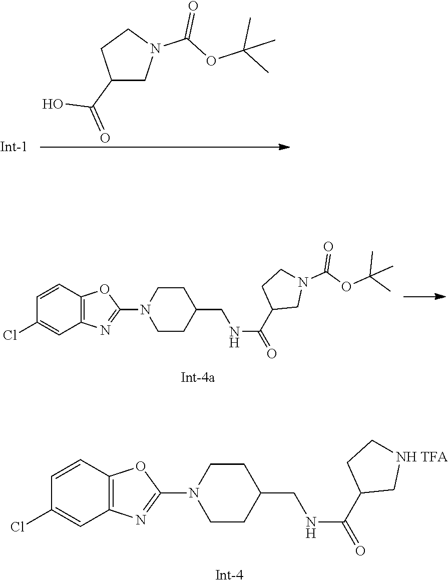

### Step 1: Preparation of tert-butyl 3-[[1-(5-chloro-1,3-benzoxazol-2-yl)-4-piperidyl]methylcarbamoyl]pyrrolidine-1-carboxylate (Int-4a)

HATU (521 mg, 1.37 mmol) was added to a stirring solution of TEA (693 mg, 954 μL, 6.85 mmol), [1-(5-chloro-1,3-benzoxazol-2-yl)-4-piperidyl]methanamine 2,2,2-trifluoroacetic acid salt (Int-1, 260 mg, 685 μmol) and 1-(tert-butoxycarbonyl)pyrrolidine-3-carboxylic acid (147 mg, 685 μmol) in CH₂Cl₂ (5 mL) at r.t. The mixture was stirred at r.t. overnight. LC-MS detection indicated that the reaction was completed. The mixture was concentrated and the residue was purified by silica gel column to afford tert-butyl 3-[[1-(5-chloro-1,3-benzoxazol-2-yl)-4-piperidyl]methylcarbamoyl]pyrrolidine-1-carboxylate (278 mg). MS obsd. (ESI⁺) [(M+H)⁺]: 463.2.

### Step 2: Preparation of [1-(5-chloro-1,3-benzoxazol-2-yl)-4-piperidyl]methanamine 2,2,2-trifluoroacetic acid salt (Int-4)

TFA (685 mg, 6 mmol) was added to a stirring solution of tert-butyl 3-(((1-(5-chlorobenzo[d]oxazol-2-yl)piperidin-4-yl)methyl)carbamoyl)pyrrolidine-1-carboxylate (Int-4a, 278 mg, 600 μmol) in CH₂Cl₂ (5 mL) at r.t. The mixture was stirred at r.t. for 2 h. LC-MS detection indicated that the reaction was completed. The mixture was concentrated to afford crude N-[[1-(5-chloro-1,3-benzoxazol-2-yl)-4-piperidyl]methyl]pyrrolidine-3-carboxamide 2,2,2-trifluoroacetic acid salt (306 mg). MS obsd. (ESI⁺) [(M+H)⁺]: 363.2.

### Intermediate Int-5

### N-[[1-(5-Chloro-1,3-benzoxazol-2-yl)-4-piperidyl]methyl]piperidine-4-carboxamide 2,2,2-trifluoroacetic acid salt

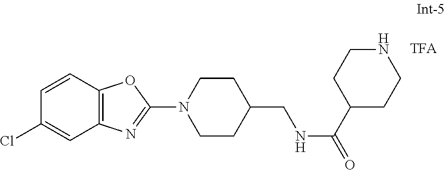 [SMILES: O=C(NCC1CCN(c2nc3cc(Cl)ccc3o2)CC1)C1CCNCC1]

The title compound was prepared in analogy to the procedure described for the preparation of Int-4, by using 1-tert-butoxycarbonylpiperidine-4-carboxylic acid instead of 1-(tert-butoxycarbonyl)pyrrolidine-3-carboxylic acid. MS obsd. (ESI⁺) [(M+H)⁺]: 377.2.

### Intermediate Int-6

### 2-(Azetidin-3-yl)-N-[[1-(5-chloro-1,3-benzoxazol-2-yl)-4-piperidyl]methyl]acetamide 2,2,2-trifluoroacetic acid salt

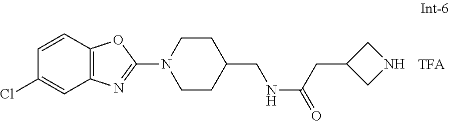 [SMILES: O=C(CC1CNC1)NCC1CCN(c2nc3cc(Cl)ccc3o2)CC1]

The title compound was prepared in analogy to the procedure described for the preparation of Int-4, by using 2-(1-tert-butoxycarbonylazetidin-3-yl)acetic acid instead of 1-(tert-butoxycarbonyl)pyrrolidine-3-carboxylic acid. MS obsd. (ESI⁺) [(M+H)⁺]: 363.2.

### Intermediate Int-7

### N-[[1-(5-Chloro-1,3-benzoxazol-2-yl)-4-piperidyl]methyl]-2-pyrrolidin-3-yl-acetamide 2,2,2-trifluoroacetic acid salt

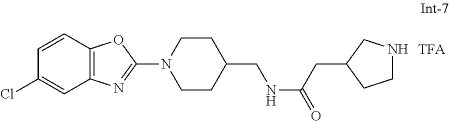 [SMILES: O=C(CC1CCNC1)NCC1CCN(c2nc3cc(Cl)ccc3o2)CC1]

The title compound was prepared in analogy to the procedure described for the preparation of Int-4, by using 2-(1-tert-butoxycarbonylpyrrolidin-3-yl)acetic acid instead of 1-(tert-butoxycarbonyl)pyrrolidine-3-carboxylic acid. MS obsd. (ESI⁺) [(M+H)⁺]: 377.2.

### Intermediate Int-8

### N-[[1-(5-Chloro-1,3-benzoxazol-2-yl)-4-piperidyl]methyl]-2-(4-piperidyl)acetamide 2,2,2-trifluoroacetic acid salt

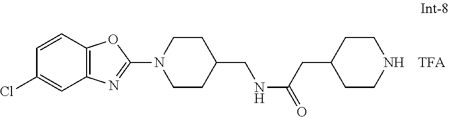 [SMILES: O=C(CC1CCNCC1)NCC1CCN(c2nc3cc(Cl)ccc3o2)CC1]

The title compound was prepared in analogy to the procedure described for the preparation of Int-4, by using 2-(1-tert-butoxycarbonyl-4-piperidyl)acetic acid instead of 1-(tert-butoxycarbonyl)pyrrolidine-3-carboxylic acid. MS obsd. (ESI⁺) [(M+H)⁺]: 391.2.

### Intermediate Int-9

### 2-(5-Chloro-1,3-benzoxazol-2-yl)-2-azaspiro[3.3]heptan-6-amine 2,2,2-trifluoroacetic acid salt

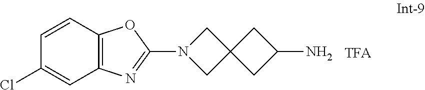 [SMILES: NC1CC2(C1)CN(c1nc3cc(Cl)ccc3o1)C2]

The title compound was prepared according to the following scheme:

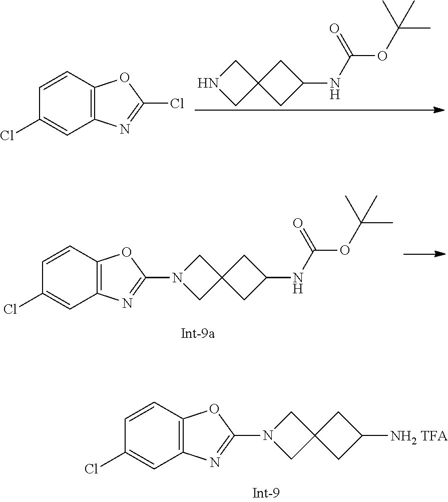

### Step 1: Preparation of tert-butyl N-[2-(5-chloro-1,3-benzoxazol-2-yl)-2-azaspiro[3.3]heptan-6-yl]carbamate (Int-9a)

2,5-Dichloro-1,3-benzoxazole (2.52 g, 13.4 mmol) was added to a solution of tert-butyl (2-azaspiro[3.3]heptan-6-yl)carbamate (2.84 g, 13.4 mmol) in CH₂Cl₂ (100 mL) and the mixture was stirred overnight at r.t. LC-MS detection indicated that the starting materials were consumed. The mixture was concentrated and the residue was purified by chromatograph on silica gel column to afford tert-butyl N-[2-(5-chloro-1,3-benzoxazol-2-yl)-2-azaspiro[3.3]heptan-6-yl]carbamate (4.47 g). MS obsd. (ESI⁺) [(M+H)⁺]:364.1.

### Step 2: Preparation of 2-(5-chloro-1,3-benzoxazol-2-yl)-2-azaspiro[3.3]heptan-6-amine 2,2,2-trifluoroacetic acid salt (Int-9)

TFA (3 g, 26.3 mmol) was added to a solution of tert-butyl N-[2-(5-chloro-1,3-benzoxazol-2-yl)-2-azaspiro[3.3]heptan-6-yl]carbamate (Int-9a, 1 g, 2.74 mmol) in CH₂Cl₂ (30 mL) and the mixture was stirred overnight at r.t. LC-MS detection indicated that the starting materials were consumed. The mixture was concentrated to afford crude 2-(5-chloro-1,3-benzoxazol-2-yl)-2-azaspiro[3.3]heptan-6-amine 2,2,2-trifluoroacetic acid salt (1.2 g). MS obsd. (ESI⁺) [(M+H)⁺]: 264.1.

### Intermediate Int-10

### 5-Chloro-2-(6-isocyanato-2-azaspiro[3.3]heptan-2-yl)-1,3-benzoxazole

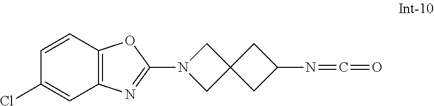 [SMILES: O=C=NC1CC2(C1)CN(c1nc3cc(Cl)ccc3o1)C2]

The title compound was prepared according to the following scheme:

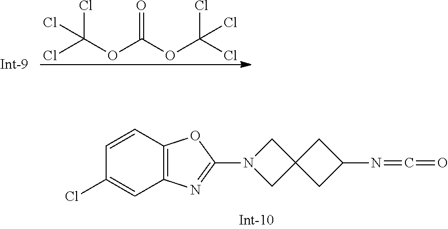 [SMILES: O=C(OC(Cl)(Cl)Cl)OC(Cl)(Cl)Cl ; O=C=NC1CC2(C1)CN(c1nc3cc(Cl)ccc3o1)C2]

Bis(trichloromethyl) carbonate (2.69 g, 9.0 mmol) was added to a solution of 2-(5-chloro-1,3-benzoxazol-2-yl)-2-azaspiro[3.3]heptan-6-amine 2,2,2-trifluoroacetic acid salt (Int-9, 1.16 g, 3.0 mmol) and NaHCO₃ (0.84 g, 10.0 mmol) in CH₂Cl₂ (50 mL) at 0° C. Then the mixture was stirred at r.t. for 3 h. The mixture was filtered and the filtrate was concentrated to afford crude 5-chloro-2-(6-isocyanato-2-azaspiro[3.3]heptan-2-yl)-1,3-benzoxazole (0.88 g). MS obsd. (ESI⁺) [(M+H)⁺]: 290.1.

### Intermediate Int-11

### 2-(6-Chloro-1,3-benzoxazol-2-yl)-2-azaspiro[3.3]heptan-6-amine 2,2,2-trifluoroacetic acid salt

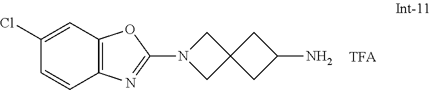 [SMILES: NC1CC2(C1)CN(c1nc3ccc(Cl)cc3o1)C2]

The title compound was prepared in analogy to the procedure described for the preparation of Int-9, by using 2,6-dichloro-1,3-benzoxazole instead of 2,5-dichloro-1,3-benzoxazole. MS obsd. (ESI⁺) [(M+H)⁺]: 264.1.

### Intermediate Int-12

### 2-(5-Chloro-1,3-benzoxazol-2-yl)-6-methyl-2-azaspiro[3.3]heptan-6-amine 2,2,2-trifluoroacetic acid salt

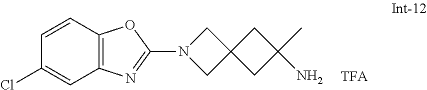 [SMILES: CC1(N)CC2(CN(c3nc4cc(Cl)ccc4o3)C2)C1]

The title compound was prepared in analogy to the procedure described for the preparation of Int-9, by using tert-butyl N-(6-methyl-2-azaspiro[3.3]heptan-6-yl)carbamate instead of tert-butyl (2-azaspiro[3.3]heptan-6-yl)carbamate. MS obsd. (ESI⁺) [(M+H)⁺]: 278.1.

### Intermediate Int-13

### 2-(5-Chloro-1,3-benzoxazol-2-yl)-2-azaspiro[3.4]octan-6-amine 2,2,2-trifluoroacetic acid salt

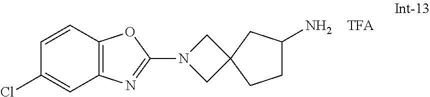 [SMILES: NC1CCC2(C1)CN(c1nc3cc(Cl)ccc3o1)C2]

The title compound was prepared in analogy to the procedure described for the preparation of Int-9, by using tert-butyl N-(2-azaspiro[3.4]octan-6-yl)carbamate instead of tert-butyl (2-azaspiro[3.3]heptan-6-yl)carbamate. MS obsd. (ESI⁺) [(M+H)⁺]: 278.1.

### Intermediate Int-14

### 2-(5-Chloro-1,3-benzoxazol-2-yl)-2-azaspiro[3.5]nonan-7-amine 2,2,2-trifluoroacetic acid slat

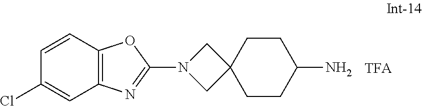 [SMILES: NC1CCC2(CC1)CN(c1nc3cc(Cl)ccc3o1)C2]

The title compound was prepared in analogy to the procedure described for the preparation of Int-9, by using tert-butyl N-(2-azaspiro[3.5]nonan-7-yl)carbamate instead of tert-butyl (2-azaspiro[3.3]heptan-6-yl)carbamate. MS obsd. (ESI⁺) [(M+H)⁺]: 292.1.

### Intermediate Int-15

### 6-(6-Chloro-1,3-benzoxazol-2-yl)-6-azaspiro[3.4]octan-2-amine 2,2,2-trifluoroacetic acid salt

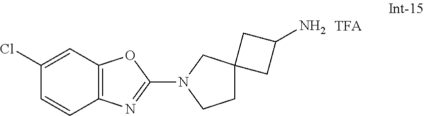 [SMILES: NC1CC2(CCN(c3nc4ccc(Cl)cc4o3)C2)C1]

The title compound was prepared in analogy to the procedure described for the preparation of Int-11, by using tert-butyl N-(6-azaspiro[3.4]octan-2-yl)carbamate instead of tert-butyl (2-azaspiro[3.3]heptan-6-yl)carbamate. MS obsd. (ESI⁺) [(M+H)⁺]: 278.1.

### Intermediate Int-16

### 6-(5-Chloro-1,3-benzoxazol-2-yl)-6-azaspiro[3.4]octan-2-amine 2,2,2-trifluoroacetic acid salt

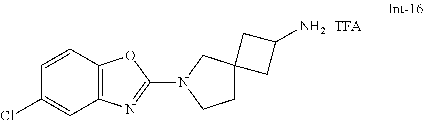 [SMILES: NC1CC2(CCN(c3nc4cc(Cl)ccc4o3)C2)C1]

The title compound was prepared in analogy to the procedure described for the preparation of Int-9, by using tert-butyl N-(6-azaspiro[3.4]octan-2-yl)carbamate instead of tert-butyl (2-azaspiro[3.3]heptan-6-yl)carbamate. MS obsd. (ESI⁺) [(M+H)⁺]: 278.1.

### Intermediate Int-17

### 2-(5-Chloro-1,3-benzoxazol-2-yl)-2-azaspiro[4.4]nonan-8-amine 2,2,2-trifluoroacetic acid salt

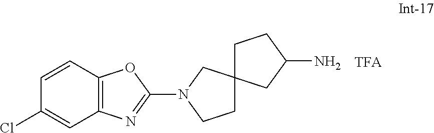 [SMILES: NC1CCC2(CCN(c3nc4cc(Cl)ccc4o3)C2)C1]

The title compound was prepared in analogy to the procedure described for the preparation of Int-9, by using tert-butyl N-(2-azaspiro[4.4]nonan-8-yl)carbamate instead of tert-butyl (2-azaspiro[3.3]heptan-6-yl)carbamate. MS obsd. (ESI⁺) [(M+H)⁺]: 292.1.

### Intermediate Int-18

### 7-(5-Chloro-1,3-benzoxazol-2-yl)-7-azaspiro[3.5]nonan-2-amine 2,2,2-trifluoroacetic acid salt

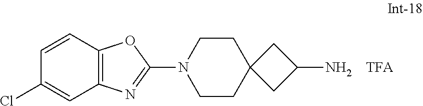 [SMILES: NC1CC2(CCN(c3nc4cc(Cl)ccc4o3)CC2)C1]

The title compound was prepared in analogy to the procedure described for the preparation of Int-9, by using tert-butyl N-(7-azaspiro[3.5]nonan-2-yl)carbamate instead of tert-butyl (2-azaspiro[3.3]heptan-6-yl)carbamate. MS obsd. (ESI⁺) [(M+H)⁺]: 292.1.

### Intermediate Int-19

### 3-(5-Chloro-1,3-benzoxazol-2-yl)-3-azaspiro[5.5]undecan-9-amine 2,2,2-trifluoroacetic acid salt

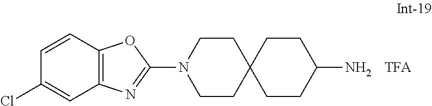 [SMILES: NC1CCC2(CC1)CCN(c1nc3cc(Cl)ccc3o1)CC2]

The title compound was prepared in analogy to the procedure described for the preparation of Int-9, by using tert-butyl N-(3-azaspiro[5.5]undecan-9-yl)carbamate instead of tert-butyl (2-azaspiro[3.3]heptan-6-yl)carbamate. MS obsd. (ESI⁺) [(M+H)⁺]: 320.1.

### Intermediate Int-20

### 3-(6-Chloro-1,3-benzoxazol-2-yl)-3-azaspiro[5.5]undecan-9-amine 2,2,2-trifluoroacetic acid salt

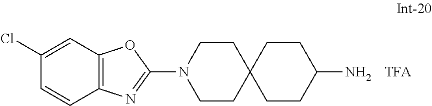 [SMILES: NC1CCC2(CC1)CCN(c1nc3ccc(Cl)cc3o1)CC2]

The title compound was prepared in analogy to the procedure described for the preparation of Int-11, by using tert-butyl N-(3-azaspiro[5.5]undecan-9-yl)carbamate instead of tert-butyl (2-azaspiro[3.3]heptan-6-yl)carbamate. MS obsd. (ESI⁺) [(M+H)⁺]: 320.1.

### Intermediate Int-21

### 3-(1,3-Benzoxazol-2-yl)-3-azaspiro[5.5]undecan-9-amine 2,2,2-trifluoroacetic acid salt

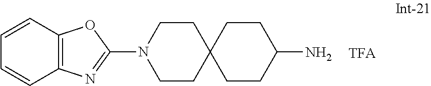 [SMILES: NC1CCC2(CC1)CCN(c1nc3ccccc3o1)CC2]

The title compound was prepared in analogy to the procedure described for the preparation of Int-9, by using tert-butyl N-(3-azaspiro[5.5]undecan-9-yl)carbamate instead of tert-butyl (2-azaspiro[3.3]heptan-6-yl)carbamate, 2-chloro-1,3-benzoxazole instead of 2,5-dichloro-1,3-benzoxazole. MS obsd. (ESI⁺) [(M+H)⁺]: 286.2.

### Intermediate Int-22

### 2-Carbamoylpyridine-4-carboxylic acid

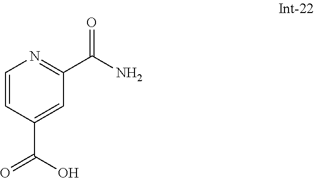 [SMILES: NC(=O)c1cc(C(=O)O)ccn1]

The title compound was prepared according to the following scheme:

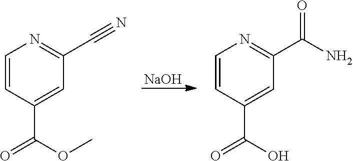

NaOH (1.00 g, 25.0 mmol) was added to the solution of methyl 2-cyanopyridine-4-carboxylate (810 mg, 5.0 mmol) in methanol (20 mL) and water (10 mL) at r.t. Then the mixture was refluxed for 2 h. LC-MS detection indicated that the reaction was completed. The organic phase was removed and the mixture was acidified to pH=2. The mixture was filtered and dried to afford 2-carbamoylpyridine-4-carboxylic acid (530 mg). MS obsd. (ESI⁺) [(M+H)⁺]: 167.1.

### Intermediate Int-23

### 5-Methylsulfinylfuran-2-carboxylic acid

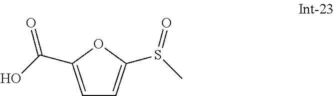

The title compound was prepared according to the following scheme:

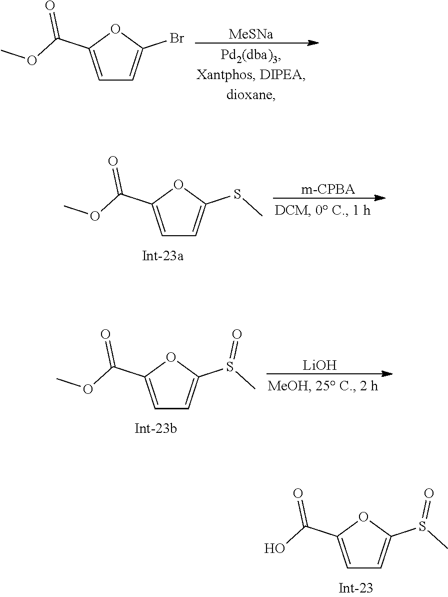

### Step 1: Preparation of methyl 5-methylsulfanylfuran-2-carboxylate (Int-23a)

A mixture of methyl 5-bromo-2-furoate (10 g, 48.78 mmol), sodium thiomethoxide (6.84 g, 97.56 mmol), 9,9-dimethyl-4,5-bis(diphenylphosphino)xanthene (2.82 g, 4.88 mmol), N,N-diisopropylethylamine (25.49 mL, 146.33 mmol) and tris(dibenzylideneacetone)dipalladium (0) (2.23 g, 2.44 mmol) in 1,4-dioxane (200 mL) was stirred at 110° C. for 15 h. The solvent was evaporated in vacuo and the EtOAc (300 mL) was added. The resulting mixture was filtered on a short silica gel column. The filtrate was concentrated and purified by flash column (eluting with EtOAc/PE=3/97) to give methyl 5-methylsulfanylfuran-2-carboxylate as a yellow oil (7.0 g). MS obsd. (ESI⁺) [(M+H)⁺]: 173.1.

### Step 2: Preparation of methyl 5-methylsulfinylfuran-2-carboxylate (Int-23b)

To a solution of methyl 5-methylsulfanylfuran-2-carboxylate (4.5 g, 26.1 mmol) in CH₂Cl₂ (10 mL) was added m-CPBA (4.5 g, 26.1 mmol). After being stirred at 0° C. for 1 h, the mixture was washed with saturated Na₂CO₃. The organic layer was dried over Na₂SO₄, filtered and concentrated in vacuo. The residue was purified by flash column (eluting with EA/PE=1/2) to give methyl 5-methylsulfinylfuran-2-carboxylate as a light yellow solid (3.7 g). MS obsd. (ESI⁺) [(M+H)⁺]: 189.1.

### Step 3: Preparation of 5-methylsulfinylfuran-2-carboxylic acid (Int-23)

LiOH·H₂O (134 mg, 2.4 mmol) was added to a solution of methyl 5-methylsulfinylfuran-2-carboxylate (90 mg, 0.48 mmol) in a mixed solvent of MeOH (10 mL) and water (10 mL). After being stirred at 25° C. for 2 h, the MeOH was evaporated. The residue was acidified by HCl (1 M) to pH=2 and extracted with CH₂Cl₂ (15 mL×3). The combined organic layer was dried over Na₂SO₄, filtered and concentrated in vacuo to give 5-methylsulfinylfuran-2-carboxylic acid as a yellow oil (60 mg). MS obsd. (ESI⁺) [(M+H)⁺]: 175.1.

### Intermediate Int-24

### 5-Methylsulfonylfuran-2-carboxylic acid

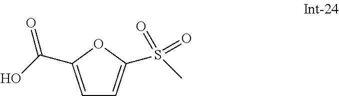

The title compound was prepared according to the following scheme:

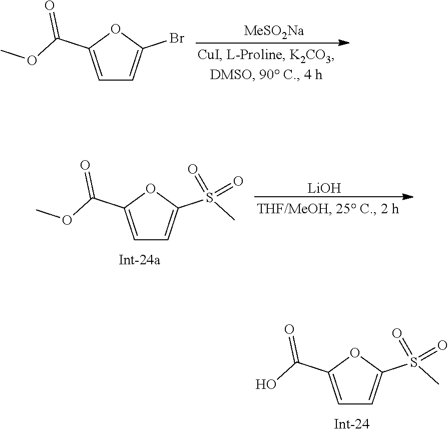

### Step 1: Preparation of methyl 5-methylsulfonylfuran-2-carboxylate (Int-24a)

To a solution of methyl 5-bromo-2-furoate (2.05 g, 10 mmol) in DMSO (25 mL) was added methylsulfinyloxysodium (1.23 g, 12 mmol) followed by copper (I) iodide (380.9 mg, 2 mmol), L-proline (460.5 mg, 4 mmol) and K₂CO₃ (414.6 mg, 3 mmol). The resulting mixture was heated with stirring at 90° C. for 4 h. The reaction was diluted with water (30 mL) and extracted with EtOAc (30 mL×3). The combined organic layer was washed with brine (50 mL×2), dried over Na₂SO₄, filtered and concentrated in vacuo. The residue was purified by flash column (eluting with EtOAc/PE=1/3) to give methyl 5-methylsulfonylfuran-2-carboxylate as a white solid (750 mg). MS obsd. (ESI⁺) [(M+H)⁺]: 205.1.

### Step 2: Preparation of 5-methylsulfonylfuran-2-carboxylic acid (Int-24)

An aqueous of LiOH·H₂O (2 M, 5.5 mL) was added to a solution of methyl 5-methylsulfonylfuran-2-carboxylate (750 mg, 3.7 mmol) in a mixed solvent of THF (25 mL) and MeOH (25 mL). After being stirred at 25° C. for 2 h, most of the solvent was evaporated. The residue was acidified by HCl (2 M) to pH=3 and extracted with EtOAc (20 mL×3). The combined organic layer was dried over Na₂SO₄, filtered and concentrated in vacuo to give 5-methylsulfonylfuran-2-carboxylic acid as a white solid (655 mg). MS obsd. (ESI⁺) [(M+H)⁺]: 191.1.

### Intermediate Int-25

### 5-Ethylsulfinylfuran-2-carboxylic acid

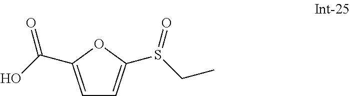

The title compound was prepared according to the following scheme:

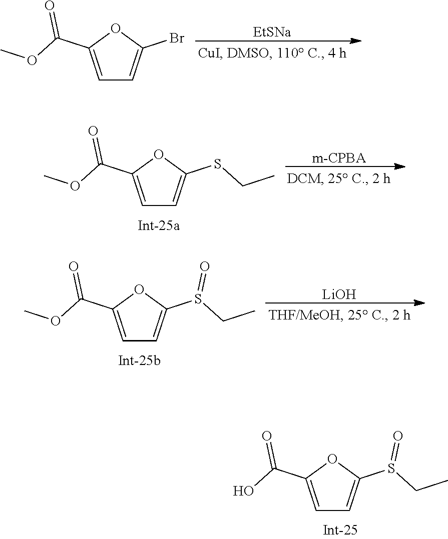

### Step 1: Preparation of methyl 5-ethylsulfanylfuran-2-carboxylate (Int-25a)

To a solution of methyl 5-bromo-2-furoate (610 mg, 3 mmol) in DMSO (6 mL) was added ethylsulfanylsodium (501 mg, 6 mmol) followed by copper (I) iodide (567 mg, 3 mmol). The mixture was then heated with stirring at 110° C. for 4 h. The reaction was diluted with water (30 mL) and extracted with EtOAc (20 mL×3). The combined organic layer was washed with brine (50 mL×2), dried over Na₂SO₄, filtered and concentrated to afford methyl 5-ethylsulfanylfuran-2-carboxylate as a brown oil (318 mg), which was used for the next step without further purification. MS obsd. (ESI⁺) [(M+H)⁺]: 187.1.

### Step 2: Preparation of methyl 5-ethylsulfinylfuran-2-carboxylate (Int-25b)

To a solution of methyl 5-ethylsulfanylfuran-2-carboxylate (374 mg, 2.0 mmol) in CH₂Cl₂ (10 mL) was added m-CPBA (345 mg, 2 mmol). After being stirred at 0° C. for 1 h, the mixture was washed with saturated Na₂CO₃. The organic layer was dried over Na₂SO₄, filtered and concentrated in vacuo. The residue was purified by flash column (eluting with EtOAc/PE=1/3) to give methyl 5-ethylsulfinylfuran-2-carboxylate as a light yellow oil (312 mg). MS obsd. (ESI⁺) [(M+H)⁺]: 203.1.

### Step 3: Preparation of 5-ethylsulfinylfuran-2-carboxylic acid (Int-25)

An aqueous of LiOH·H₂O (2 M, 5.5 mL) was added to a solution of methyl 5-ethylsulfonylfuran-2-carboxylate (244 mg, 1.2 mmol) in a mixed solvent of THF (5 mL) and MeOH (5 mL). After being stirred at 25° C. for 2 h, most of the solvent was evaporated. The residue was acidified by HCl (2 M) to pH=3 and extracted with EtOAc (5 mL×3). The combined organic layer was dried over Na₂SO₄, filtered and concentrated in vacuo to give 5-ethylsulfinylfuran-2-carboxylic acid as a light yellow solid (220 mg), which was used for the next step without further purification. MS obsd. (ESI⁺) [(M+H)⁺]: 189.1.

### Intermediate Int-26

### 5-Ethylsulfonylfuran-2-carboxylic acid

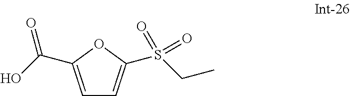

The title compound was prepared according to the following scheme:

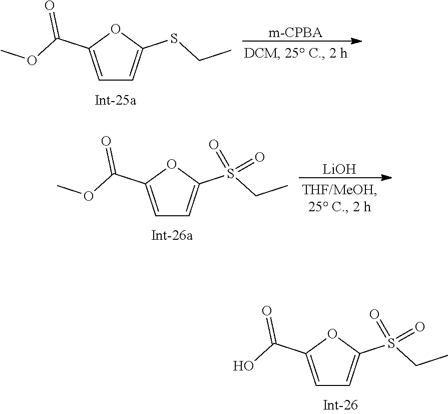

### Step 1: Preparation of methyl 5-ethylsulfonylfuran-2-carboxylate (Int-26a)

To a solution of methyl 5-ethylsulfanylfuran-2-carboxylate (310 mg, 1.7 mmol) in CH₂Cl₂ (10 mL) was added m-CPBA (862 mg, 5 mmol). After being stirred at 25° C. for 2 h, the mixture was washed with saturated NaHCO₃. The organic layer was dried over Na₂SO₄, filtered and concentrated in vacuo. The residue was purified by flash column (eluting with EtOAc/PE=1/3) to give methyl 5-ethylsulfonylfuran-2-carboxylate as a light yellow oil (240 mg). MS obsd. (ESI⁺) [(M+H)⁺]: 219.1.

### Step 2: Preparation of 5-ethylsulfonylfuran-2-carboxylic acid (Int-26)

An aqueous of LiOH·H₂O (2 M, 5.5 mL) was added to a solution of methyl 5-ethylsulfonylfuran-2-carboxylate (240 mg, 1.1 mmol) in a mixed solvent of THF (5 mL) and MeOH (5 mL). After being stirred at 25° C. for 2 h, most of the solvent was evaporated. The residue was acidified by HCl (2 M) to pH=3 and extracted with EtOAc (5 mL×3). The combined organic layer was dried over Na₂SO₄, filtered and concentrated in vacuo to give 5-ethylsulfonylfuran-2-carboxylic acid as a light yellow solid (220 mg), which was used for the next step without further purification. MS obsd. (ESI⁺) [(M+H)⁺]: 205.1.

### Intermediate Int-27

### 5-Cyclopropylsulfonylfuran-2-carboxylic acid

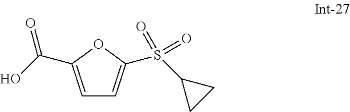 [SMILES: O=C(O)c1ccc(S(=O)(=O)C2CC2)o1]

The title compound was prepared in analogy to the procedure described for the preparation of 5-methylsulfonylfuran-2-carboxylic acid Int-24, by using cyclopropylsulfinyloxysodium instead of methylsulfinyloxysodium. MS obsd. (ESI⁺) [(M+H)⁺]: 217.1.

### Intermediate Int-28

### 5-(Cyclopropylmethylsulfinyl)furan-2-carboxylic acid

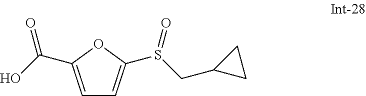 [SMILES: O=C(O)c1ccc(S(=O)CC2CC2)o1]

The title compound was prepared according to the following scheme:

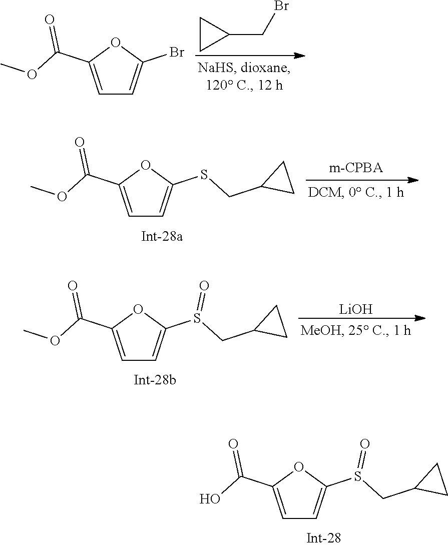

### Step 1: Preparation of methyl 5-(cyclopropylmethylsulfanyl)furan-2-carboxylate (Int-28a)

To a solution of methyl 5-bromo-2-furoate (2 g, 9.76 mmol) in 1,4-dioxane (20 mL) was added sodium hydrosulfide (5.5 g, 97.6 mmol) and (bromomethyl)cyclopropane (3.32 mL, 34.1 mmol). After being stirred at 120° C. for 12 h, the reaction mixture was quenched with H₂O (50 mL) and extracted with CH₂Cl₂ (50 mL×3). The combined organic layer was washed with brine, dried over Na₂SO₄ and concentrated in vacuo. The residue was purified by flash column (eluting with 100% PE) to afford methyl 5-(cyclopropylmethylsulfanyl)furan-2-carboxylate as a colorless oil (580 mg, 28%). MS obsd. (ESI⁺) [(M+H)⁺]: 213.1. ¹H NMR (400 MHz, CDCl₃) δ ppm: 7.14 (d, J=3.5 Hz, 1H), 6.50 (d, J=3.5 Hz, 1H), 3.89 (s, 3H), 2.86 (d, J=7.2 Hz, 2H), 0.95-1.10 (m, 1H), 0.51-0.61 (m, 2H), 0.14-0.24 (m, 2H).

### Step 2: Preparation of methyl 5-(cyclopropylmethylsulfinyl)furan-2-carboxylate (Int-28b)

To a solution of 5-(cyclopropylmethylsulfanyl)furan-2-carboxylate (382 mg, 1.8 mmol) in CH₂Cl₂ (10 mL) was added m-CPBA (310 mg, 1.8 mmol). After being stirred at 0° C. for 1 h, the mixture was washed with saturated NaHCO₃. The organic layer was dried over Na₂SO₄, filtered and concentrated in vacuo. The residue was purified by flash column (eluting with EtOAc/PE=1/2) to give methyl 5-(cyclopropylmethylsulfinyl)furan-2-carboxylate as a light yellow oil (380 mg, 92.5%). MS obsd. (ESI⁺) [(M+H)⁺]: 229.1.

### Step 3: Preparation of 5-(cyclopropylmethylsulfinyl)furan-2-carboxylic acid (Int-28)

To a solution of methyl 5-(cyclopropylmethylsulfinyl)furan-2-carboxylate (380 mg, 1.66 mmol) in a mixed solvent of MeOH (5 mL) was added LiOH·H₂O (2.5 mL, 2 M). After being stirred at 25° C. for 1 h, most of the solvent was evaporated. The residue was acidified by 3 mL of HCl (2 M) and extracted with EtOAc (20 mL). The organic layer was dried over Na₂SO₄, filtered and concentrated in vacuo to give 5-(cyclopropylmethylsulfinyl)furan-2-carboxylic acid as a white solid (320 mg, 89.7%). MS obsd. (ESI⁺) [(M+H)⁺]: 215.1.

### Intermediate Int-29

### 5-(Cyclopropylmethylsulfonyl)furan-2-carboxylic acid

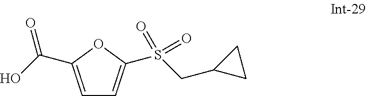 [SMILES: O=C(O)c1ccc(S(=O)(=O)CC2CC2)o1]

The title compound was prepared according to the following scheme:

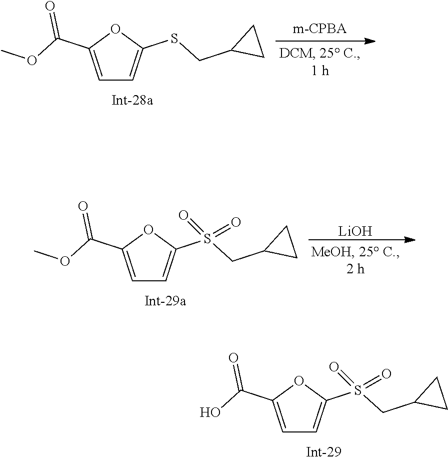

### Step 1: Preparation of methyl 5-(cyclopropylmethylsulfonyl)furan-2-carboxylate (Int-29a)

To a solution of methyl 5-(cyclopropylmethylsulfanyl)furan-2-carboxylate (272 mg, 0.69 mmol) in CH₂Cl₂ (10 mL) was added m-CPBA (358 mg, 2 mmol). After being stirred at 25° C. for 1 h, the mixture was washed with saturated NaHCO₃. The organic layer was dried over Na₂SO₄, filtered and concentrated in vacuo. The residue was purified by flash column (eluting with EtOAc/PE=0 to 20%) to give methyl 5-(cyclopropylmethylsulfonyl)furan-2-carboxylate as a colorless oil (110 mg, 59.8%). MS obsd. (ESI⁺) [(M+H)⁺]: 245.1. ¹H NMR (400 MHz, CDCl₃) δ ppm: 7.22-7.25 (m, 2H), 3.94 (d, J=4.2 Hz, 3H), 3.21 (d, J=7.3 Hz, 2H), 1.02-1.18 (m, 1H), 0.56-0.68 (m, 2H), 0.14-0.26 (m, 2H).

### Step 2: Preparation of 5-(cyclopropylmethylsulfonyl)furan-2-carboxylic acid (Int-29)

To a solution of methyl 5-(cyclopropylmethylsulfonyl)furan-2-carboxylate (101 mg, 0.41 mmol) in a mixed solvent of MeOH (9 mL) and water (3 mL) was added LiOH (50 mg, 2 mmol). After being stirred at 25° C. for 2 h, most of the solvent was evaporated. The residue was acidified by HCl (1 M) to pH=2 and extracted with CH₂Cl₂ (30 mL×3). The combined organic layer was dried over Na₂SO₄, filtered and concentrated in vacuo to give 5-(cyclopropylmethylsulfonyl)furan-2-carboxylic acid as an off-white solid (85 mg, 84.8%). MS obsd. (ESI⁺) [(M+H)⁺]: 231.0.

### Intermediate Int-30

### 5-Isobutylsulfinylfuran-2-carboxylic acid

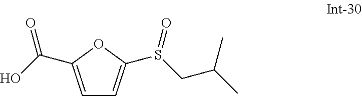

The title compound was prepared in analogy to the procedure described for the preparation of Int-28, by using 1-bromo-2-methylpropane instead of (bromomethyl)cyclopropane. MS obsd. (ESI⁺) [(M+H)⁺]: 217.1.

### Intermediate Int-31

### 5-Isobutylsulfonylfuran-2-carboxylic acid

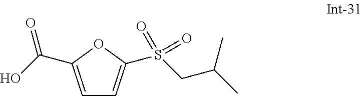

The title compound was prepared in analogy to the procedure described for the preparation of Int-29, by using 1-bromo-2-methylpropane instead of (bromomethyl)cyclopropane. MS obsd. (ESI⁺) [(M+H)⁺]: 233.1.

### Intermediate Int-32

### 5-(Methylsulfonylmethyl)furan-2-carboxylic acid

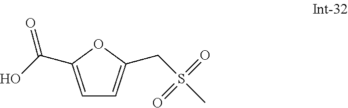

The title compound was prepared according to the following scheme:

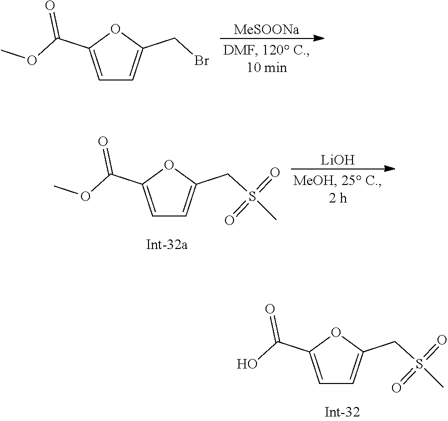

### Step 1: Preparation of methyl 5-(methylsulfonylmethyl)furan-2-carboxylate (Int-32a)

Sodium methanesulfinate (59.2 mg, 0.58 mmol) was added to a solution of 5-(bromomethyl)furan-2-carboxylate (116 mg, 0.53 mmol) in DMF (5 mL) at r.t. Then the mixture was stirred at 120° C. under MW for 10 min. The solvent was removed and the residue was purified by flash column to afford methyl 5-(methylsulfonylmethyl)furan-2-carboxylate (108 mg). MS obsd. (ESI⁺) [(M+H)⁺]: 219.0.

### Step 2: Preparation of 5-(methylsulfonylmethyl)furan-2-carboxylic acid (Int-32)

To a solution of methyl methyl 5-(methylsulfonylmethyl)furan-2-carboxylate (100 mg, mmol) in a mixed solvent of MeOH (5 mL) and water (5 mL) was added LiOH (609 mg, mmol) and the reaction was stirred at 25° C. for 2 h. The resulting mixture was concentrated to remove the MeOH and the residue was acidified to pH=2 with 1 M HCl, extracted with CH₂Cl₂ (40 mL×3). The combined organic layer was dried by anhydrous Na₂SO₄ and concentrated under vacuo to give 5-(methylsulfonylmethyl)furan-2-carboxylic acid (90 m). MS obsd. (ESI⁺) [(M+H)⁺]: 205.0.

### Intermediate Int-33

### 5-(Methylsulfonimidoyl)furan-2-carboxylic acid

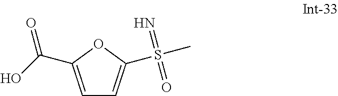

The title compound was prepared according to the following scheme:

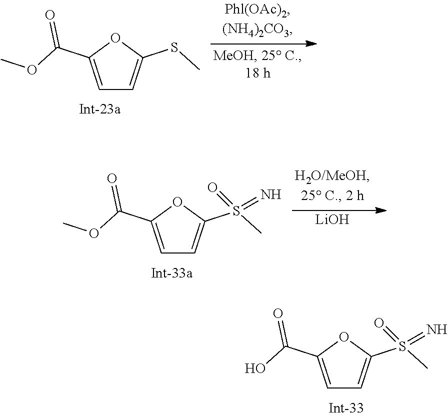

### Step 1: Preparation of methyl 5-(methylsulfonimidoyl)furan-2-carboxylate (Int-33a)

A mixture of iodobenzene diacetate (1.72 g, 9.24 mmol) and ammonium carbonate (289.6 mg, 3.01 mmol) in methanol (50 mL) was stirred at 25° C. for 5 min. And then methyl 5-methylsulfanylfuran-2-carboxylate (0.69 g, 4.00 mmol) was added. After being stirred at 25° C. for 18 h, the mixture was quenched by water, concentrated in vacuo to remove the organic solvent. The residue was extracted with CH₂Cl₂ (50 mL×3). The combined organic layers were washed with brine (50 mL×3), dried over Na₂SO₄, filtered and concentrated under reduced pressure. The residue was purified by chromatography on silica gel column to give methyl 5-(methylsulfonimidoyl)furan-2-carboxylate (633 mg, 78%). MS obsd. (ESI⁺) [(M+H)⁺]: 204.1.

### Step 2: Preparation of 5-(methylsulfonimidoyl)furan-2-carboxylic acid (Int-33)

To a solution of methyl 5-(methylsulfonimidoyl)furan-2-carboxylate (90 mg, 0.44 mmol) in a mixed solvent of MeOH (10 mL) and water (10 mL) was added LiOH·H₂O (134 mg, 2.4 mmol). After being stirred at 25° C. for 2 h, the MeOH was evaporated. The residue was acidified by HCl (1 M) to pH=3 and extracted with CH₂Cl₂ (15 mL×3). The combined organic layer was dried over Na₂SO₄, filtered and concentrated in vacuo to give 5-(methylsulfonimidoyl)furan-2-carboxylic acid (56 mg). MS obsd. (ESI⁺) [(M+H)⁺]: 190.1.

### Intermediate Int-34

### 5-(Ethylsulfonimidoyl)furan-2-carboxylic acid

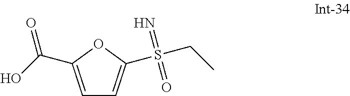

The title compound was prepared in analogy to the procedure described for the preparation of Int-33, by using methyl 5-ethylsulfanylfuran-2-carboxylate instead of methyl 5-methylsulfanylfuran-2-carboxylate. MS obsd. (ESI⁺) [(M+H)⁺]: 204.1.

### Intermediate Int-35

### 5-(Cyclopropylmethylsulfonimidoyl)furan-2-carboxylic acid

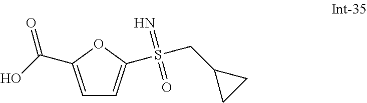 [SMILES: N=S(=O)(CC1CC1)c1ccc(C(=O)O)o1]

The title compound was prepared in analogy to the procedure described for the preparation of Int-33, by using methyl 5-(cyclopropylmethylsulfanyl)furan-2-carboxylate instead of methyl 5-methylsulfanylfuran-2-carboxylate. MS obsd. (ESI⁺) [(M+H)⁺]: 230.1.

### Intermediate Int-36

### 5-(Isobutylsulfonimidoyl)furan-2-carboxylic acid

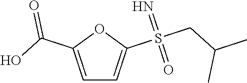

### Int-36

The title compound was prepared in analogy to the procedure described for the preparation of Int-33, by using methyl 5-isobutylsulfanylfuran-2-carboxylate instead of methyl 5-methylsulfanylfuran-2-carboxylate. MS obsd. (ESI⁺) [(M+H)⁺]: 232.1.

### Intermediate Int-37

### 5-(Cyclopropylsulfamoyl)furan-2-carboxylic acid

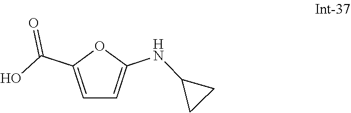 [SMILES: O=C(O)c1ccc(NC2CC2)o1]

The title compound was prepared according to the following scheme:

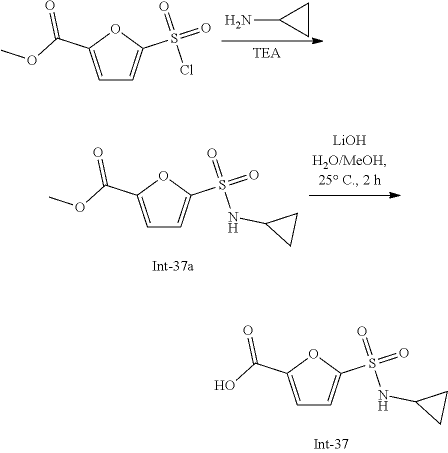

### Step 1: Preparation of methyl 5-(cyclopropylsulfamoyl)furan-2-carboxylate (Int-37a)

Methyl 5-(chlorosulfonyl)furan-2-carboxylate (500 mg, 2.23 mmol) was added to a solution of cyclopropanamine (127 mg, 2.23 mmol) and TEA (676 mg, 931 μl, 6.68 mmol) in CH₂Cl₂ (10 mL) and stirred at r.t. for 3 h. LC-MS detection indicated that the reaction was completed. Then the reaction mixture was concentrated to afford the crude methyl 5-(cyclopropylsulfamoyl)furan-2-carboxylate. MS obsd. (ESI⁺) [(M+H)⁺]: 246.0.

### Step 2: Preparation of 5-(cyclopropylsulfamoyl)furan-2-carboxylic acid (Int-37)

LiOH·H₂O (134 mg, 2.4 mmol) was added to a solution of methyl 5-(cyclopropylsulfamoyl)furan-2-carboxylate (100 mg, 0.41 mmol) in a mixed solvent of MeOH (10 mL) and water (10 mL). After being stirred at 25° C. for 2 h, the MeOH was evaporated. The residue was acidified by HCl (1 M) to pH=3 and extracted with CH₂Cl₂ (15 mL×3). The combined organic layer was dried over Na₂SO₄, filtered and concentrated in vacuo to give 5-(cyclopropylsulfamoyl)furan-2-carboxylic acid (73 mg). MS obsd. (ESI⁺) [(M+H)⁺]: 232.0.

### Intermediate Int-38

### 5-[(3,3-Difluorocyclobutyl)sulfamoyl]furan-2-carboxylic acid

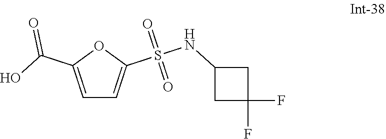 [SMILES: O=C(O)c1ccc(S(=O)(=O)NC2CC(F)(F)C2)o1]

The title compound was prepared in analogy to the procedure described for the preparation of Int-37, by using 3,3-difluorocyclobutanamine instead of cyclopropanamine. MS obsd. (ESI⁺) [(M+H)⁺]: 282.2.

### Intermediate Int-39

### 2-(2-Methoxyethoxy)pyridine-4-carboxylic acid

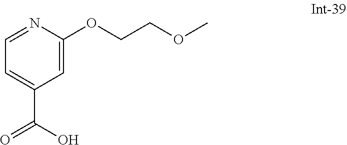

The title compound was prepared according to the following scheme:

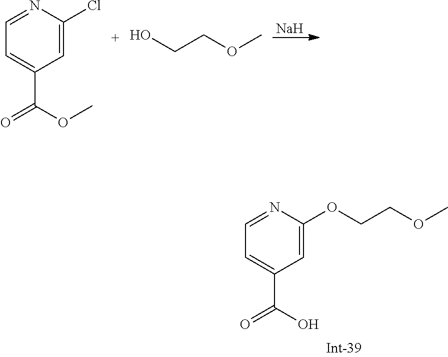

Sodium hydride (209.81 mg, 8.74 mmol) was added to a solution of 2-methoxyethanol (665.2 mg, 8.74 mmol) in DMF (5 mL) at 0° C. and the mixture was stirred at 0° C. for 30 min. Then methyl 2-chloroisonicotinate (300.0 mg, 1.75 mmol) was added and the mixture was stirred for 16 h at 90° C. LC-MS detection indicated that starting materials was consumed completely. The mixture was diluted with water (20 mL) and adjusted pH to 4 by 1 N HCl aqueous solution, extracted with CH₂Cl₂ (15 mL×3). The combined organics was dried and concentrated. The residue was purified by prep-HPLC to afford 2-(2-methoxyethoxy)pyridine-4-carboxylic acid (110 mg). MS obsd. (ESI⁺) [(M+H)⁺]: 198.1.

### Intermediate Int-40

### 2-Isopropoxypyridine-4-carboxylic acid

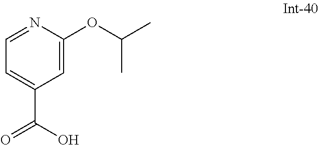

The title compound was prepared according to the following scheme:

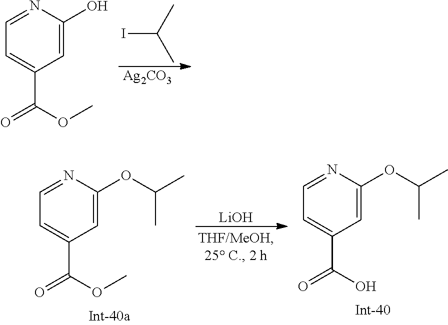

### Step 1: Preparation of methyl 2-isopropoxypyridine-4-carboxylate (Int-40a)

2-Iodopropane (6.66 g, 39.2 mmol) and silver carbonate (4.32 g, 15.7 mmol) was added to a solution of methyl 2-hydroxypyridine-4-carboxylate (1.20 g, 7.8 mmol) in CHCl₃ (20 mL) at 25° C. and the mixture was stirred at 25° C. for 3 h. LC-MS detection indicated that starting materials were consumed completely. The mixture was diluted with water (20 mL) and extracted with CH₂Cl₂ (15 mL×3). The combined organics was dried and concentrated. The residue was purified by flash column chromatography to afford methyl 2-isopropoxypyridine-4-carboxylate (1.30 g). MS obsd. (ESI⁺) [(M+H)⁺]: 196.1.

### Step 2: Preparation of 2-isopropoxypyridine-4-carboxylic acid (Int-40)

An aqueous of LiOH·H₂O (2 M, 16 mL) was added to a solution of methyl 2-isopropoxypyridine-4-carboxylate (1.30 g, 6.7 mmol) in a mixed solvent of H₂O (8 mL), THF (10 mL) and MeOH (10 mL). After being stirred at 25° C. for 2 h, most of the solvent was evaporated. The residue was acidified by HCl (2 M) to pH=3 and extracted with EtOAc (5 mL×3). The combined organic layer was dried over Na₂SO₄, filtered and concentrated in vacuo to give 2-isopropoxypyridine-4-carboxylic acid (1.1 g), which was used for the next step without further purification. MS obsd. (ESI⁺) [(M+H)⁺]: 182.1.

### Intermediate Int-41

### 2-Methylsulfonylpyridine-4-carboxylic acid

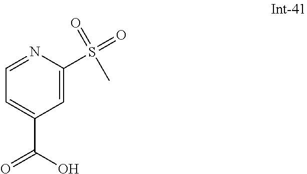

The title compound was prepared according to the following scheme:

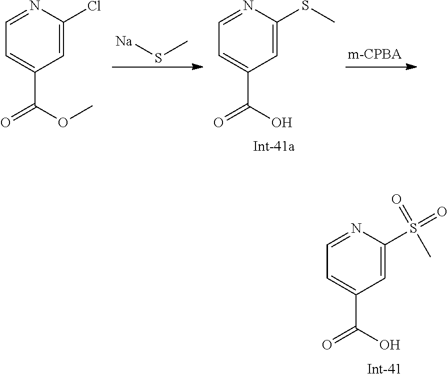

### Step 1: Preparation of 2-methylsulfanylpyridine-4-carboxylic acid (Int-41a)

To a solution of methyl 2-chloroisonicotinate (1.0 g, 5.83 mmol) in DMF (20 mL) was added sodium thiomethoxide (3.27 g, 23.31 mmol). The mixture was stirred for 2 h at 25° C. LC-MS detection indicated that starting materials were consumed completely. The mixture was diluted with water (200 mL), acidified by HCl (2 M) to pH=3 and extracted with CH₂Cl₂ (150 mL×3). The combined organics was dried and concentrated. The residue was purified by prep-HPLC to afford 2-methylsulfanylpyridine-4-carboxylic acid (560 mg). MS obsd. (ESI⁺) [(M+H)⁺]: 170.0.

### Step 2: Preparation of 2-methylsulfonylpyridine-4-carboxylic acid (Int-41)

To a solution of 2-methylsulfanylpyridine-4-carboxylic acid (500 mg, 2.96 mmol) in CH₂Cl₂ (50 mL) was added m-CPBA (1274.9 mg, 7.39 mmol). The mixture was stirred for 2 h at 25° C. Then LC-MS detection showed the reaction was completed. The mixture was concentrated to dryness and purified by Prep-HPLC to afford 2-methylsulfonylpyridine-4-carboxylic acid (155 mg). MS obsd. (ESI⁺) [(M+H)⁺]: 202.1.

### Intermediate Int-42

### 2-Ethylsulfonylpyridine-4-carboxylic acid

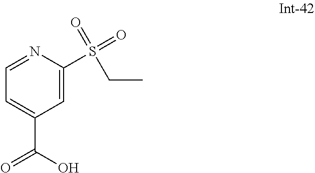

The title compound was prepared according to the following scheme:

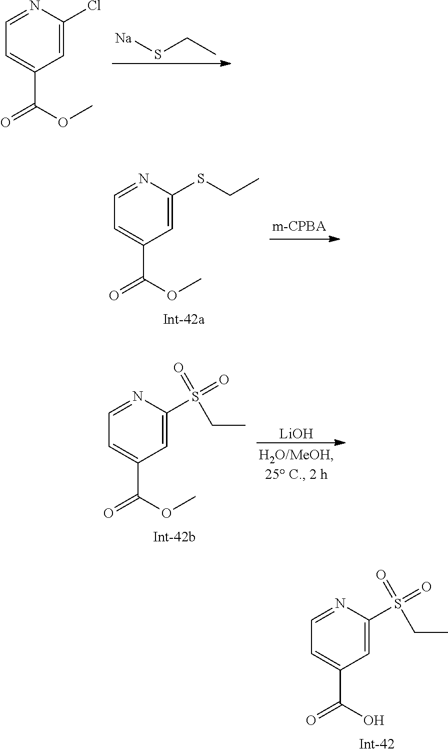

### Step 1: Preparation of methyl 2-ethylsulfanylpyridine-4-carboxylate (Int-42a)

To a solution of methyl 2-chloroisonicotinate (1.3 g, 7.58 mmol) in DMF (12 mL) was added sodium ethanethiolate (764 mg, 9.09 mmol). The mixture was stirred for 12 h at 25° C. LC-MS detection indicated that starting materials were consumed completely. The mixture was concentrated to dryness in vacuo to remove DMF. Then the residue was purified by flash column chromatography to give methyl 2-ethylsulfanylpyridine-4-carboxylate (796 mg). MS obsd. (ESI⁺) [(M+H)⁺]: 198.1.

### Step 2: Preparation of methyl 2-ethylsulfonylpyridine-4-carboxylate (Int-42b)

To a solution of methyl 2-ethylsulfanylpyridine-4-carboxylate (523.0 mg, 2.65 mmol) in CH₂Cl₂ (10 mL) was added m-CPBA (1372.7 mg, 7.95 mmol). The mixture was stirred for 1 h at 25° C. Then LC-MS detection showed the reaction was completed. The mixture was washed by NaHCO₃ aqueous solution and the organic layer was concentrated to dryness. The residue was purified by flash column chromatography to afford methyl 2-ethylsulfonylpyridine-4-carboxylate (600 mg). MS obsd. (ESI⁺) [(M+H)⁺]: 230.1.

### Step 3: Preparation of 2-ethylsulfonylpyridine-4-carboxylic acid (Int-42)

To a solution of methyl 2-ethylsulfanylpyridine-4-carboxylate (600 mg, 3.0 mmol) in methanol (10 mL) and water (10 mL) was added lithium hydroxide monohydrate (630 mg, 15.0 mmol) at 25° C., then the mixture was stirred for 2 h. LC-MS detection showed the reaction was completed. The mixture was concentrated to remove the MeOH, acidified to pH=2 with 1 N HCl and extracted with CH₂Cl₂ (15 mL×3). The organic layer was dried and concentrated. The residue was used for the next step without further purification (610 mg). MS obsd. (ESI⁺) [(M+H)⁺]: 216.1.

### Intermediate Int-43

### 2-Cyclopropylsulfonylpyridine-4-carboxylic acid

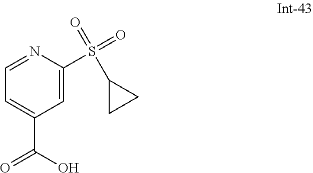 [SMILES: O=C(O)c1ccnc(S(=O)(=O)C2CC2)c1]

The title compound was prepared according to the following scheme:

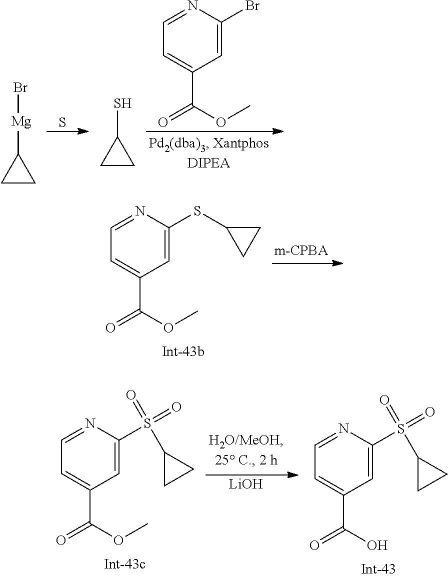

### Step 1: Preparation of cyclopropanethiol (Int-43a)

Sulfur (5.11 g, 160 mmol) was added to a solution of cyclopropylmagnesium bromide (0.5 M in THF, 320 mL, 160 mmol) at 0° C. Then the mixture was stirred at 50° C. for 3 h. After the reaction mixture was cooled to 0° C., lithium aluminum hydride (160 mmol) in THF was added and stirred at 65° C. for 0.5 h, quenched by H₂O (1 mL) at 0° C. then acidified by H₂SO₄ (5% v/v, 40 mL). The mixture was separated between water and CH₂Cl₂, the combined organic phase was dried and concentrated. The residue was used in the next step without further purification. MS obsd. (ESI⁺) [(M+H)⁺]: 75.1.

### Step 2: Preparation of methyl 2-cyclopropylsulfanylpyridine-4-carboxylate (Int-43b)

To a solution of methyl 2-bromoisonicotinate (160.0 mg, 0.7 mmol) in 1,4-dioxane (10 mL) was added cyclopropanethiol (148 mg, 2.0 mmol), DIEA (0.37 mL, 2.2 mmol) and Xantphos (85.7 mg, 0.15 mmol). Then the reaction mixture was purged with N₂ for three times. Tris(dibenzylideneacetone)dipalladium (0) (67.8 mg, 0.07 mmol) was added and the reaction was stirred at 80° C. for 1 h. LC-MS detection showed the reaction was completed. The reaction mixture was concentrated and the residue was purified by gel silica column to give methyl 2-cyclopropylsulfanylpyridine-4-carboxylate (137 mg). MS obsd. (ESI⁺) [(M+H)⁺]: 210.1.

### Step 3: Preparation of methyl 2-cyclopropylsulfonylpyridine-4-carboxylate (Int-43c)

To a solution of methyl 2-cyclopropylsulfanylpyridine-4-carboxylate (105.0 mg, 0.5 mmol) in CH₂Cl₂ (5 mL) was added m-CPBA (144.0 mg, 1.5 mmol). The mixture was stirred for 1 h at 25° C. Then LC-MS detection showed the reaction was completed. The mixture was washed by NaHCO₃ aqueous solution and the organic layer was concentrated to dryness. The residue was purified by flash column chromatography to afford methyl 2-cyclopropylsulfonylpyridine-4-carboxylate (93 mg). MS obsd. (ESI⁺) [(M+H)⁺]: 242.0.

### Step 4: Preparation of 2-cyclopropylsulfonylpyridine-4-carboxylic acid (Int-43)

To a solution of methyl 2-cyclopropylsulfanylpyridine-4-carboxylate (84 mg, 0.35 mmol) in methanol (5 mL) and water (5 mL) was added lithium hydroxide monohydrate (42 mg, 1.0 mmol) at 25° C., then the mixture was stirred for 2 h. LC-MS detection showed the reaction was completed. The mixture was concentrated to remove the MeOH, acidified to pH=2 with 1N HCl and extracted with CH₂Cl₂ (15 mL×3), the organic layer was dried and concentrated. The residue was used for the next step without further purification (57 mg). MS obsd. (ESI⁺) [(M+H)⁺]: 228.0.

### Intermediate Int-44

### 5-(Cyclopropylsulfonimidoyl)furan-2-carboxylic acid

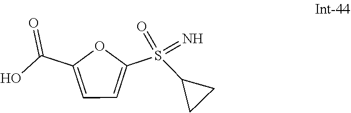 [SMILES: N=S(=O)(c1ccc(C(=O)O)o1)C1CC1]

The title compound was prepared in analogy to the procedure described for the preparation of 5-(methylsulfonimidoyl)furan-2-carboxylic acid (Int-33), by using methyl 5-cyclopropylsulfanylfuran-2-carboxylate instead of methyl 5-methylsulfanylfuran-2-carboxylate. MS obsd. (ESI⁺) [(M+H)⁺]: 216.1.

### Example 1

### N2-[[1-(5-Chloro-1,3-benzoxazol-2-yl)-4-piperidyl]methyl]furan-2,5-dicarboxamide

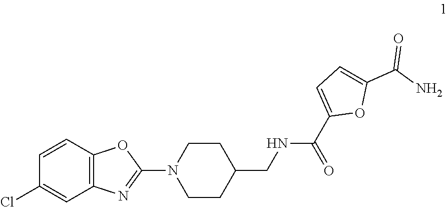 [SMILES: NC(=O)c1ccc(C(=O)NCC2CCN(c3nc4cc(Cl)ccc4o3)CC2)o1]

The title compound was prepared according to the following scheme:

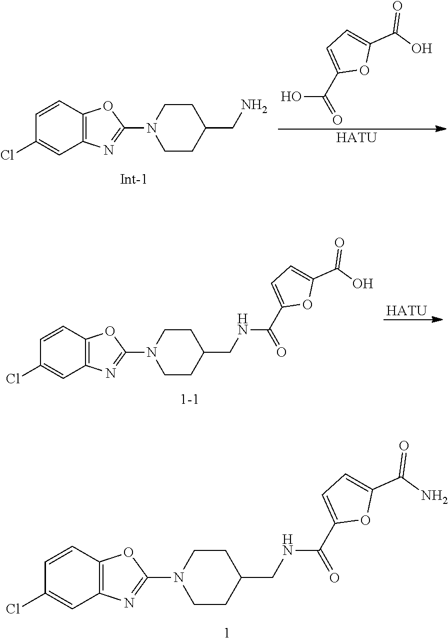

### Step 1: Preparation of 5-[[1-(5-chloro-1,3-benzoxazol-2-yl)-4-piperidyl]methylcarbamoyl]furan-2-carboxylic acid (1-1)

HATU (215 mg, 564 μmol) was added to a solution of TEA (190 mg, 262 μL, 1.88 mmol), (1-(5-chlorobenzo[d]oxazol-2-yl)piperidin-4-yl)methanamine 2,2,2-trifluoroacetic acid salt (Int-1,142 mg, 376 μmol, as the “AMINE” in Table 1) and furan-2,5-dicarboxylic acid (58.7 mg, 376 μmol, as the “ACID” or “SULFONYL CHLORIDE” in Table 1) in CH₂Cl₂ (10 ml) at r.t. Then the mixture was stirred at r.t. overnight. LC-MS detection indicated that the reaction was completed. The mixture was concentrated and the residue was purified by silica gel column to afford 5-[[1-(5-chloro-1,3-benzoxazol-2-yl)-4-piperidyl]methylcarbamoyl]furan-2-carboxylic acid (113 mg). MS obsd. (ESI⁺) [(M+H)⁺]: 404.1.

### Step 2: Preparation of N2-[[1-(5-chloro-1,3-benzoxazol-2-yl)-4-piperidyl]methy]furan-2,5-dicarboxamide (Example 1)

Ammonia (42.2 mg, 2.48 mmol) was added to a solution of HATU (141 mg, 371 μmol) and 5-(((1-(5-chlorobenzo[d]oxazol-2-yl)piperidin-4-yl)methyl)carbamoyl)furan-2-carboxylic acid (1-1, 100 mg, 248 μmol) in CH₂Cl₂ (10 mL) at r.t. Then the mixture was stirred at r.t. overnight. LC-MS detection indicated that the reaction was completed. The mixture was concentrated and the residue was purified by preparative HPLC to afford N2-[[1-(5-chloro-1,3-benzoxazol-2-yl)-4-piperidyl]methyl]furan-2,5-dicarboxamide (32 mg). MS obsd. (ESI⁺) [(M+H)⁺]: 403.1. ¹H NMR (400 MHz, CD₃OD) δ ppm: 7.33 (d, J=8.6 Hz, 1H), 7.27 (d, J=2.0 Hz, 1H), 7.23-7.20 (m, 1H), 7.19-7.18 (m, 1H), 7.08 (dd, J=2.1, 8.4 Hz, 1H), 4.29 (br d, J=13.2 Hz, 2H), 3.37 (d, J=6.6 Hz, 2H), 3.23 (dt, J=2.2, 12.8 Hz, 2H), 1.98-1.93 (m, 2H), 1.50-1.29 (m, 3H).

The following Example 2 to Example 20, Example 25 to Example 46, Example 49 to Example 62, Example 64 to Example 90, Example 93 to Example 122 were prepared in analogy to the procedure of Step-1 described for the preparation of Example 1, replacing Int-1 with “AMINE”, and replacing furan-2,5-dicarboxylic acid with “ACID” or “SULFONYL CHLORIDE”. The “AMINE”, “ACID” and “SULFONYL CHLORIDE” are the reagents indicated in Table 1.

TABLE 1
Compounds synthesis and characterization

| Example | | AMINE & | |
|---|---|---|---|
| No. | Compound Name and Structure | ACID | ¹H NMR and MS (ESI⁺) |
| 2 | 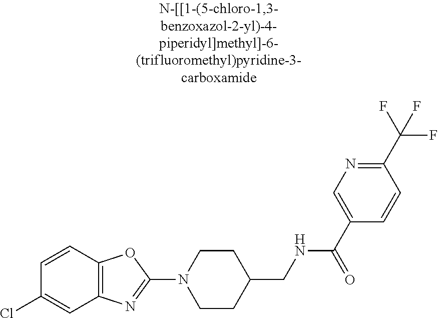 [SMILES: O=C(NCC1CCN(c2nc3cc(Cl)ccc3o2)CC1)c1ccc(C(F)(F)F)nc1] | AMINE: Int-1 ACID: 6- (trifluoromethyl) pyridine-3- carboxylic acid | ¹H NMR (400 MHz, CD₃OD) δ ppm: 9.12 (d, J = 2.0 Hz, 1H), 8.43 (dd, J = 1.7, 8.1 Hz, 1H), 7.95 (d, J = 8.2 Hz, 1H), 7.29 (d, J = 8.4 Hz, 1H), 7.24 (d, J = 2.1 Hz, 1H), 7.04 (dd, J = 2.1, 8.5 Hz, 1H), 4.30 (br d, J = 13.3 Hz, 2H), 3.39 (d, J = 6.7 Hz, 2H), 3.20 (dt, J = 2.7, 12.8 Hz, 2H), 2.09-1.91 (m, 4H), 1.49-1.41 (m, 1H). MS obsd. (ESI⁺) [(M + H)⁺]: 439.1. |
| 3 | 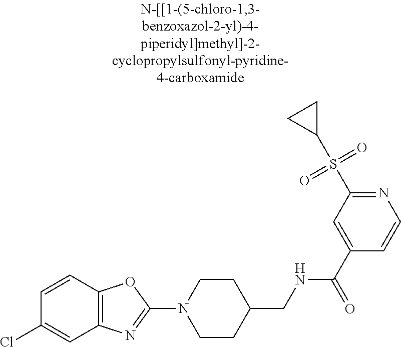 [SMILES: O=C(NCC1CCN(c2nc3cc(Cl)ccc3o2)CC1)c1ccnc(S(=O)(=O)C2CC2)c1] | AMINE: Int-1 ACID: Int-43 | ¹H NMR (400 MHz, CD₃OD) δ ppm: 8.91 (d, J = 4.9 Hz, 1H), 8.39 (d, J = 0.7 Hz, 1H), 8.05 (dd, J = 1.7, 5.0 Hz, 1H), 7.28 (d, J = 8.6 Hz, 1H), 7.23 (d, J = 2.1 Hz, 1H), 7.03 (dd, J = 2.1, 8.4 Hz, 1H), 4.27 (br d, J = 13.2 Hz, 2H), 3.38 (t, J = 6.4 Hz, 2H), 3.19 (dt, J = 2.6, 12.8 Hz, 2H), 2.98-2.88 (m, 1H), 2.04- 1.88 (m, 3H), 1.40 (dq, J = 4.3, 12.3 Hz, 2H), 1.32-1.27 (m, 2H), 1.17-1.11 (m, 2H). MS obsd. (ESI⁺) [(M + H)⁺]: 475.1 |
| 4 | 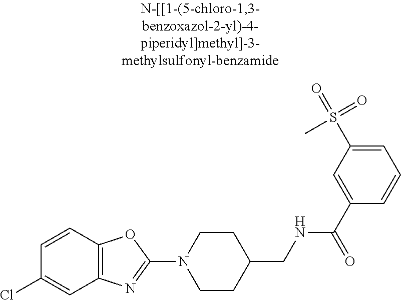 [SMILES: CS(=O)(=O)c1cccc(C(=O)NCC2CCN(c3nc4cc(Cl)ccc4o3)CC2)c1] | AMINE: Int-1 ACID: 3- methylsulfonyl benzoic acid | ¹H NMR (400 MHz, CD₃OD) δ ppm: 8.42 (t, J = 1.7 Hz, 1H), 8.20-8.13 (m, 2H), 7.77 (t, J = 7.8 Hz, 1H), 7.29 (d, J = 8.4 Hz, 1H), 7.24 (d, J = 2.1 Hz, 1H), 7.03 (dd, J = 2.1, 8.4 Hz, 1H), 4.30 (br d, J = 13.2 Hz, 2H), 3.38 (d, J = 6.8 Hz, 2H), 3.25- 3.14 (m, 5H), 2.05-1.89 (m, 3H), 1.47-1.36 (m, 2H). MS obsd. (ESI⁺) [(M + H)⁺]: 448.1 |
| 5 | 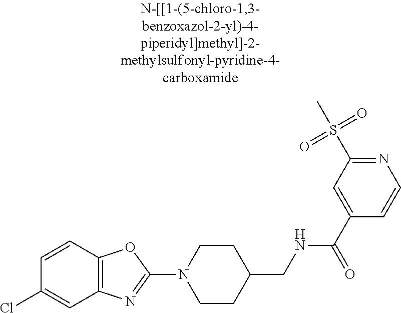 [SMILES: CS(=O)(=O)c1cc(C(=O)NCC2CCN(c3nc4cc(Cl)ccc4o3)CC2)ccn1] | AMINE: Int-1 ACID: Int-41 | ¹H NMR (400 MHz, CD₃OD) δ ppm: 8.91 (dd, J = 0.7, 5.0 Hz, 1H), 8.46 (dd, J = 0.8, 1.5 Hz, 1H), 8.06 (dd, J = 1.6, 5.0 Hz, 1H), 7.29 (d, J = 8.6 Hz, 1H), 7.24 (d, J = 2.1 Hz, 1H), 7.03 (dd, J = 2.1, 8.4 Hz, 1H), 4.30 (br d, J = 13.2 Hz, 2H), 3.39 (d, J = 6.8 Hz, 2H), 3.29 (s, 3H), 3.19 (dt, J = 2.6, 12.9 Hz, 2H), 2.04-1.89 (m, 3H), 1.48-1.36 (m, 2H) MS obsd. (ESI⁺) [(M + H)⁺]: 449.1 |
| 6 | 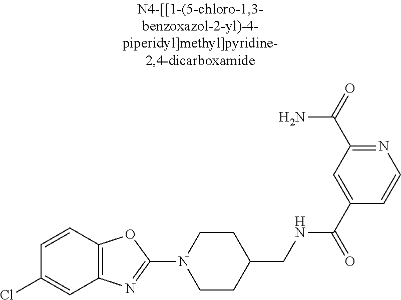 [SMILES: NC(=O)c1cc(C(=O)NCC2CCN(c3nc4cc(Cl)ccc4o3)CC2)ccn1] | AMINE: Int-1 ACID: Int-22 | ¹H NMR (400 MHz, CD₃OD) δ ppm: 8.67 (dd, J = 0.7, 5.0 Hz, 1H), 8.37 (dd, J = 0.7, 1.7 Hz, 1H), 7.80 (dd, J = 1.7, 5.0 Hz, 1H), 7.17 (d, J = 8.4 Hz, 1H), 7.12 (d, J = 2.1 Hz, 1H), 6.91 (dd, J = 2.1, 8.5 Hz, 1H), 4.18 (br d, J = 13.1 Hz, 2H), 3.26 (d, J = 6.8 Hz, 2H), 3.07 (dt, J = 2.8, 12.9 Hz, 2H), 1.97-1.79 (m, 3H), 1.36-1.25 (m, 2H). MS obsd. (ESI⁺) [(M + H)⁺]: 414.1 |
| 7 | 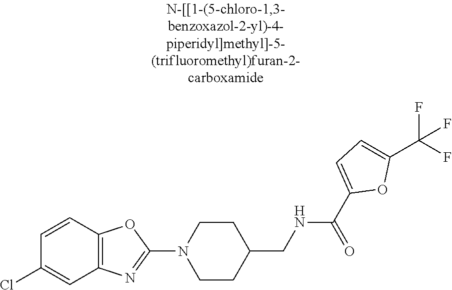 [SMILES: O=C(NCC1CCN(c2nc3cc(Cl)ccc3o2)CC1)c1ccc(C(F)(F)F)o1] | AMINE: Int-1 ACID: 5- (trifluoromethyl) furan-2- carboxylic acid | ¹H NMR (400 MHz, CD₃OD) δ ppm: 7.29 (d, J = 8.4 Hz, 1H), 7.25-7.21 (m, 2H), 7.18-7.14 (m, 1H), 7.03 (dd, J = 2.1, 8.5 Hz, 1H), 4.28 (br d, J = 13.3 Hz, 2H), 3.18 (dt, J = 2.8, 12.9 Hz, 2H), 2.03-1.87 (m, 3H), 1.46- 1.29 (m, 4H). MS obsd. (ESI⁺) [(M + H)⁺]: 428.1 |
| 8 | 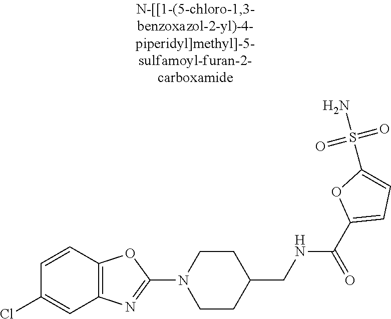 [SMILES: NS(=O)(=O)c1ccc(C(=O)NCC2CCN(c3nc4cc(Cl)ccc4o3)CC2)o1] | AMINE: Int-1 Acid: 5- sulfamoylfuran- 2-carboxylic acid | ¹H NMR (400 MHz, CD₃OD) δ ppm: 7.32 (d, J = 8.4 Hz, 1H), 7.27 (d, J = 2.0 Hz, 1H), 7.18 (d, J = 3.5 Hz, 1H), 7.11-7.06 (m, 2H), 4.28 (br d, J = 13.3 Hz, 2H), 3.22 (dt, J = 2.7, 12.8 Hz, 2H), 2.25-2.19 (m, 1H), 2.08- 1.90 (m, 4H), 1.45-1.38 (m, 2H). MS obsd. (ESI⁺) [(M + H)⁺]: 439.1 |
| 9 | 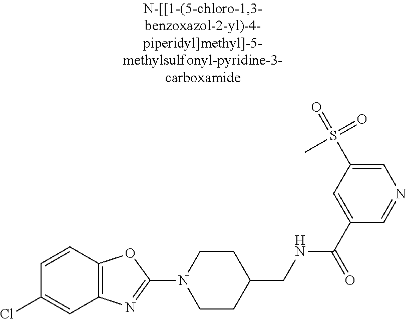 [SMILES: CS(=O)(=O)c1cncc(C(=O)NCC2CCN(c3nc4cc(Cl)ccc4o3)CC2)c1] | AMINE: Int-1 ACID: 5- methylsulfonyl pyridine-3- carboxylic acid | ¹H NMR (400 MHz, CD₃OD) δ ppm: 9.29 (d, J = 2.0 Hz, 1H), 9.24 (d, J = 2.1 Hz, 1H), 8.74 (t, J = 2.1 Hz, 1H), 7.29 (d, J = 8.4 Hz, 1H), 7.24 (d, J = 2.1 Hz, 1H), 7.04 (dd, J = 2.1, 8.5 Hz, 1H), 4.34-4.27 (m, 2H), 3.40 (d, J = 6.8 Hz, 2H), 3.28 (s, 3H), 3.25-3.16 (m, 2H), 2.20 (d, J = 7.7 Hz, 1H), 1.98-1.91 (m, 2H), 1.48-1.40 (m, 2H). MS obsd. (ESI⁺) [(M + H)⁺]: 449.1. |
| 10 | 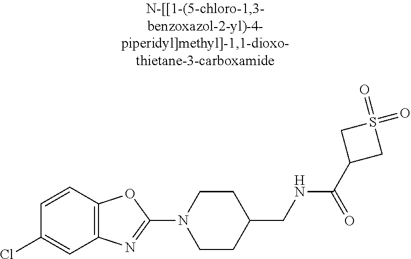 [SMILES: O=C(NCC1CCN(c2nc3cc(Cl)ccc3o2)CC1)C1CS(=O)(=O)C1] | AMINE: Int-1 ACID: 1,1- dioxothietane- 3-carboxylic acid | ¹H NMR (400 MHz, CD₃OD) δ ppm: 7.29 (d, J = 8.4 Hz, 1H), 7.24 (d, J = 2.0 Hz, 1H), 7.03 (dd, J = 2.1, 8.4 Hz, 1H), 4.42- 4.34 (m, 2H), 4.31-4.23 (m, 4H), 3.22-3.12 (m, 4H), 1.90- 1.82 (m, 3H), 1.39-1.26 (m, 3H). MS obsd. (ESI⁺) [(M + H)⁺]: 398.1. |
| 11 | 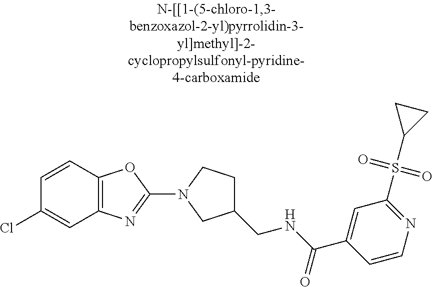 [SMILES: O=C(NCC1CCN(c2nc3cc(Cl)ccc3o2)C1)c1ccnc(S(=O)(=O)C2CC2)c1] | AMINE: Int-2 Acide: Int-43 | ¹H NMR (400 MHz, CD₃OD) δ ppm: 8.92 (dd, J = 0.7, 5.0 Hz, 1H), 8.41 (dd, J = 0.7, 1.6 Hz, 1H), 8.06 (dd, J = 1.7, 5.0 Hz, 1H), 7.30 (d, J = 8.4 Hz, 1H), 7.24 (d, J = 2.1 Hz, 1H), 7.04 (dd, J = 2.1, 8.5 Hz, 1H), 3.89- 3.77 (m, 2H), 3.68 (td, J = 7.6, 10.4 Hz, 1H), 3.61-3.46 (m, 3H), 2.94 (tt, J = 4.8, 8.0 Hz, 1H), 2.84-2.73 (m, 1H), 2.32- 2.22 (m, 1H), 2.01-1.91 (m, 1H), 1.33-1.27 (m, 2H), 1.18- 1.11 (m, 2H). MS obsd. (ESI⁺) [(M + H)⁺]: 461.1. |
| 12 | 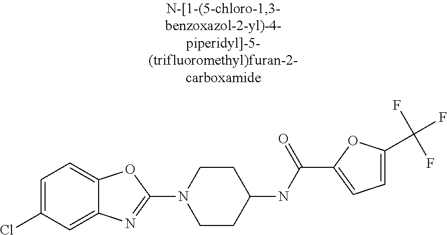 [SMILES: O=C(NC1CCN(c2nc3cc(Cl)ccc3o2)CC1)c1ccc(C(F)(F)F)o1] | AMINE: Int-3 ACID: 5- (trifluoromethyl) furan-2- carboxylic acid | ¹H NMR (400 MHz, DMSO-d₆) δ ppm: 8.60 (d, J = 8.0 Hz, 1H), 7.43 (d, J = 8.5 Hz, 1H), 7.38- 7.36 (m, 1H), 7.34 (d, J = 2.1 Hz, 1H), 7.30 (d, J = 3.2 Hz, 1H), 7.04 (dd, J = 8.5, 2.2 Hz, 1H), 4.17 (s, 1H), 4.14 (s, 1H), 4.12-4.04 (m, 1H), 3.31-3.24 (m, 2H), 1.91 (dd, J = 12.6, 2.7 Hz, 2H), 1.69-1.59 (m, 2H). MS obsd. (ESI⁺) [(M + H)⁺]: 414.1. |
| 13 | 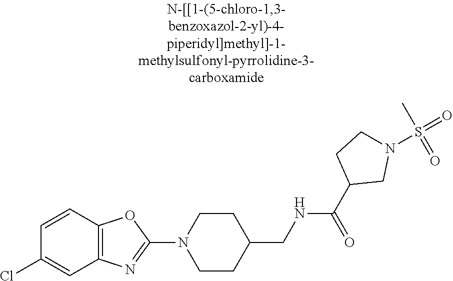 [SMILES: CS(=O)(=O)N1CCC(C(=O)NCC2CCN(c3nc4cc(Cl)ccc4o3)CC2)C1] | AMINE: Int-4 SULFONYL CHLORIDE: methanesulfonyl chloride | ¹H NMR (400 MHz, CD₃OD) δ ppm: 7.28 (d, J = 8.6 Hz, 1H), 7.24 (d, J = 2.1 Hz, 1H), 7.03 (dd, J = 2.1, 8.5 Hz, 1H), 4.31- 4.24 (m, 2H), 3.59-3.53 (m, 1H), 3.50-3.43 (m, 2H), 3.41- 3.36 (m, 1H), 3.21-3.05 (m, 5H), 2.92 (s, 3H), 2.26-2.10 (m, 2H), 1.87 (br d, J = 1.8 Hz, 2H), 1.40-1.30 (m, 3H). MS obsd. (ESI⁺) [(M + H)⁺]: 441.1. |
| 14 | 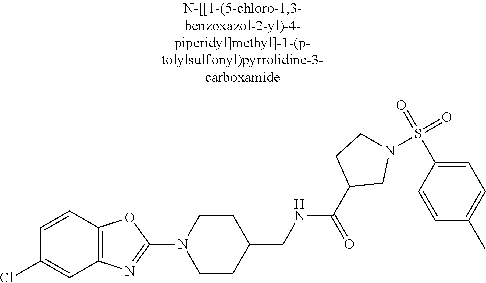 [SMILES: Cc1ccc(S(=O)(=O)N2CCC(C(=O)NCC3CCN(c4nc5cc(Cl)ccc5o4)CC3)C2)cc1] | AMINE: Int-4 SULFONYL CHLORIDE: 4- methylbenzene sulfonyl chloride | ¹H NMR (400 MHz, CD₃OD) δ ppm: 7.74 (d, J = 8.2 Hz, 2H), 7.44 (d, J = 8.1 Hz, 2H), 7.28 (d, J = 8.4 Hz, 1H), 7.23 (d, J = 2.1 Hz, 1H), 7.03 (dd, J = 2.1, 8.4 Hz, 1H), 4.25 (br d, J = 13.2 Hz, 2H), 3.53 (dd, J = 8.1, 9.8 Hz, 1H), 3.40-3.35 (m, 1H), 3.31- 3.24 (m, 2H), 3.18-3.05 (m, 4H), 2.88 (quin, J = 7.9 Hz, 1H), 2.46 (s, 3H), 2.08-1.88 (m, 2H), 1.82-1.76 (m, 2H), 1.36- 1.26 (m, 3H). MS obsd. (ESI⁺) [(M + H)⁺]: 517.2. |
| 15 | 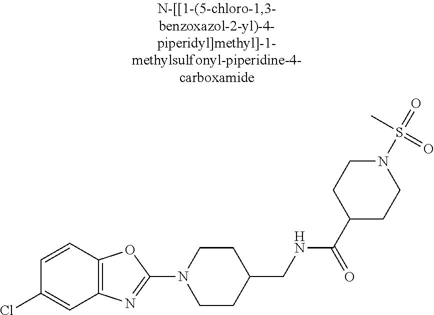 [SMILES: CS(=O)(=O)N1CCC(C(=O)NCC2CCN(c3nc4cc(Cl)ccc4o3)CC2)CC1] | AMINE: Int-5 SULFONYL CHLORIDE: methanesulfonyl chloride | ¹H NMR (400 MHz, CD₃OD) δ ppm: 7.29 (d, J = 8.4 Hz, 1H), 7.24 (d, J = 2.1 Hz, 1H), 7.03 (dd, J = 2.1, 8.4 Hz, 1H), 4.27 (br d, J = 13.1 Hz, 2H), 3.83- 3.72 (m, 2H), 3.19-3.11 (m, 4H), 2.85 (s, 3H), 2.83-2.67 (m, 2H), 2.35 (s, 1H), 1.91- 1.76 (m, 6H), 1.34 (br dd, J = 2.8, 4.8 Hz, 3H). MS obsd. (ESI⁺) [(M + H)⁺]: 455.1. |
| 16 | 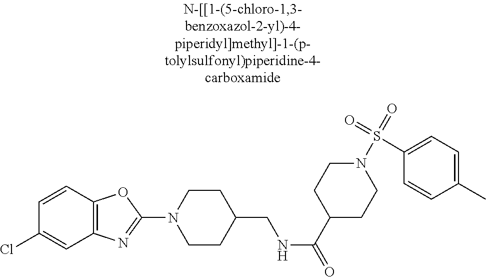 [SMILES: Cc1ccc(S(=O)(=O)N2CCC(C(=O)NCC3CCN(c4nc5cc(Cl)ccc5o4)CC3)CC2)cc1] | AMINE: Int-5 SULFONYL CHLORIDE: 4- methylbenzene sulfonyl chloride | ¹H NMR (400 MHz, CD₃OD) δ ppm: 7.67 (d, J = 8.3 Hz, 2H), 7.44 (d, J = 8.2 Hz, 2H), 7.28 (d, J = 8.4 Hz, 1H), 7.23 (d, J = 2.0 Hz, 1H), 7.03 (dd, J = 2.1, 8.4 Hz, 1H), 4.24 (br d, J = 13.2 Hz, 2H), 3.79-3.71 (m, 2H), 3.17- 3.08 (m, 4H), 2.46 (s, 3H), 2.34 (dt, J = 3.0, 11.6 Hz, 2H), 2.24- 2.08 (m, 1H), 1.84-1.74 (m, 6H), 1.36-1.28 (m, 3H). MS obsd. (ESI⁺) [(M + H)⁺]: 531.2. |
| 17 | 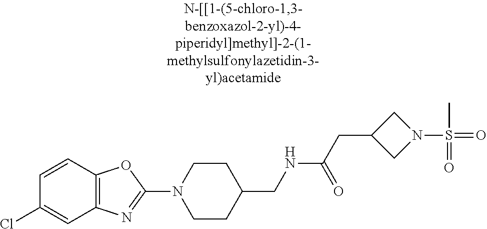 [SMILES: CS(=O)(=O)N1CC(CC(=O)NCC2CCN(c3nc4cc(Cl)ccc4o3)CC2)C1] | AMINE: Int-6 SULFONYL CHLORIDE: methanesulfonyl chloride | ¹H NMR (400 MHz, CD₃OD) δ ppm: 7.28 (d, J = 8.6 Hz, 1H), 7.24 (d, J = 2.1 Hz, 1H), 7.03 (dd, J = 2.1, 8.4 Hz, 1H), 4.33- 4.22 (m, 2H), 4.04 (t, J = 8.2 Hz, 2H), 3.69 (dd, J = 6.2, 8.0 Hz, 2H), 3.23-3.08 (m, 4H), 3.03- 2.90 (m, 4H), 2.57 (d, J = 7.6 Hz, 2H), 1.90-1.81 (m, 3H), 1.39-1.32 (m, 2H). MS obsd. (ESI⁺) [(M + H)⁺]: 441.1. |
| 18 | 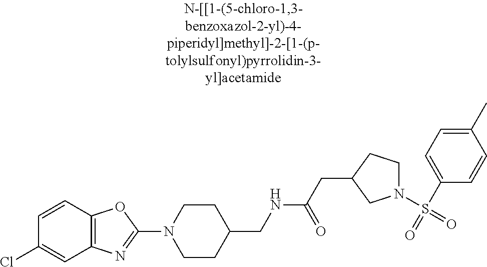 [SMILES: Cc1ccc(S(=O)(=O)N2CCC(CC(=O)NCC3CCN(c4nc5cc(Cl)ccc5o4)CC3)C2)cc1] | AMINE: Int-7 SULFONYL CHLORIDE: 4- methylbenzene sulfonyl chloride | ¹H NMR (400 MHz, CD₃OD) δ ppm: 7.72 (d, J = 8.3 Hz, 2H), 7.44 (d, J = 7.9 Hz, 2H), 7.29 (d, J = 8.4 Hz, 1H), 7.24 (d, J = 2.0 Hz, 1H), 7.03 (dd, J = 2.1, 8.5 Hz, 1H), 4.32-4.23 (m, 2H), 3.42 (dd, J = 7.2, 9.9 Hz, 1H), 3.21-3.10 (m, 5H), 2.90 (dd, J = 7.6, 9.9 Hz, 1H), 2.46 (s, 3H), 2.40 (td, J = 7.6, 15.0 Hz, 1H), 2.22-2.17 (m, 2H), 1.99-1.90 (m, 1H), 1.87-1.80 (m, 2H), 1.46 (dd, J = 8.3, 12.5 Hz, 1H), 1.35 (m, 4H). MS obsd. (ESI⁺) [(M + H)⁺]: 531.2. |
| 19 | 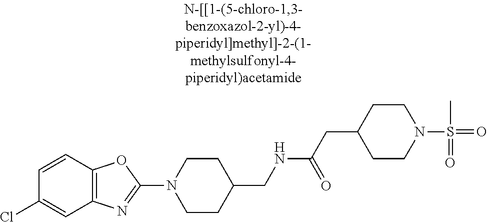 [SMILES: CS(=O)(=O)N1CCC(CC(=O)NCC2CCN(c3nc4cc(Cl)ccc4o3)CC2)CC1] | AMINE: Int-8 SULFONYL CHLORIDE: methanesulfonyl chloride | ¹H NMR (400 MHz, CD₃OD) δ ppm: 7.29 (d, J = 8.6 Hz, 1H), 7.24 (d, J = 2.1 Hz, 1H), 7.04 (dd, J = 2.1, 8.5 Hz, 1H), 4.32- 4.24 (m, 2H), 3.70 (br d, J = 11.9 Hz, 2H), 3.20-3.11 (m, 4H), 2.82 (s, 3H), 2.73 (dt, J = 2.6, 12.1 Hz, 2H), 2.19 (d, J = 7.2 Hz, 2H), 1.88-1.80 (m, 4H), 1.37-1.33 (m, 5H). MS obsd. (ESI⁺) [(M + H)⁺]: 469.2. |
| 20 | 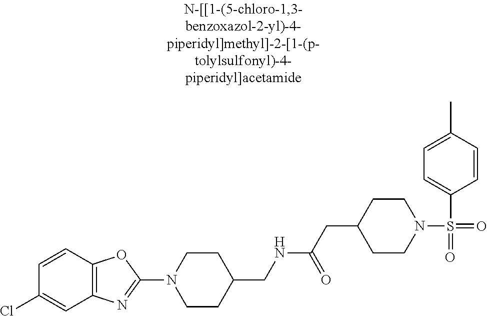 [SMILES: Cc1ccc(S(=O)(=O)N2CCC(CC(=O)NCC3CCN(c4nc5cc(Cl)ccc5o4)CC3)CC2)cc1] | AMINE: Int-8 SULFONYL CHLORIDE: 4- methylbenzene sulfonyl chloride | ¹H NMR (400 MHz, CD₃OD) δ ppm: 7.65 (d, J = 8.3 Hz, 2H), 7.43 (d, J = 8.1 Hz, 2H), 7.29 (d, J = 8.6 Hz, 1H), 7.24 (d, J = 2.1 Hz, 1H), 7.04 (dd, J = 2.1, 8.4 Hz, 1H), 4.31-4.21 (m, 2H), 3.72 (br d, J = 11.7 Hz, 2H), 3.20-3.10 (m, 4H), 2.46 (s, 3H), 2.32-2.19 (m, 3H), 2.12 (d, J = 7.0 Hz, 2H), 1.86-1.80 (m, 2H), 1.77-1.70 (m, 2H), 1.67-1.59 (m, 1H), 1.35 (m, 4H). MS obsd. (ESI⁺) [(M + H)⁺]: 545.2. |
| 25 | 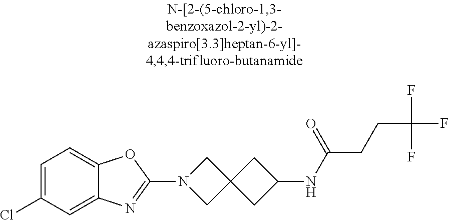 [SMILES: O=C(CCC(F)(F)F)NC1CC2(C1)CN(c1nc3cc(Cl)ccc3o1)C2] | AMINE: Int-9 ACID: 4,4,4- trifluorobutanoic acid | ¹H NMR (400 MHz, DMSO-d₆) δ ppm: 8.27 (br d, J = 7.0 Hz, 1H), 7.42 (br d, J = 8.4 Hz, 1H), 7.33 (s, 1H), 7.07-7.02 (m, 1H), 4.26 (s, 2H), 4.15 (s, 2H), 4.13-4.02 (m, 1H), 2.54 (br s, 2H), 2.48-2.40 (m, 2H), 2.32 (br d, J = 7.8 Hz, 2H), 2.12 (br s, 2H). MS obsd. (ESI⁺) [(M + H)⁺]: 388.1. |
| 26 | 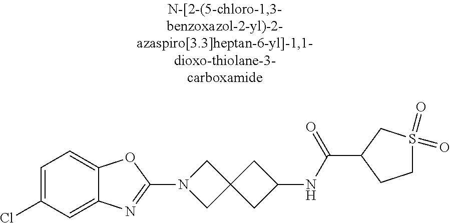 [SMILES: O=C(NC1CC2(C1)CN(c1nc3cc(Cl)ccc3o1)C2)C1CCS(=O)(=O)C1] | AMINE: Int-9 ACID: 1,1- dioxothiolane- 3-carboxylic acid | ¹H NMR (400 MHz, DMSO-d₆) δ ppm: 8.38 (br d, J = 7.1 Hz, 1H), 7.42 (d, J = 8.6 Hz, 1H), 7.33 (d, J = 1.8 Hz, 1H), 7.05 (dd, J = 1.8, 8.3 Hz, 1H), 4.26 (s, 2H), 4.16 (s, 2H), 4.12-4.04 (m, 1H), 3.30-3.18 (m, 2H), 3.16-3.02 (m, 3H), 2.57 (br d, J = 7.8 Hz, 2H), 2.37-2.26 (m, 1H), 2.19-2.00 (m, 3H). MS obsd. (ESI⁺) [(M + H)⁺]: 410.1. |
| 27 | 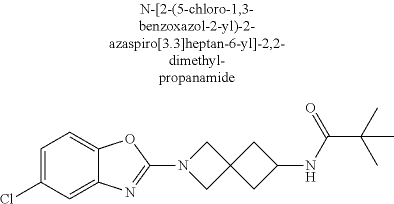 [SMILES: CC(C)(C)C(=O)NC1CC2(C1)CN(c1nc3cc(Cl)ccc3o1)C2] | AMINE: Int-9 ACID: 2,2- dimethyl- propanoic acid | ¹H NMR (400 MHz, DMSO-d₆) δ ppm: 7.55 (d, J = 7.3 Hz, 1H), 7.42 (d, J = 8.5 Hz, 1H), 7.33 (d, J = 2.1 Hz, 1H), 7.04 (dd, J = 8.5, 2.2 Hz, 1H), 4.26 (s, 2H), 4.18-4.05 (m, 3H), 2.53-2.48 (m, 2H), 2.22 (dd, J = 12.3, 8.9 Hz, 2H), 1.07 (s, 9H). MS obsd. (ESI⁺) [(M + H)⁺]: 348.1. |
| 28 | 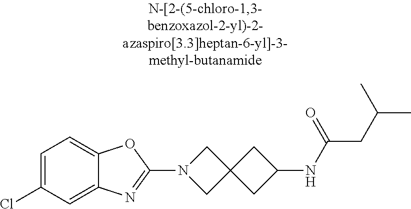 [SMILES: CC(C)CC(=O)NC1CC2(C1)CN(c1nc3cc(Cl)ccc3o1)C2] | AMINE: Int-9 ACID: 3- methylbutanoic acid | ¹H NMR (400 MHz, DMSO-d₆) δ ppm: 8.01 (d, J = 7.3 Hz, 1H), 7.42 (d, J = 8.5 Hz, 1H), 7.33 (d, J = 2.1 Hz, 1H), 7.04 (dd, J = 8.5, 2.2 Hz, 1H), 4.26 (s, 2H), 4.17-4.03 (m, 3H), 2.61-2.52 (m, 2H), 2.14-2.06 (m, 2H), 1.97-1.85 (m, 3H), 0.85 (d, J = 6.3 Hz, 6H). MS obsd. (ESI⁺) [(M + H)⁺]: 348.1. |
| 29 | 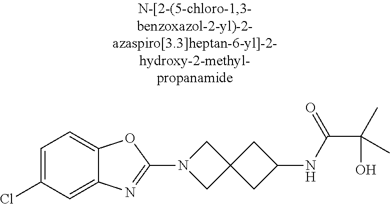 [SMILES: CC(C)(O)C(=O)NC1CC2(C1)CN(c1nc3cc(Cl)ccc3o1)C2] | AMINE: Int-9 ACID: 2- hydroxy-2- methyl- propanoic acid | ¹H NMR (400 MHz, CDCl₃) δ ppm: 7.34 (d, J = 2.0 Hz, 1H), 7.16 (d, J = 8.5 Hz, 1H), 7.01 (dd, J = 8.5, 2.1 Hz, 1H), 6.88 (d, J = 7.2 Hz, 1H), 4.37-4.21 (m, 6H), 2.79-2.68 (m, 2H), 2.27-2.16 (m, 2H), 1.45 (s, 6H). MS obsd. (ESI⁺) [(M + H)⁺]: 352.1. |
| 30 | 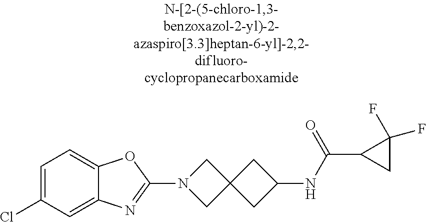 [SMILES: O=C(NC1CC2(C1)CN(c1nc3cc(Cl)ccc3o1)C2)C1CC1(F)F] | AMINE: Int-9 ACID: 2,2- difluorocyclop ropanecarboxy lic acid | ¹H NMR (400 MHz, CD₃OD) δ ppm: 7.25 (dd, J = 13.9, 5.3 Hz, 2H), 7.03 (dd, J = 8.5, 2.1 Hz, 1H), 4.32 (s, 2H), 4.27-4.14 (m, 3H), 2.68 (dd, J = 11.7, 7.3 Hz, 2H), 2.44 (ddd, J = 13.5, 10.9, 7.8 Hz, 1H), 2.28-2.18 (m, 2H), 1.96 (ddd, J = 12.8, 7.6, 1.6 Hz, 1H), 1.78-1.66 (m, 1H). MS obsd. (ESI⁺) [(M + H)⁺]: 368.1. |
| 31 | 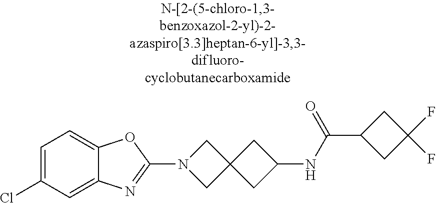 [SMILES: O=C(NC1CC2(C1)CN(c1nc3cc(Cl)ccc3o1)C2)C1CC(F)(F)C1] | AMINE: Int-9 ACID: 3,3- difluorocyclo- butanecarboxylic acid | ¹H NMR (400 MHz, DMSO-d₆) δ ppm: 8.28 (d, J = 7.3 Hz, 1H), 7.42 (d, J = 8.5 Hz, 1H), 7.33 (d, J = 2.1 Hz, 1H), 7.04 (dd, J = 8.5, 2.2 Hz, 1H), 4.26 (s, 2H), 4.22-4.00 (m, 3H), 2.84-2.74 (m, 1H), 2.66 (dt, J = 17.3, 8.6 Hz, 4H), 2.58-2.52 (m, 2H), 2.20-2.06 (m, 2H). MS obsd. (ESI⁺) [(M + H)⁺]: 382.1. |
| 32 | 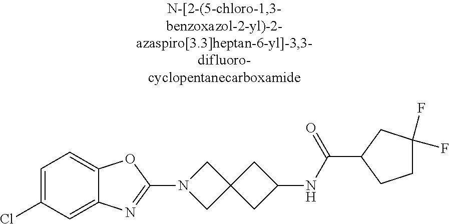 [SMILES: O=C(NC1CC2(C1)CN(c1nc3cc(Cl)ccc3o1)C2)C1CCC(F)(F)C1] | AMINE: Int-9 ACID: 3,3- difluorocyclo- pentane- carboxylic acid | ¹H NMR (400 MHz, DMSO-d₆) δ ppm: 8.19 (d, J = 7.2 Hz, 1H), 7.42 (d, J = 8.5 Hz, 1H), 7.33 (d, J = 2.1 Hz, 1H), 7.04 (dd, J = 8.5, 2.1 Hz, 1H), 4.26 (s, 2H), 4.15 (s, 2H), 4.08 (dd, J = 15.6, 7.9 Hz, 1H), 2.85-2.74 (m, 1H), 2.60-2.52 (m, 2H), 2.26-2.08 (m, 5H), 2.06-1.89 (m, 2H), 1.77 (dd, J = 12.6, 8.7 Hz, 1H). MS obsd. (ESI⁺) [(M + H)⁺]: 396.1. |
| 33 | 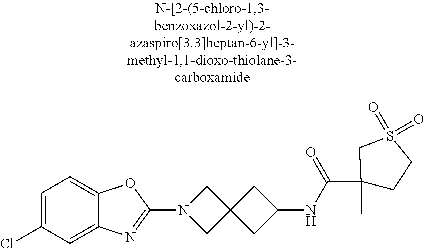 [SMILES: CC1(C(=O)NC2CC3(C2)CN(c2nc4cc(Cl)ccc4o2)C3)CCS(=O)(=O)C1] | AMINE: Int-9 ACID: 3- methyl-1,1- dioxo-thiolane- 3-carboxylic acid | ¹H NMR (400 MHz, DMSO-d₆) δ ppm: 8.03 (d, J = 6.8 Hz, 1H), 7.42 (d, J = 8.6 Hz, 1H), 7.33 (d, J = 2.1 Hz, 1H), 7.05 (dd, J = 8.4, 2.2 Hz, 1H), 4.27 (s, 2H), 4.17 (s, 2H), 4.05-4.14 (m, 1H), 3.61 (d, J = 13.4 Hz, 1H), 3.16- 3.27 (m, 1H), 2.95-3.09 (m, 2H), 2.52-2.59 (m, 2H), 2.42-2.49 (m, 1H), 2.22 (dd, J = 12.7, 8.4 Hz, 2H), 2.03 (dt, J = 13.7, 7.8 Hz, 1H), 1.33-1.40 (m, 3H). MS obsd. (ESI⁺) [(M + H)⁺]: 424.1. |
| 34 | 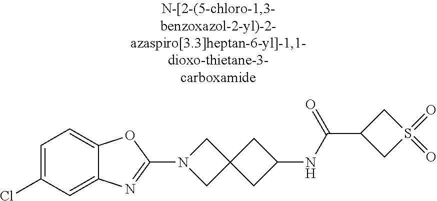 [SMILES: O=C(NC1CC2(C1)CN(c1nc3cc(Cl)ccc3o1)C2)C1CS(=O)(=O)C1] | AMINE: Int-9 ACID: 1,1- dioxothietane- 3-carboxylic acid | ¹H NMR (400 MHz, CDCl₃) δ ppm: 7.35 (d, J = 2.0 Hz, 1H), 7.17 (d, J = 8.5 Hz, 1H), 7.03 (dd, J = 8.5, 2.0 Hz, 1H), 6.09- 5.99 (m, 1H), 4.51-4.13 (m, 9H), 3.15 (dd, J = 17.4, 8.9 Hz, 1H), 2.80-2.69 (m, 2H), 2.30- 2.19 (m, 2H). MS obsd. (ESI⁺) [(M + H)⁺]: 396.1. |
| 35 | 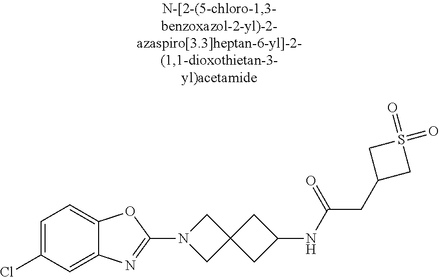 [SMILES: O=C(CC1CS(=O)(=O)C1)NC1CC2(C1)CN(c1nc3cc(Cl)ccc3o1)C2] | AMINE: Int-9 ACID: 2-(1,1- dioxothietan-3- yl)acetic acid | ¹H NMR (400 MHz, DMSO-d₆) δ ppm: 8.19 (d, J = 7.2 Hz, 1H), 7.42 (d, J = 8.5 Hz, 1H), 7.33 (d, J = 2.1 Hz, 1H), 7.04 (dd, J = 8.5, 2.1 Hz, 1H), 4.26 (s, 2H), 4.15 (s, 2H), 4.08 (dd, J = 15.6, 7.9 Hz, 1H), 2.85-2.74 (m, 1H), 2.60-2.52 (m, 2H), 2.26-2.08 (m, 5H), 2.06-1.89 (m, 2H), 1.77 (dd, J = 12.6, 8.7 Hz, 1H). MS obsd. (ESI⁺) [(M + H)⁺]: 432.1. |
| 36 | 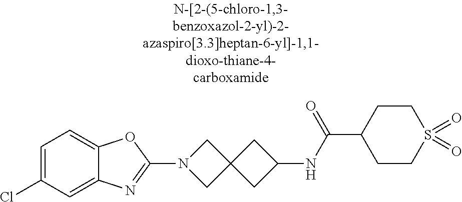 [SMILES: O=C(NC1CC2(C1)CN(c1nc3cc(Cl)ccc3o1)C2)C1CCS(=O)(=O)CC1] | AMINE: Int-9 ACID: 1,1- dioxothiane-4- carboxylic acid | ¹H NMR (400 MHz, DMSO-d₆) δ ppm: 8.19 (d, J = 7.2 Hz, 1H), 7.42 (d, J = 8.5 Hz, 1H), 7.33 (d, J = 2.1 Hz, 1H), 7.04 (dd, J = 8.5, 2.1 Hz, 1H), 4.20 (d, J = 41.1 Hz, 4H), 4.11-4.01 (m, 2H), 3.12 (ddd, J = 22.1, 14.7, 8.9 Hz, 4H), 2.59-2.54 (m, 1H), 2.38 (qd, J = 10.5, 5.3 Hz, 1H), 2.19-1.89 (m, 6H). MS obsd. (ESI⁺) [(M + H)⁺]: 424.1. |
| 37 | 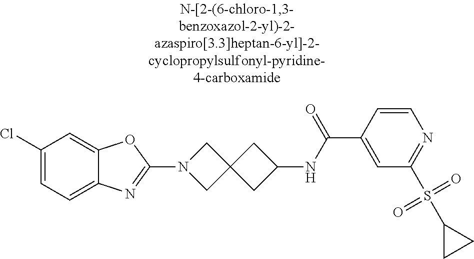 [SMILES: O=C(NC1CC2(C1)CN(c1nc3ccc(Cl)cc3o1)C2)c1ccnc(S(=O)(=O)C2CC2)c1] | AMINE: Int- 11 ACID: Int-43 | ¹H NMR (400 MHz, DMSO-d₆) δ ppm: 9.23 (d, J = 7.1 Hz, 1H), 8.96 (d, J = 4.9 Hz, 1H), 8.38 (s, 1H), 8.09 (dd, J = 4.9, 1.5 Hz, 1H), 7.59 (d, J = 1.9 Hz, 1H), 7.28 (d, J = 8.4 Hz, 1H), 7.20 (dd, J = 8.4, 2.0 Hz, 1H), 4.43- 4.27 (m, 3H), 4.20 (s, 2H), 3.06- 2.94 (m, 1H), 2.69-2.62 (m, 2H), 2.41-2.34 (m, 2H), 1.19- 1.07 (m, 4H). MS obsd. (ESI⁺) [(M + H)⁺]: 473.1. |
| 38 | 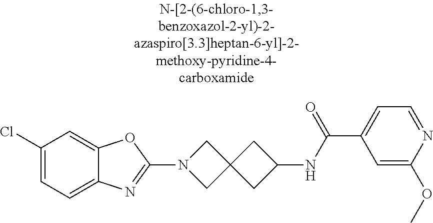 [SMILES: COc1cc(C(=O)NC2CC3(C2)CN(c2nc4ccc(Cl)cc4o2)C3)ccn1] | AMINE: Int- 11 ACID: 2- methoxy- pyridine-4- carboxylic acid | ¹H NMR (400 MHz, DMSO-d₆) δ ppm: 8.85 (d, J = 7.2 Hz, 1H), 8.28 (d, J = 5.2 Hz, 1H), 7.59 (d, J = 2.0 Hz, 1H), 7.33 (dd, J = 5.3, 1.4 Hz, 1H), 7.28 (d, J = 8.4 Hz, 1H), 7.24-7.15 (m, 2H), 4.41-4.25 (m, 3H), 4.19 (s, 2H), 3.89 (s, 3H), 2.67-2.57 (m, 2H), 2.39-2.28 (m, 2H). MS obsd. (ESI⁺) [(M + H)⁺]: 399.1. |
| 39 | 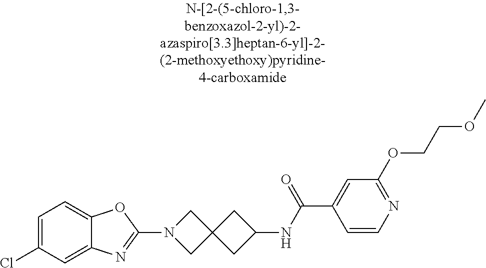 [SMILES: COCCOc1cc(C(=O)NC2CC3(C2)CN(c2nc4cc(Cl)ccc4o2)C3)ccn1] | AMINE: Int-9 ACID: Int-39 | ¹H NMR (400 MHz, DMSO-d₆) δ ppm: 8.83 (d, J = 7.2 Hz, 1H), 8.32-8.20 (m, 1H), 7.43 (d, J = 8.5 Hz, 1H), 7.38-7.28 (m, 2H), 7.21 (s, 1H), 7.05 (dd, J = 8.5, 2.2 Hz, 1H), 4.45-4.37 (m, 2H), 4.37-4.25 (m, 3H), 4.20 (s, 2H), 3.71-3.57 (m, 2H), 3.30 (s, 3H), 2.73-2.56 (m, 2H), 2.41-2.23 (m, 2H). MS obsd. (ESI⁺) [(M + H)⁺]: 443.1. |
| 40 | 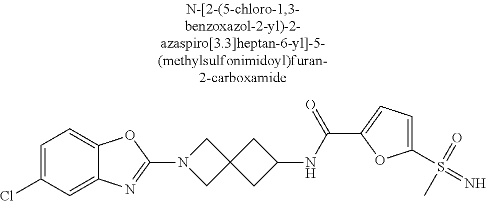 [SMILES: CS(=N)(=O)c1ccc(C(=O)NC2CC3(C2)CN(c2nc4cc(Cl)ccc4o2)C3)o1] | AMINE: Int-9 ACID: Int-33 | ¹H NMR (400 MHz, DMSO-d₆) δ ppm: 8.83 (d, J = 7.5 Hz, 1H), 7.43 (d, J = 8.5 Hz, 1H), 7.34 (d, J = 2.1 Hz, 1H), 7.21 (d, J = 3.6 Hz, 1H), 7.15 (d, J = 3.6 Hz, 1H), 7.05 (dd, J = 8.5, 2.2 Hz, 1H), 4.88 (d, J = 0.9 Hz, 1H), 4.37-4.29 (m, 3H), 4.19 (s, 2H), 3.20 (d, J = 1.1 Hz, 3H), 2.66-2.58 (m, 2H), 2.42-2.34 (m, 2H). MS obsd. (ESI⁺) [(M + H)⁺]: 435.1. |
| 41 | 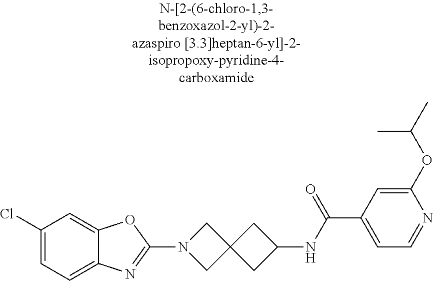 [SMILES: CC(C)Oc1cc(C(=O)NC2CC3(C2)CN(c2nc4ccc(Cl)cc4o2)C3)ccn1] | AMINE: Int- 11 ACID: Int-40 | ¹H NMR (400 MHz, DMSO-d₆) δ ppm: 8.81 (d, J = 7.2 Hz, 1H), 8.25 (dd, J = 5.3, 0.5 Hz, 1H), 7.59 (d, J = 1.9 Hz, 1H), 7.31- 7.24 (m, 2H), 7.20 (dd, J = 8.4, 2.0 Hz, 1H), 7.12 (d, J = 0.6 Hz, 1H), 5.37-5.21 (m, 1H), 4.38- 4.26 (m, 3H), 4.18 (s, 2H), 2.69- 2.56 (m, 2H), 2.40-2.26 (m, 2H), 1.30 (d, J = 6.2 Hz, 6H). MS obsd. (ESI⁺) [(M + H)⁺]: 427.1. |
| 42 | 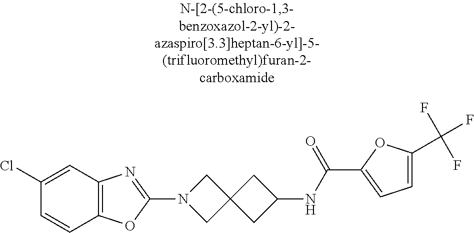 [SMILES: O=C(NC1CC2(C1)CN(c1nc3cc(Cl)ccc3o1)C2)c1ccc(C(F)(F)F)o1] | AMINE: Int- 11 ACID: 5- (trifluoromethyl) furan-2- carboxylic acid | ¹H NMR (400 MHz, DMSO-d₆) δ ppm: 8.88 (d, J = 7.5 Hz, 1H), 7.43 (d, J = 8.5 Hz, 1H), 7.37 (s, 1H), 7.34 (d, J = 2.0 Hz, 1H), 7.27 (d, J = 3.6 Hz, 1H), 7.05 (dd, J = 8.5, 2.0 Hz, 1H), 4.36- 4.26 (m, 3H), 4.19 (s, 2H), 2.62 (d, J = 10.7 Hz, 2H), 2.42-2.32 (m, 2H). MS obsd. (ESI⁺) [(M + H)⁺]: 426.1. |
| 43 | 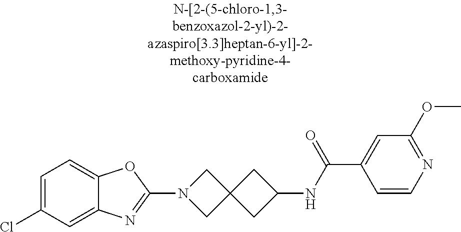 [SMILES: COc1cc(C(=O)NC2CC3(C2)CN(c2nc4cc(Cl)ccc4o2)C3)ccn1] | AMINE: Int-9 ACID: 2- methoxy- pyridine-4- carboxylic acid | ¹H NMR (400 MHz, CDCl₃) δ ppm: 2.82 (t, J = 9.6 Hz, 2H), 2.31 (t, J = 10.3 Hz, 2H). 3.97 (s, 3H), 4.25 (s, 2H), 4.36 (s, 2H), 4.37-4.50 (m, 1H), 6.30 (d, J = 6.3 Hz, 1H), 7.00-7.02 (m, 2H), 7.13-7.17 (m, 2H), 7.33 (s, 1H), 8.26 (d, J = 5.1 Hz, 1H). MS obsd. (ESI⁺) [(M + H)⁺]: 399.1. |
| 44 | 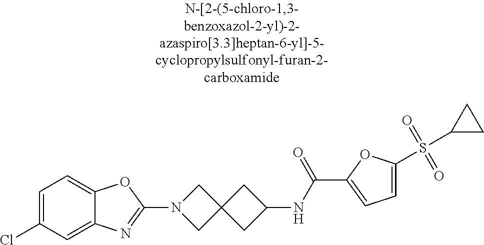 [SMILES: O=C(NC1CC2(C1)CN(c1nc3cc(Cl)ccc3o1)C2)c1ccc(S(=O)(=O)C2CC2)o1] | AMINE: Int-9 ACID: Int-27 | ¹H NMR (400 MHz, DMSO-d₆) δ ppm: 8.90 (d, J = 7.5 Hz, 1H), 7.43 (d, J = 8.5 Hz, 1H), 7.37 (d, J = 3.7 Hz, 1H), 7.34 (d, J = 2.1 Hz, 1H), 7.30 (d, J = 3.7 Hz, 1H), 7.05 (dd, J = 8.5, 2.2 Hz, 1H), 4.39-4.27 (m, 3H), 4.20 (s, 2H), 3.00-2.94 (m, 1H), 2.66-2.58 (m, 2H), 2.43-2.34 (m, 2H), 1.26-1.13 (m, 4H). MS obsd. (ESI⁺) [(M + H)⁺]: 462.1. |
| 45 | 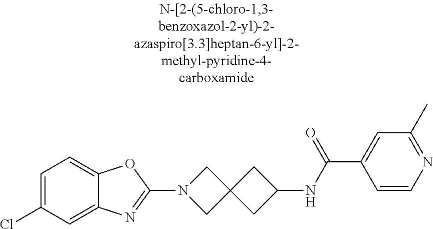 [SMILES: Cc1cc(C(=O)NC2CC3(C2)CN(c2nc4cc(Cl)ccc4o2)C3)ccn1] | AMINE: Int-9 ACID: 2- methylpyridine- 4-carboxylic acid | ¹H NMR (400 MHz, CDCl₃) δ ppm: 8.61 (br d, 1H, J = 4.5 Hz), 7.54 (s, 1H), 7.44 (br d, J = 4.9 Hz, 1H), 7.32 (s, 1H), 7.16 (d, J = 8.4 Hz, 1H), 7.01 (dd, J = 1.3, 8.4 Hz, 1H), 6.45 (br d, J = 6.8 Hz, 1H), 4.4-4.6 (m, 1H), 4.36 (s, 2H), 4.24 (s, 2H), 2.83 (br d, J = 1.6 Hz, 2H), 2.66 (s, 3H), 2.3- 2.4 (m, 2H). MS obsd. (ESI⁺) [(M + H)⁺]: 383.1. |
| 46 | 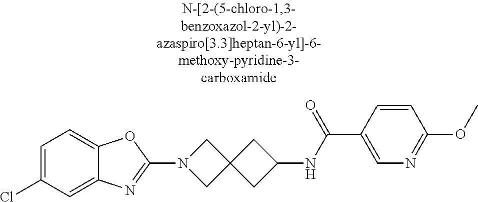 [SMILES: COc1ccc(C(=O)NC2CC3(C2)CN(c2nc4cc(Cl)ccc4o2)C3)cn1] | AMINE: Int-9 ACID: 6- methoxy- pyridine- 3- carboxylic acid | ¹H NMR (400 MHz, CDCl₃) δ ppm: 8.55 (d, J = 1.8 Hz, 1H), 7.97 (dd, J = 2.3, 8.6 Hz, 1H), 7.33 (d, J = 1.8 Hz, 1H), 7.17 (d, J = 8.4 Hz, 1H), 7.02 (dd, J = 2.0, 8.6 Hz, 1H), 6.78 (d, J = 8.6 Hz, 1H), 6.19 (br d, J = 6.7 Hz, 1H), 4.51 (qd, J = 8.0, 15.7 Hz, 1H), 4.38 (s, 2H), 4.28 (s, 2H), 3.98 (s, 3H), 2.82 (br t, J = 10.0 Hz, 2H), 2.38-2.25 (m, 2H). MS obsd. (ESI⁺) [(M + H)⁺]: 399.1. |
| 49 | 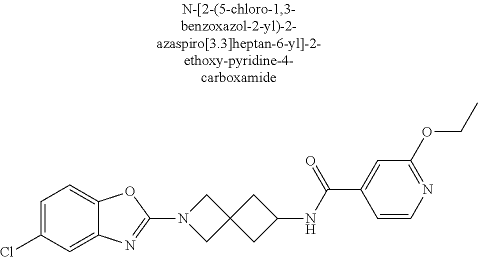 [SMILES: CCOc1cc(C(=O)NC2CC3(C2)CN(c2nc4cc(Cl)ccc4o2)C3)ccn1] | AMINE: Int-9 ACID: 2- ethoxypyridine- 4-carboxylic acid | ¹H NMR (400 MHz, DMSO-d₆) δ ppm: 8.82 (d, J = 7.2 Hz, 1H), 8.29-8.22 (m, 1H), 7.43 (d, J = 8.5 Hz, 1H), 7.34 (d, J = 2.1 Hz, 1H), 7.31 (dd, J = 5.3, 1.4 Hz, 1H), 7.16 (s, 1H), 7.05 (dd, J = 8.5, 2.2 Hz, 1H), 4.41-4.24 (m, 5H), 4.19 (s, 2H), 2.69-2.58 (m, 2H), 2.39-2.29 (m, 2H), 1.32 (t, J = 7.0 Hz, 3H). MS obsd. (ESI⁺) [(M + H)⁺]: 413.1. |
| 50 | 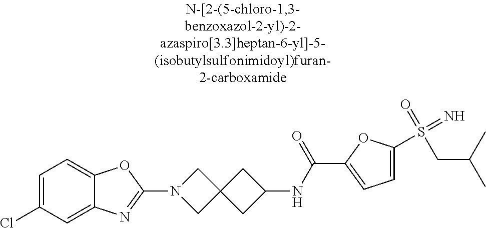 [SMILES: CC(C)CS(=N)(=O)c1ccc(C(=O)NC2CC3(C2)CN(c2nc4cc(Cl)ccc4o2)C3)o1] | AMINE: Int-9 ACID: Int-36 | ¹H NMR (400 MHz, DMSO-d₆) δ ppm: 8.81 (d, J = 7.6 Hz, 1H), 7.43 (d, J = 8.5 Hz, 1H), 7.34 (d, J = 2.1 Hz, 1H), 7.22 (d, J = 3.6 Hz, 1H), 7.16 (d, J = 3.6 Hz, 1H), 7.05 (dd, J = 8.5, 2.2 Hz, 1H), 4.88 (s, 1H), 4.38-4.28 (m, 3H), 4.19 (s, 2H), 3.18 (qd, J = 14.1, 6.4 Hz, 2H), 2.67-2.57 (m, 2H), 2.42-2.34 (m, 2H), 2.15 (dt, J = 13.2, 6.6 Hz, 1H), 0.96 (dd, J = 6.7, 2.9 Hz, 6H). MS obsd. (ESI⁺) [(M + H)⁺]: 477.2. |
| 51 | 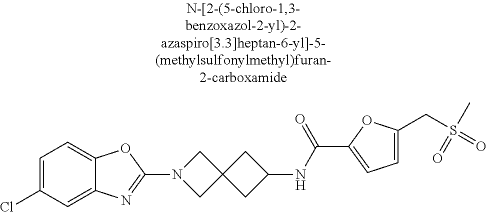 [SMILES: CS(=O)(=O)Cc1ccc(C(=O)NC2CC3(C2)CN(c2nc4cc(Cl)ccc4o2)C3)o1] | AMINE: Int-9 ACID: Int-32 | ¹H NMR (400 MHz, DMSO-d₆) δ ppm: 8.52 (d, J = 7.6 Hz, 1H), 7.43 (d, J = 8.5 Hz, 1H), 7.34 (d, J = 2.1 Hz, 1H), 7.14 (d, J = 3.4 Hz, 1H), 7.05 (dd, J = 8.5, 2.1 Hz, 1H), 6.66 (d, J = 3.4 Hz, 1H), 4.70 (s, 2H), 4.36-4.25 (m, 3H), 4.18 (s, 2H), 3.04 (s, 3H), 2.64-2.55 (m, 2H), 2.39- 2.30 (m, 2H). MS obsd. (ESI⁺) [(M + H)⁺]: 450.1. |
| 52 | 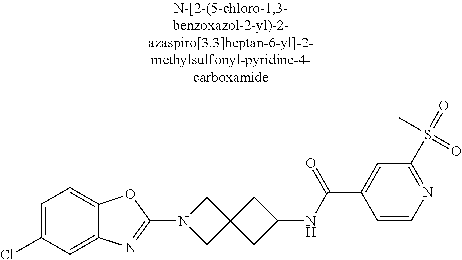 | AMINE: Int-9 ACID: Int-41 | ¹H NMR (400 MHz, CDCl₃) δ ppm: 8.86 (dd, J = 4.9, 0.7 Hz, 1H), 8.33 (d, J = 0.7 Hz, 1H), 8.02 (dd, J = 4.9, 1.6 Hz, 1H), 7.33 (d, J = 2.0 Hz, 1H), 7.17 (d, J = 8.5 Hz, 1H), 7.02 (dd, J = 8.5, 2.1 Hz, 1H), 6.92 (d, J = 7.2 Hz, 1H), 4.54 (dd, J = 15.9, 8.1 Hz, 1H), 4.39 (s, 2H), 4.29 (s, 2H), 3.28 (s, 3H), 2.88-2.78 (m, 2H), 2.44-2.34 (m, 2H). MS obsd. (ESI⁺) [(M + H)⁺]: 477.1. |
| 53 | 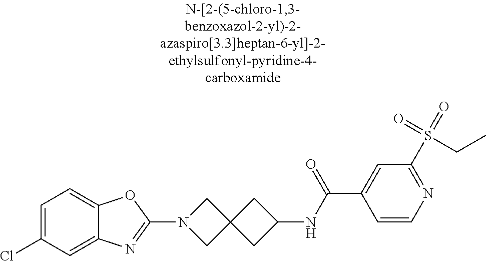 [SMILES: CCS(=O)(=O)c1cc(C(=O)NC2CC3(C2)CN(c2nc4cc(Cl)ccc4o2)C3)ccn1] | AMINE: Int-9 ACID: Int-42 | ¹H NMR (400 MHz, DMSO-d₆) δ ppm: 9.26 (d, J = 7.1 Hz, 1H), 8.96 (d, J = 4.9 Hz, 1H), 8.44 (s, 1H), 8.11 (dd, J = 4.9, 1.4 Hz, 1H), 7.43 (d, J = 8.5 Hz, 1H), 7.35 (d, J = 2.1 Hz, 1H), 7.05 (dd, J = 8.5, 2.1 Hz, 1H), 4.47- 4.28 (m, 3H), 4.21 (s, 2H), 3.49 (q, J = 7.4 Hz, 2H), 2.76-2.60 (m, 2H), 2.44-2.30 (m, 2H), 1.14 (t, J = 7.4 Hz, 3H). MS obsd. (ESI⁺) [(M + H)⁺]: 461.1. |
| 54 | 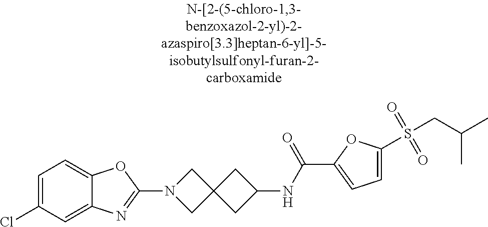 [SMILES: CC(C)CS(=O)(=O)c1ccc(C(=O)NC2CC3(C2)CN(c2nc4cc(Cl)ccc4o2)C3)o1] | AMINE: Int-9 ACID: Int-31 | ¹H NMR (400 MHz, CDCl₃) δ ppm: 7.33 (d, J = 2.0 Hz, 1H), 7.24-7.19 (m, 2H), 7.16 (d, J = 8.5 Hz, 1H), 7.01 (dd, J = 8.5, 2.0 Hz, 1H), 6.77 (d, J = 7.6 Hz, 1H), 4.49 (dt, J = 16.5, 8.3 Hz, 1H), 4.31 (d, J = 38.6 Hz, 4H), 3.13 (d, J = 6.5 Hz, 2H), 2.86- 2.73 (m, 2H), 2.44-2.24 (m, 3H), 1.11 (d, J = 6.7 Hz, 6H). MS obsd. (ESI⁺) [(M + H)⁺]: 478.1. |
| 55 | 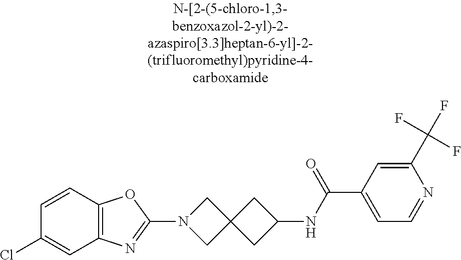 [SMILES: O=C(NC1CC2(C1)CN(c1nc3cc(Cl)ccc3o1)C2)c1ccnc(C(F)(F)F)c1] | AMINE: Int-9 ACID: 2- (trifluoromethyl) pyridine-4- carboxylic acid | ¹H NMR (400 MHz, CDCl₃) δ ppm: 8.88 (d, J = 4.9 Hz, 1H), 7.98 (s, 1H), 7.81 (d, J = 4.9 Hz, 1H), 7.33 (d, J = 2.0 Hz, 1H), 7.16 (d, J = 8.5 Hz, 1H), 7.01 (dd, J = 8.5, 2.1 Hz, 1H), 6.42 (d, J = 6.8 Hz, 1H), 4.54 (dq, J = 16.0, 7.9 Hz, 1H), 4.38 (s, 2H), 4.27 (s, 2H), 2.90-2.80 (m, 2H), 2.41-2.32 (m, 2H). MS obsd. (ESI⁺) [(M + H)⁺]: 437.1. |
| 56 | 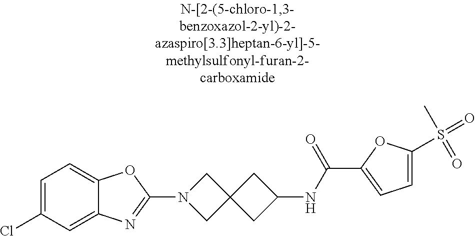 [SMILES: CS(=O)(=O)c1ccc(C(=O)NC2CC3(C2)CN(c2nc4cc(Cl)ccc4o2)C3)o1] | AMINE: Int-9 ACID: Int-24 | ¹H NMR (400 MHz, CDCl₃) δ ppm: 7.33 (d, J = 2.0 Hz, 1H), 7.22 (dd, J = 9.1, 3.7 Hz, 2H), 7.16 (d, J = 8.5 Hz, 1H), 7.00 (dd, J = 8.5, 2.1 Hz, 1H), 6.73 (d, J = 7.6 Hz, 1H), 4.51 (dd, J = 16.4, 8.5 Hz, 1H), 4.35 (s, 2H), 4.26 (s, 2H), 3.21 (s, 3H), 2.85- 2.75 (m, 2H), 2.45-2.30 (m, 2H). MS obsd. (ESI⁺) [(M + H)⁺]: 436.1. |
| 57 | 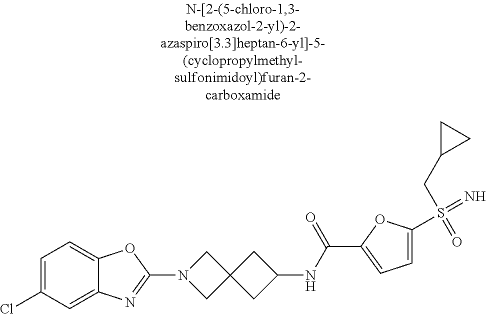 [SMILES: N=S(=O)(CC1CC1)c1ccc(C(=O)NC2CC3(C2)CN(c2nc4cc(Cl)ccc4o2)C3)o1] | AMINE: Int-9 ACID: Int-35 | ¹H NMR (400 MHz, CD₃OD) δ ppm: 7.30-7.22 (m, 4H), 7.04 (dd, J = 8.5, 2.1 Hz, 1H), 4.44 (dd, J = 16.4, 8.2 Hz, 1H), 4.38 (s, 2H), 4.25 (s, 2H), 3.33 (s, 2H), 2.80-2.69 (m, 2H), 2.47- 2.37 (m, 2H), 1.10 (qd, J = 7.4, 3.7 Hz, 1H), 0.63-0.46 (m, 2H), 0.21-0.08 (m, 2H). MS obsd. (ESI⁺) [(M + H)⁺]: 475.1. |
| 58 | 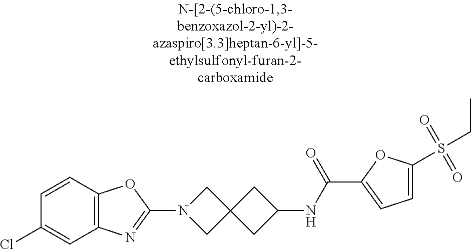 [SMILES: CCS(=O)(=O)c1ccc(C(=O)NC2CC3(C2)CN(c2nc4cc(Cl)ccc4o2)C3)o1] | AMINE: Int-9 ACID: Int-26 | ¹H NMR (400 MHz, DMSO-d₆) δ ppm: 8.90 (d, J = 7.3 Hz, 1H), 7.41 (d, J = 3.6 Hz, 2H), 7.34 (d, J = 2.1 Hz, 1H), 7.29 (s, 1H), 7.05 (dd, J = 8.5, 2.2 Hz, 1H), 4.31 (m, 3H), 4.19 (s, 2H), 3.46 (d, J = 7.4 Hz, 2H), 2.63 (d, J = 10.6 Hz, 2H), 2.40-2.33 (m, 2H), 1.17 (t, J = 7.4 Hz, 3H). MS obsd. (ESI⁺) [(M + H)⁺]: 450.1. |
| 59 | 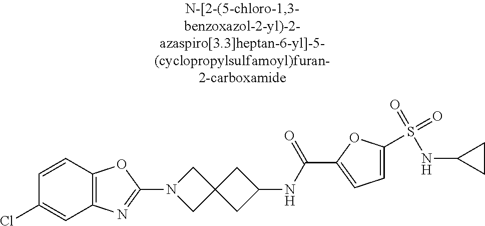 [SMILES: O=C(NC1CC2(C1)CN(c1nc3cc(Cl)ccc3o1)C2)c1ccc(S(=O)(=O)NC2CC2)o1] | AMINE: Int-9 ACID: Int-37 | ¹H NMR (400 MHz, CD₃OD) δ ppm: 7.29 (d, J = 8.6 Hz, 1H), 7.26 (d, J = 2.1 Hz, 1H), 7.23 (d, J = 3.5 Hz, 1H), 7.17 (d, J = 3.7 Hz, 1H), 7.06 (dd, J = 2.1, 8.6 Hz, 1H), 4.49-4.41 (m, 1H), 4.39 (s, 2H), 4.26 (s, 2H), 2.80- 2.70 (m, 2H), 2.48-2.36 (m, 3H), 2.05 (s, 1H), 0.63 (dd, J = 2.1, 7.1 Hz, 2H), 0.56-0.50 (m, 2H). MS obsd. (ESI⁺) [(M + H)⁺]: 477.1. |
| 60 | 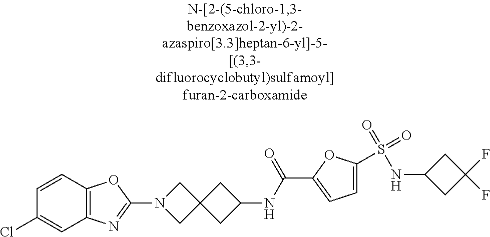 [SMILES: O=C(NC1CC2(C1)CN(c1nc3cc(Cl)ccc3o1)C2)c1ccc(S(=O)(=O)NC2CC(F)(F)C2)o1] | AMINE: Int-9 ACID: Int-38 | ¹H NMR (400 MHz, CD₃OD) δ ppm: 7.21 (d, J = 8.6 Hz, 1H), 7.18 (d, J = 2.0 Hz, 1H), 7.13 (d, J = 3.7 Hz, 1H), 7.06 (d, J = 3.5 Hz, 1H), 6.97 (dd, J = 2.1, 8.5 Hz, 1H), 4.41-4.33 (m, 1H), 4.31 (s, 2H), 4.18 (s, 2H), 3.77 (ddt, J = 2.0, 5.9, 7.6 Hz, 1H), 2.84-2.72 (m, 2H), 2.71-2.63 (m, 2H), 2.48-2.30 (m, 4H). MS obsd. (ESI⁺) [(M + H)⁺]: 527.1. |
| 61 | 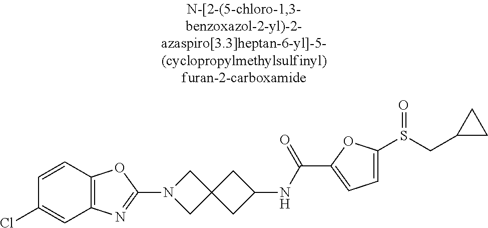 [SMILES: O=C(NC1CC2(C1)CN(c1nc3cc(Cl)ccc3o1)C2)c1ccc(S(=O)CC2CC2)o1] | AMINE: Int-9 ACID: Int-28 | ¹H NMR (400 MHz, CD₃OD) δ ppm: 7.28 (d, J = 8.5 Hz, 1H), 7.23 (dt, J = 5.3, 2.8 Hz, 3H), 7.04 (dd, J = 8.5, 2.1 Hz, 1H), 4.49-4.39 (m, 1H), 4.37 (s, 2H), 4.25 (s, 2H), 3.38 (dd, J = 13.2, 7.1 Hz, 1H), 3.28-3.19 (m, 1H), 2.80-2.65 (m, 2H), 2.50-2.34 (m, 2H), 0.95 (qd, J = 7.7, 3.9 Hz, 1H), 0.72-0.57 (m, 2H), 0.49-0.37 (m, 1H), 0.30 (dq, J = 9.8, 4.8 Hz, 1H). MS obsd. (ESI⁺) [(M + H)⁺]: 460.1. |
| 62 | 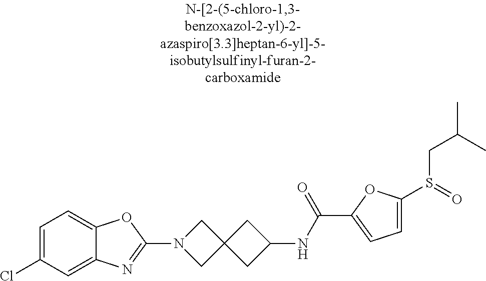 [SMILES: CC(C)CS(=O)c1ccc(C(=O)NC2CC3(C2)CN(c2nc4cc(Cl)ccc4o2)C3)o1] | AMINE: Int-9 ACID: Int-30 | ¹H NMR (400 MHz, DMSO-d₆) δ ppm: 8.84 (d, J = 7.6 Hz, 1H), 7.43 (d, J = 8.5 Hz, 1H), 7.34 (d, J = 2.1 Hz, 1H), 7.30 (d, J = 3.6 Hz, 1H), 7.23 (d, J = 3.6 Hz, 1H), 7.05 (dd, J = 8.5, 2.2 Hz, 1H), 4.40-4.26 (m, 3H), 4.19 (s, 2H), 3.36 (dd, J = 12.9, 5.6 Hz, 1H), 2.98 (dd, J = 12.8, 8.5 Hz, 1H), 2.63 (ddd, J = 12.1, 10.3, 3.0 Hz, 2H), 2.38 (ddd, J = 17.0, 10.4, 6.1 Hz, 2H), 2.07- 1.89 (m, 1H), 1.05 (dd, J = 6.7, 3.1 Hz, 6H). MS obsd. (ESI⁺) [(M + H)⁺]: 462.1. |
| 64 | 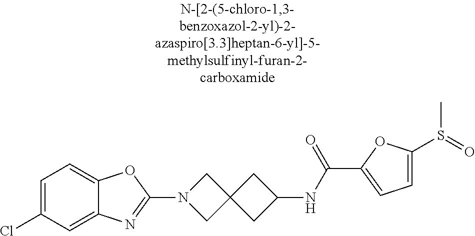 [SMILES: CS(=O)c1ccc(C(=O)NC2CC3(C2)CN(c2nc4cc(Cl)ccc4o2)C3)o1] | AMINE: Int-9 ACID: Int-23 | ¹H NMR (400 MHz, DMSO-d₆) δ ppm: 8.85 (d, J = 7.6 Hz, 1H), 7.43 (d, J = 8.5 Hz, 1H), 7.34 (d, J = 2.1 Hz, 1H), 7.27 (d, J = 3.6 Hz, 1H), 7.23 (d, J = 3.6 Hz, 1H), 7.05 (dd, J = 8.5, 2.1 Hz, 1H), 4.39-4.29 (m, 3H), 4.19 (s, 2H), 3.04 (s, 3H), 2.65-2.58 (m, 2H), 2.42-2.35 (m, 2H). MS obsd. (ESI⁺) [(M + H)⁺]: 420.1. |
| 65 | 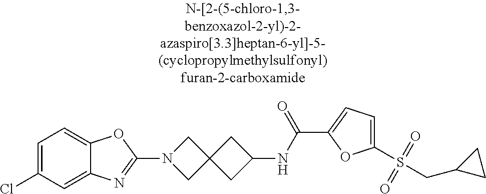 [SMILES: O=C(NC1CC2(C1)CN(c1nc3cc(Cl)ccc3o1)C2)c1ccc(S(=O)(=O)CC2CC2)o1] | AMINE: Int-9 ACID: Int-29 | ¹H NMR (400 MHz, DMSO-d₆) δ ppm: 8.88 (d, J = 7.5 Hz, 1H), 7.42 (dd, J = 9.8, 6.1 Hz, 2H), 7.34 (d, J = 2.1 Hz, 1H), 7.30 (d, J = 3.7 Hz, 1H), 7.05 (dd, J = 8.5, 2.2 Hz, 1H), 4.39-4.26 (m, 3H), 4.19 (s, 2H), 3.43 (d, J = 7.2 Hz, 2H), 2.68-2.58 (m, 2H), 2.42-2.31 (m, 2H), 0.99-0.85 (m, 1H), 0.58-0.39 (m, 2H), 0.22-0.06 (m, 2H). MS obsd. (ESI⁺) [(M + H)⁺]: 476.1. |
| 66 | 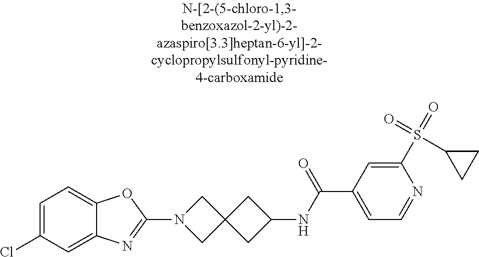 [SMILES: O=C(NC1CC2(C1)CN(c1nc3cc(Cl)ccc3o1)C2)c1ccnc(S(=O)(=O)C2CC2)c1] | AMINE: Int-9 ACID: Int-43 | ¹H NMR (400 MHz, DMSO-d₆) δ ppm: 9.17 (d, J = 6.9 Hz, 1H), 8.90 (d, J = 4.8 Hz, 1H), 8.31 (s, 1H), 8.03 (d, J = 4.4 Hz, 1H), 7.36 (d, J = 8.4 Hz, 1H), 7.27 (s, 1H), 6.98 (d, J = 8.4 Hz, 1H), 4.35-4.22 (m, 3H), 4.14 (s, 2H), 2.93 (dd, J = 11.1, 6.2 Hz, 1H), 2.63-2.55 (m, 2H), 2.37- 2.23 (m, 2H), 1.06 (dd, J = 9.8, 7.1 Hz, 4H). MS obsd. (ESI⁺) [(M + H)⁺]: 473.1. |
| 67 | 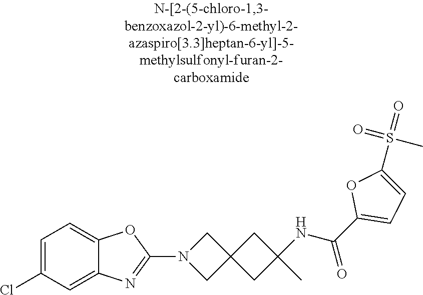 [SMILES: CC1(NC(=O)c2ccc(S(C)(=O)=O)o2)CC2(CN(c3nc4cc(Cl)ccc4o3)C2)C1] | AMINE: Int- 12 ACID: Int-24 | ¹H NMR (400 MHz, DMSO-d₆) δ ppm: 9.00 (s, 1H), 8.92 (d, J = 5.0 Hz, 1H), 8.24 (s, 1H), 8.07 (d, J = 4.9 Hz, 1H), 7.42 (d, J = 8.4 Hz, 1H), 7.33 (d, J = 2.1 Hz, 1H), 7.05 (dd, J = 8.4, 2.1 Hz, 1H), 4.32 (s, 2H), 4.22 (s, 2H), 2.66 (br d, J = 13.2 Hz, 2H), 2.39-2.45 (m, 2H), 1.43 (s, 3H). MS obsd. (ESI⁺) [(M + H)⁺]: 450.1. |
| 68 | 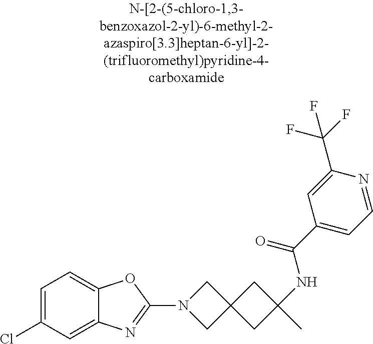 [SMILES: CC1(NC(=O)c2ccnc(C(F)(F)F)c2)CC2(CN(c3nc4cc(Cl)ccc4o3)C2)C1] | AMINE: Int- 12 ACID: 2- (trifluoromethyl) pyridine-4- carboxylic acid | ¹H NMR (400 MHz, DMSO-d₆) δ ppm: 8.75 (s, 1H), 7.32-7.45 (m, 3H), 7.25-7.30 (m, 1H), 7.04 (d, J = 8.6 Hz, 1H), 4.27- 4.35 (m, 2H), 4.16-4.23 (m, 2H), 3.38-3.41 (m, 3H), 2.63 (br d, J = 13.3 Hz, 2H), 2.41 (br d, J = 13.6 Hz, 2H), 1.41 (s, 3H). MS obsd. (ESI⁺) [(M + H)⁺]: 451.1. |
| 69 | 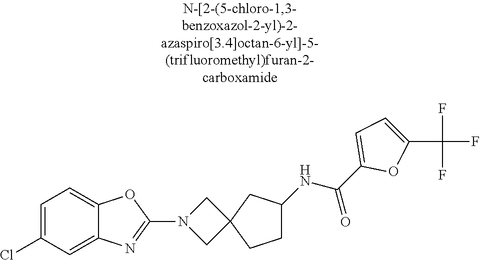 [SMILES: O=C(NC1CCC2(C1)CN(c1nc3cc(Cl)ccc3o1)C2)c1ccc(C(F)(F)F)o1] | AMINE: Int- 13 ACID: 5- (trifluoromethyl) furan-2- carboxylic acid | ¹H NMR (400 MHz, DMSO-d₆) δ ppm: 8.65 (d, J = 7.4 Hz, 1H), 7.43 (d, J = 8.5 Hz, 1H), 7.39- 7.32 (m, 2H), 7.31-7.25 (m, 1H), 7.05 (dd, J = 8.5, 2.2 Hz, 1H), 4.32-4.21 (m, 1H), 4.18 (d, J = 8.1 Hz, 1H), 4.15-4.09 (m, 2H), 4.05 (d, J = 8.0 Hz, 1H), 2.32 (dd, J = 13.4, 8.0 Hz, 1H), 2.10-1.87 (m, 4H), 1.68- 1.59 (m, 1H). MS obsd. (ESI⁺) [(M + H)⁺]: 440.1. |
| 70 | 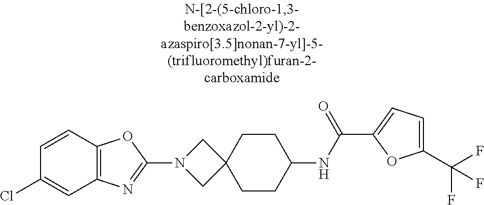 [SMILES: O=C(NC1CCC2(CC1)CN(c1nc3cc(Cl)ccc3o1)C2)c1ccc(C(F)(F)F)o1] | AMINE: Int- 14 ACID: 5- (trifluoromethyl) furan-2- carboxylic acid | ¹H NMR (400 MHz, CD₃OD) δ ppm: 7.32 (d, J = 8.4 Hz, 1H), 7.28 (d, J = 2.1 Hz, 1H), 7.22 (d, J = 3.7 Hz, 1H), 7.16-7.13 (m, 1H), 7.08 (dd, J = 2.1, 8.6 Hz, 1H), 4.08 (s, 2H), 3.99 (s, 2H), 3.95-3.86 (m, 1H), 2.14 (br d, J = 13.4 Hz, 2H), 1.99-1.90 (m, 2H), 1.74 (dt, J = 3.3, 13.3 Hz, 2H), 1.59-1.46 (m, 2H). MS obsd. (ESI⁺) [(M + H)⁺]: 454.1 |
| 71 | 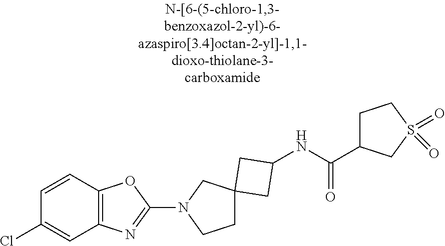 [SMILES: O=C(NC1CC2(CCN(c3nc4cc(Cl)ccc4o3)C2)C1)C1CCS(=O)(=O)C1] | AMINE: Int- 16 ACID: 1,1- dioxothiolane- 3-carboxylic acid | ¹H NMR (400 MHz, CD₃OD) δ ppm: 7.50 (t, J = 9.0 Hz, 1H), 7.40 (dd, J = 1.9, 7.9 Hz, 1H), 7.28 (dt, J = 2.1, 8.8 Hz, 1H), 4.36 (t, J = 8.1 Hz, 1H), 3.86- 3.74 (m, 3H), 3.71 (s, 1H), 3.29- 3.20 (m, 4H), 3.18-3.06 (m, 1H), 2.57-2.41 (m, 3H), 2.31- 2.17 (m, 4H), 2.17-2.14 (m, 1H), 2.05 (br d, J = 5.7 Hz, 1H). MS obsd. (ESI⁺) [(M + H)⁺]: 424.1. |
| 72 | 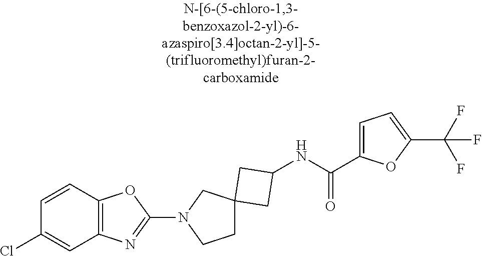 [SMILES: O=C(NC1CC2(CCN(c3nc4cc(Cl)ccc4o3)C2)C1)c1ccc(C(F)(F)F)o1] | AMINE: Int- 16 ACID: 5- (trifluoromethyl) furan-2- carboxylic acid | ¹H NMR (400 MHz, CD₃OD) δ ppm: 7.76 (t, J = 2.6 Hz, 1H), 7.22 (d, J = 3.3 Hz, 1H), 7.14 (d, J = 2.8 Hz, 1H), 6.92-6.84 (m, 1H), 6.83-6.77 (m, 1H), 4.56 (dt, J = 2.9, 8.2 Hz, 1H), 3.60- 3.45 (m, 4H), 2.52-2.40 (m, 2H), 2.31-2.22 (m, 2H), 2.14- 2.01 (m, 2H). MS obsd. (ESI⁺) [(M + H)⁺]: 440.1. |
| 73 | 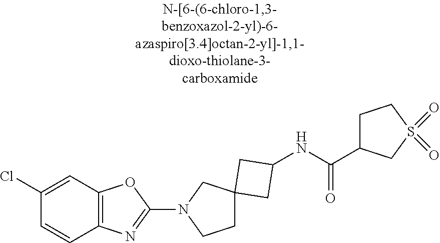 [SMILES: O=C(NC1CC2(CCN(c3nc4ccc(Cl)cc4o3)C2)C1)C1CCS(=O)(=O)C1] | AMINE: Int- 15 ACID: 1,1- dioxothiolane- 3-carboxylic acid | ¹H NMR (400 MHz, CD₃OD) & ppm: 7.40 (dd, J = 1.7, 4.0 Hz, 1H), 7.26-7.16 (m, 2H), 4.36 (dq, J = 2.6, 7.8 Hz, 1H), 3.74- 3.58 (m, 4H), 3.30-3.19 (m, 4H), 3.09 (td, J = 8.6, 13.2 Hz, 1H), 2.52-2.40 (m, 3H), 2.33- 2.21 (m, 1H), 2.19-2.06 (m, 4H). MS obsd. (ESI⁺) [(M + H)⁺]: 424.1. |
| 74 | 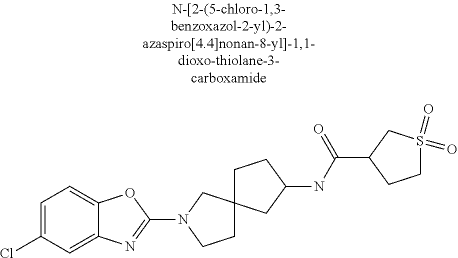 [SMILES: O=C(NC1CCC2(CCN(c3nc4cc(Cl)ccc4o3)C2)C1)C1CCS(=O)(=O)C1] | AMINE: Int- 17 ACID: 1,1- dioxothiolane- 3-carboxylic acid | ¹H NMR (400 MHz, DMSO-d₆) δ ppm: 8.20 (d, J = 6.7 Hz, 1H), 7.40 (d, J = 8.4 Hz, 1H), 7.29 (d, J = 2.1 Hz, 1H), 7.04-6.95 (m, 1H), 4.19-4.06 (m, 1H), 3.64- 3.52 (m, 4H), 3.31-3.01 (m, 5H), 2.34-2.26 (m, 1H), 2.13- 1.86 (m, 5H), 1.79-1.43 (m, 4H). MS obsd. (ESI⁺) [(M + H)⁺]: 438.1. |
| 75 | 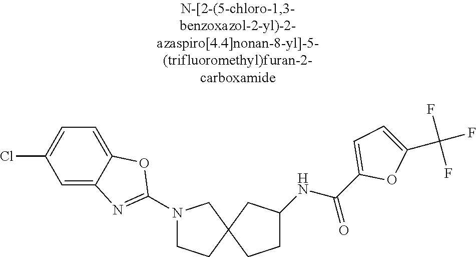 [SMILES: O=C(NC1CCC2(CCN(c3nc4cc(Cl)ccc4o3)C2)C1)c1ccc(C(F)(F)F)o1] | AMINE: Int- 17 ACID: 5- (trifluoromethyl) furan-2- carboxylic | ¹H NMR (400 MHz, DMSO-d₆) δ ppm: 8.66 (dd, J = 7.4, 3.5 Hz, 1H), 7.40 (d, J = 8.4 Hz, 1H), 7.38-7.35 (m, 1H), 7.29 (dd, J = 3.9, 2.7 Hz, 2H), 7.00 (dd, J = 8.4, 2.2 Hz, 1H), 4.40-4.31 (m, 1H), 3.68-3.58 (m, 2H), 3.57- 3.50 (m, 1H), 3.45 (s, 1H), 2.10- 1.90 (m, 4H), 1.83-1.59 (m, 4H). MS obsd. (ESI⁺) [(M + H)⁺]: 454.1. |
| 76 | 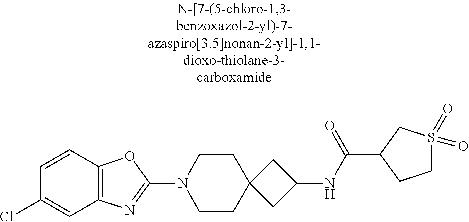 [SMILES: O=C(NC1CC2(CCN(c3nc4cc(Cl)ccc4o3)CC2)C1)C1CCS(=O)(=O)C1] | AMINE: Int- 18 ACID: 1,1- dioxothiolane- 3-carboxylic acid | ¹H NMR (400 MHz, CD₃OD) δ ppm: 7.36 (d, J = 8.6 Hz, 1H), 7.29 (d, J = 2.0 Hz, 1H), 7.12 (dd, J = 2.1, 8.6 Hz, 1H), 4.40- 4.30 (m, 1H), 3.74-3.70 (m, 2H), 3.67-3.62 (m, 2H), 3.31- 3.20 (m, 4H), 3.09 (ddd, J = 7.8, 9.3, 13.2 Hz, 1H), 2.50-2.35 (m, 3H), 2.31-2.20 (m, 1H), 1.89-1.80 (m, 4H), 1.79-1.74 (m, 2H). MS obsd. (ESI⁺) [(M + H)⁺]: 438.1. |
| 77 | 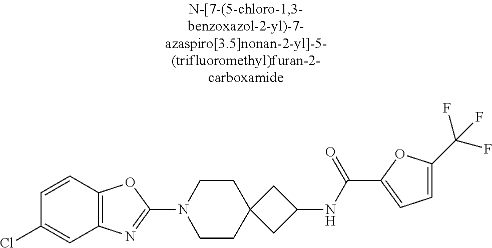 [SMILES: O=C(NC1CC2(CCN(c3nc4cc(Cl)ccc4o3)CC2)C1)c1ccc(C(F)(F)F)o1] | AMINE: Int- 18 ACID: 5- (trifluoromethyl) furan-2- carboxylic acid | ¹H NMR (400 MHz, CD₃OD) δ ppm: 7.33 (d, J = 8.4 Hz, 1H), 7.27 (d, J = 2.1 Hz, 1H), 7.22 (dd, J = 0.7, 3.6 Hz, 1H), 7.16- 7.13 (m, 1H), 7.08 (dd, J = 2.1, 8.4 Hz, 1H), 4.57 (quin, J = 8.4 Hz, 1H), 3.76-3.71 (m, 2H), 3.68-3.62 (m, 2H), 2.45-2.37 (m, 2H), 2.08-2.00 (m, 2H), 1.89-1.83 (m, 2H), 1.79 (dd, J = 4.8, 6.5 Hz, 2H). MS obsd. (ESI⁺) [(M + H)⁺]: 454.1. |
| 78 | 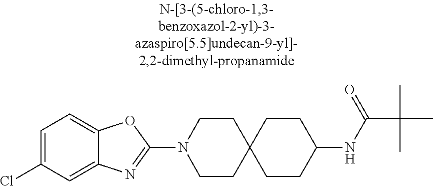 [SMILES: CC(C)(C)C(=O)NC1CCC2(CC1)CCN(c1nc3cc(Cl)ccc3o1)CC2] | AMINE: Int- 19 ACID: 2,2- dimethyl- propanoic acid | ¹H NMR (400 MHz, DMSO-d₆) δ ppm: 7.32 (d, J = 8.4 Hz, 1H), 7.23 (d, J = 2.0 Hz, 1H), 7.03 (d, J = 8.2 Hz, 1H), 6.94 (dd, J = 8.4, 2.1 Hz, 1H), 3.57-3.49 (m, 4H), 1.65 (d, J = 12.9 Hz, 2H), 1.59-1.50 (m, 2H), 1.38 (dt, J = 11.0, 9.2 Hz, 6H), 1.21-1.06 (m, 3H), 1.01 (s, 9H). MS obsd. (ESI⁺) [(M + H)⁺]: 404.0. |
| 79 | 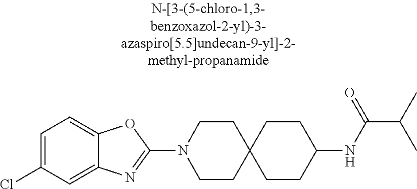 [SMILES: CC(C)C(=O)NC1CCC2(CC1)CCN(c1nc3cc(Cl)ccc3o1)CC2] | AMINE: Int- 19 ACID: 2- methyl- propanoic acid | ¹H NMR (400 MHz, DMSO-d₆) δ ppm: 7.55 (d, J = 7.8 Hz, 1H), 7.39 (d, J = 8.4 Hz, 1H), 7.30 (d, J = 2.2 Hz, 1H), 7.01 (dd, J = 2.2, 8.4 Hz, 1H), 3.64-3.57 (m, 4H), 3.56-3.45 (m, 1H), 2.31 (quin, J = 6.8 Hz, 1H), 1.70 (br d, J = 13.2 Hz, 2H), 1.61-1.53 (m, 4H), 1.46-1.39 (m, 2H), 1.39-1.27 (m, 2H), 1.23-1.14 (m, 2H), 0.97 (d, J = 6.8 Hz, 6H). MS obsd. (ESI⁺) [(M + H)⁺]: 390.2. |
| 80 | 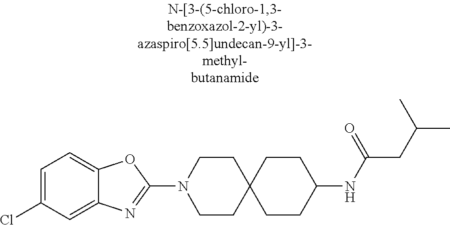 [SMILES: CC(C)CC(=O)NC1CCC2(CC1)CCN(c1nc3cc(Cl)ccc3o1)CC2] | AMINE: Int- 19 ACID: 3- methylbutanoic acid | ¹H NMR (400 MHz, DMSO-d₆) δ ppm: 7.57 (d, J = 7.6 Hz, 1H), 7.32 (d, J = 8.4 Hz, 1H), 7.23 (s, 1H), 6.94 (d, J = 8.4 Hz, 1H), 3.53 (s, 5H), 1.85 (s, 2H), 1.63 (d, J = 13.0 Hz, 2H), 1.50 (s, 4H), 1.35 (s, 2H), 1.30-1.07 (m, 5H), 0.80 (d, J = 4.7 Hz, 6H). MS obsd. (ESI⁺) [(M + H)⁺]: 404.1. |
| 81 | 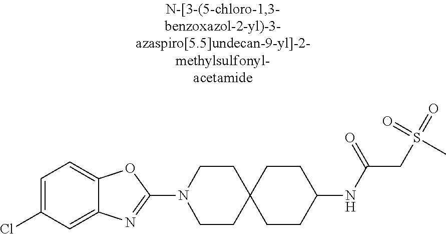 [SMILES: CS(=O)(=O)CC(=O)NC1CCC2(CC1)CCN(c1nc3cc(Cl)ccc3o1)CC2] | AMINE: Int- 19 ACID: 2- methylsulfonyl acetic acid | ¹H NMR (400 MHz, DMSO-d₆) δ ppm: 8.25 (d, J = 7.5 Hz, 1H), 7.39 (d, J = 8.4 Hz, 1H), 7.30 (d, J = 1.9 Hz, 1H), 7.01 (dd, J = 8.4, 2.0 Hz, 1H), 4.03 (s, 2H), 3.60 (s, 5H), 3.10 (s, 3H), 1.76- 1.52 (m, 6H), 1.32 (ddd, J = 41.5, 25.7, 14.9 Hz, 6H). MS obsd. (ESI⁺) [(M + H)⁺]: 439.9. |
| 82 | 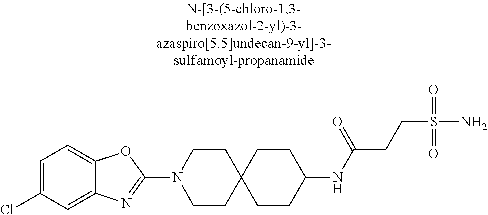 [SMILES: NS(=O)(=O)CCC(=O)NC1CCC2(CC1)CCN(c1nc3cc(Cl)ccc3o1)CC2] | AMINE: Int- 19 ACID: 3- sulfamoyl- propanoic acid | ¹H NMR (400 MHz, DMSO) δ ppm: 7.92 (d, J = 5.6 Hz, 1H), 7.53-7.24 (m, 2H), 7.10-6.69 (m, 3H), 3.60 (s, 6H), 3.18 (s, 2H), 1.78-1.53 (m, 6H), 1.48- 1.03 (m, 7H). MS obsd. (ESI⁺) [(M + H)⁺]: 455.1. |
| 83 | 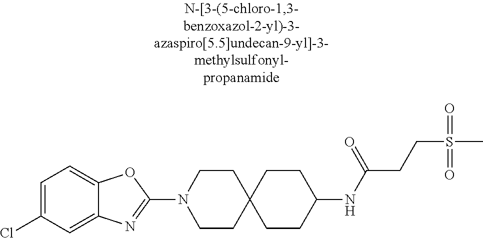 [SMILES: CS(=O)(=O)CCC(=O)NC1CCC2(CC1)CCN(c1nc3cc(Cl)ccc3o1)CC2] | AMINE: Int- 19 ACID: 3- methylsulfonyl propanoic acid | ¹H NMR (400 MHz, DMSO-d₆) δ ppm: 7.90 (d, J = 7.7 Hz, 1H), 7.32 (d, J = 8.4 Hz, 1H), 7.23 (d, J = 2.1 Hz, 1H), 6.94 (dd, J = 8.4, 2.1 Hz, 1H), 3.53 (d, J = 3.4 Hz, 4H), 3.23 (d, J = 8.0 Hz, 3H), 2.90 (s, 3H), 2.47 (d, J = 7.9 Hz, 2H), 1.70-1.47 (m, 6H), 1.41-1.32 (m, 2H), 1.26 (dd, J = 22.8, 10.4 Hz, 2H), 1.18-1.08 (m, 2H). MS obsd. (ESI⁺) [(M + H)⁺]: 454.0. |
| 84 | 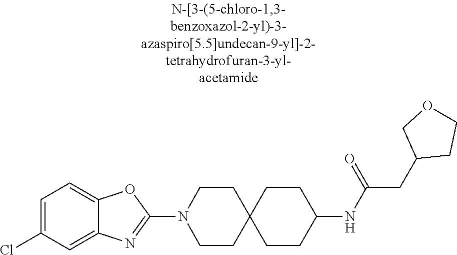 [SMILES: O=C(CC1CCOC1)NC1CCC2(CC1)CCN(c1nc3cc(Cl)ccc3o1)CC2] | AMINE: Int- 19 ACID: 2- tetrahydrofuran- 3-ylacetic acid | ¹H NMR (400 MHz, DMSO-d₆) δ ppm: 7.73 (d, J = 7.8 Hz, 1H), 7.39 (d, J = 8.5 Hz, 1H), 7.30 (d, J = 2.1 Hz, 1H), 7.01 (dd, J = 8.4, 2.2 Hz, 1H), 3.71 (ddd, J = 13.4, 8.2, 6.2 Hz, 2H), 3.64- 3.52 (m, 6H), 3.26 (dd, J = 8.3, 6.5 Hz, 1H), 2.44 (dd, J = 14.4, 7.2 Hz, 1H), 2.12 (dd, J = 7.5, 3.1 Hz, 2H), 1.94 (dtd, J = 12.9, 7.7, 5.4 Hz, 1H), 1.70 (d, J = 13.2 Hz, 2H), 1.58 (dd, J = 17.6, 4.6 Hz, 4H), 1.50-1.40 (m, 3H), 1.36-1.26 (m, 2H), 1.24-1.14 (m, 2H). MS obsd. (ESI⁺) [(M + H)⁺]: 432.2. |
| 85 | 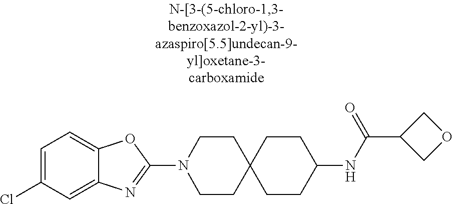 [SMILES: O=C(NC1CCC2(CC1)CCN(c1nc3cc(Cl)ccc3o1)CC2)C1COC1] | AMINE: Int- 19 ACID: oxetane-3- carboxylic acid | ¹H NMR (400 MHz, DMSO-d₆) δ ppm: 7.77 (d, J = 7.4 Hz, 1H), 7.39 (d, J = 8.4 Hz, 1H), 7.30 (s, 1H), 7.01 (d, J = 8.0 Hz, 1H), 4.65-4.55 (m, 3H), 3.60 (s, 5H), 1.63 (dd, J = 51.5, 8.3 Hz, 6H), 1.43 (s, 2H), 1.27 (dd, J = 39.3, 11.7 Hz, 6H). MS obsd. (ESI⁺) [(M + H)⁺]: 404.2. |
| 86 | 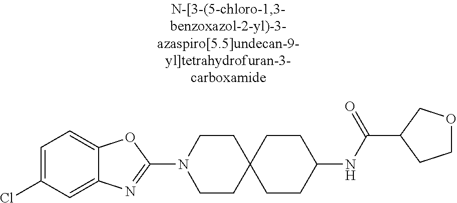 [SMILES: O=C(NC1CCC2(CC1)CCN(c1nc3cc(Cl)ccc3o1)CC2)C1CCOC1] | AMINE: Int- 19 ACID: tetrahydrofuran- 3-carboxylic acid | ¹H NMR (400 MHz, DMSO-d₆) δ ppm: 7.80 (d, J = 7.8 Hz, 1H), 7.39 (d, J = 8.4 Hz, 1H), 7.30 (d, J = 2.1 Hz, 1H), 7.01 (dd, J = 8.4, 2.2 Hz, 1H), 3.83 (t, J = 8.1 Hz, 1H), 3.64 (dddd, J = 25.0, 20.6, 8.1, 6.9 Hz, 7H), 3.33 (s, 2H), 2.94-2.80 (m, 1H), 1.95 (dd, J = 14.7, 7.0 Hz, 2H), 1.70 (d, J = 13.1 Hz, 2H), 1.60-1.52 (m, 3H), 1.45-1.27 (m, 4H), 1.20 (dd, J = 18.6, 7.4 Hz, 2H). MS obsd. (ESI⁺) [(M + H)⁺]: 418.2. |
| 87 | 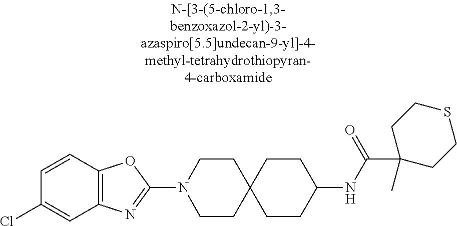 [SMILES: CC1(C(=O)NC2CCC3(CC2)CCN(c2nc4cc(Cl)ccc4o2)CC3)CCSCC1] | AMINE: Int- 19 ACID: 4- methyltetrahydro- thiopyran-4- carboxylic acid | ¹H NMR (400 MHz, CD₃OD) δ ppm: 7.34 (d, J = 8.6 Hz, 1H), 7.27 (d, J = 2.0 Hz, 1H), 7.09 (dd, J = 2.1, 8.6 Hz, 1H), 3.77- 3.69 (m, 5H), 2.73-2.64 (m, 2H), 2.58-2.49 (m, 2H), 2.40- 2.31 (m, 2H), 1.86 (br d, J = 13.4 Hz, 2H), 1.81-1.75 (m, 2H), 1.75-1.69 (m, 2H), 1.64- 1.49 (m, 6H), 1.40-1.34 (m, 2H), 1.14 (s, 3H). MS obsd. (ESI⁺) [(M + H)⁺]: 462.2. |
| 88 | 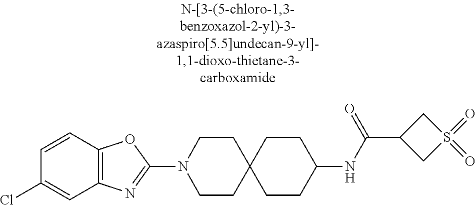 [SMILES: O=C(NC1CCC2(CC1)CCN(c1nc3cc(Cl)ccc3o1)CC2)C1CS(=O)(=O)C1] | AMINE: Int- 19 ACID: 1,1- dioxothietane- 3-carboxylic acid | ¹H NMR (400 MHz, CD₃OD) δ ppm: 7.16 (d, J = 8.6 Hz, 1H), 7.11 (d, J = 2.0 Hz, 1H), 6.91 (dd, J = 2.1, 8.6 Hz, 1H), 4.28- 4.21 (m, 2H), 4.18-4.09 (m, 2H), 3.61-3.54 (m, 5H), 3.19- 3.13 (m, 1H), 1.74-1.57 (m, 6H), 1.45-1.30 (m, 4H), 1.25 (br dd, J = 2.8, 13.0 Hz, 2H). MS obsd. (ESI⁺) [(M + H)⁺]: 452.1. |
| 89 | 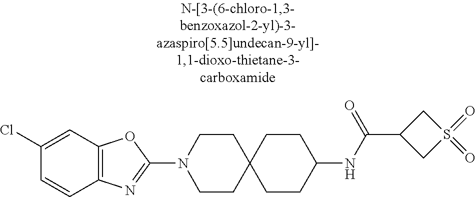 [SMILES: O=C(NC1CCC2(CC1)CCN(c1nc3ccc(Cl)cc3o1)CC2)C1CS(=O)(=O)C1] | AMINE: Int- 20 ACID: 1,1- dioxothietane- 3-carboxylic acid | ¹H NMR (400 MHz, CD₃OD) δ ppm: 7.27 (d, J = 1.5 Hz, 1H), 7.12-7.04 (m, 2H), 4.29-4.21 (m, 2H), 4.18-4.08 (m, 2H), 3.61-3.54 (m, 5H), 1.74-1.58 (m, 6H), 1.46-1.34 (m, 3H), 1.27-1.17 (m, 4H). MS obsd. (ESI⁺) [(M + H)⁺]: 452.1. |
| 90 | 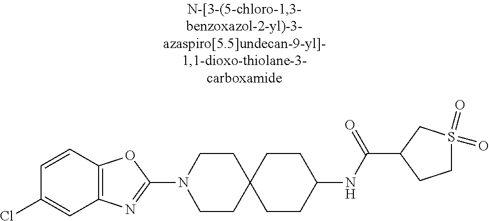 [SMILES: O=C(NC1CCC2(CC1)CCN(c1nc3cc(Cl)ccc3o1)CC2)C1CCS(=O)(=O)C1] | AMINE: Int- 19 ACID: 1,1- dioxothiolane- 3-carboxylic acid | ¹H NMR (400 MHz, CD₃OD) δ ppm: 7.15 (d, J = 8.6 Hz, 1H), 7.10 (d, J = 2.0 Hz, 1H), 6.90 (dd, J = 2.1, 8.4 Hz, 1H), 3.55 (td, J = 3.5, 7.4 Hz, 5H), 3.19- 3.09 (m, 4H), 2.97 (ddd, J = 7.7, 9.3, 13.2 Hz, 1H), 2.37-2.28 (m, 1H), 2.22-2.11 (m, 1H), 1.74-1.62 (m, 4H), 1.62-1.56 (m, 2H), 1.44-1.29 (m, 4H), 1.27-1.16 (m, 2H). MS obsd. (ESI⁺) [(M + H)⁺]: 490.1. |
| 93 | 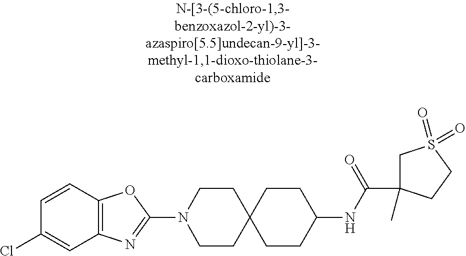 [SMILES: CC1(C(=O)NC2CCC3(CC2)CCN(c2nc4cc(Cl)ccc4o2)CC3)CCS(=O)(=O)C1] | AMINE: Int- 19 ACID: 3- methyl-1,1- dioxo-thiolane- 3-carboxylic acid | ¹H NMR (400 MHz, CD₃OD) & ppm: 7.35 (d, J = 8.6 Hz, 1H), 7.28 (d, J = 2.0 Hz, 1H), 7.11 (dd, J = 2.1, 8.5 Hz, 1H), 3.76- 3.69 (m, 6H), 3.28-3.12 (m, 2H), 3.02 (d, J = 13.7 Hz, 1H), 2.70-2.60 (m, 1H), 2.17 (td, J = 8.1, 13.8 Hz, 1H), 1.86 (br d, J = 13.4 Hz, 2H), 1.81-1.71 (m, 4H), 1.59-1.50 (m, 4H), 1.49 (s, 3H), 1.41-1.32 (m, 2H). MS obsd. (ESI⁺) [(M + H)⁺]: 480.2. |
| 94 | 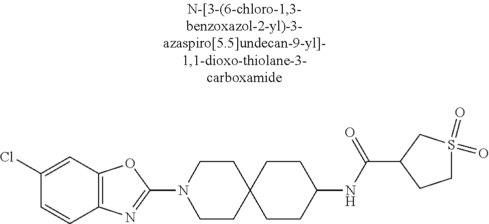 [SMILES: O=C(NC1CCC2(CC1)CCN(c1nc3ccc(Cl)cc3o1)CC2)C1CCS(=O)(=O)C1] | AMINE: Int- 20 ACID: 1,1- dioxothiolane- 3-carboxylic acid | ¹H NMR (400 MHz, CD₃OD) δ ppm: 7.39 (d, J = 1.5 Hz, 1H), 7.23-7.16 (m, 2H), 3.73-3.64 (m, 5H), 3.30-3.22 (m, 4H), 3.09 (ddd, J = 7.7, 9.4, 13.1 Hz, 1H), 2.49-2.38 (m, 1H), 2.32- 2.21 (m, 1H), 1.88-1.70 (m, 6H), 1.59-1.42 (m, 4H), 1.36 (br dd, J = 2.5, 12.9 Hz, 2H). MS obsd. (ESI⁺) [(M + H)⁺]: 466.1. |
| 95 | 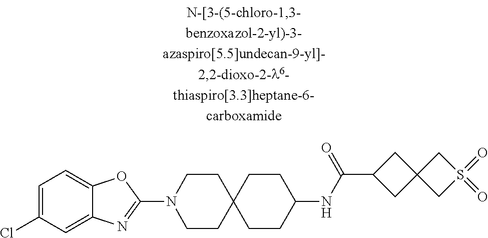 [SMILES: O=C(NC1CCC2(CC1)CCN(c1nc3cc(Cl)ccc3o1)CC2)C1CC2(C1)CS(=O)(=O)C2] | AMINE: Int- 19 ACID: 2,2- dioxo-2-λ⁶- thiaspiro[3.3] heptane-6- carboxylic acid | ¹H NMR (400 MHz, DMSO-d₆) δ ppm: 7.65 (d, J = 7.8 Hz, 1H), 7.39 (d, J = 8.5 Hz, 1H), 7.29 (d, J = 2.1 Hz, 1H), 7.01 (dd, J = 8.4, 2.2 Hz, 1H), 4.24 (s, 2H), 4.13 (s, 2H), 3.73-3.42 (m, 5H), 2.98-2.84 (m, 1H), 2.37 (d, J = 8.2 Hz, 4H), 1.69 (d, J = 12.9 Hz, 2H), 1.57 (d, J = 5.4 Hz, 4H), 1.47-1.38 (m, 2H), 1.32 (d, J = 12.7 Hz, 2H), 1.24- 1.17 (m, 2H). MS obsd. (ESI⁺) [(M + H)⁺]: 492.2. |
| 96 | 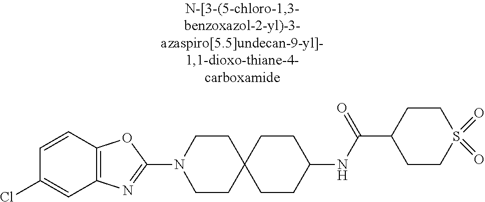 [SMILES: O=C(NC1CCC2(CC1)CCN(c1nc3cc(Cl)ccc3o1)CC2)C1CCS(=O)(=O)CC1] | AMINE: Int- 19 ACID: 1,1- dioxothiane-4- carboxylic acid | ¹H NMR (400 MHz, CD₃OD) δ ppm: 7.18 (d, J = 8.4 Hz, 1H), 7.12 (d, J = 2.1 Hz, 1H), 6.93 (dd, J = 2.1, 8.5 Hz, 1H), 3.62- 3.55 (m, 5H), 3.07-3.00 (m, 4H), 2.43-2.34 (m, 1H), 2.21- 2.04 (m, 5H), 1.98-1.91 (m, 1H), 1.76-1.60 (m, 6H), 1.46- 1.31 (m, 4H), 1.25 (br d, J = 2.0 Hz, 2H). MS obsd. (ESI⁺) [(M + H)⁺]: 480.2 |
| 97 | 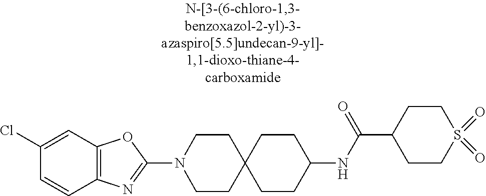 [SMILES: O=C(NC1CCC2(CC1)CCN(c1nc3ccc(Cl)cc3o1)CC2)C1CCS(=O)(=O)CC1] | AMINE: Int- 20 ACID: 1,1- dioxothiane-4- carboxylic acid | ¹H NMR (400 MHz, CD₃OD) δ ppm: 7.24-7.16 (m, 3H), 7.15- 7.10 (m, 2H), 6.97-6.91 (m, 2H), 3.86-3.74 (m, 1H), 3.61 (br dd, J = 4.4, 5.7 Hz, 4H), 3.51 (q, J = 7.1 Hz, 2H), 1.85-1.64 (m, 6H), 1.62-1.40 (m, 4H), 1.33-1.13 (m, 9H). MS obsd. (ESI⁺) [(M + H)⁺]: 480.2. |
| 98 | 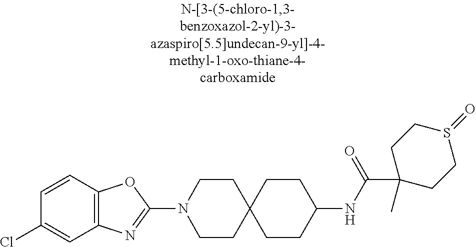 [SMILES: CC1(C(=O)NC2CCC3(CC2)CCN(c2nc4cc(Cl)ccc4o2)CC3)CCS(=O)CC1] | AMINE: Int- 19 ACID: 4- methyl-1-oxo- thiane-4- carboxylic acid | ¹H NMR (400 MHz, CD₃OD) δ ppm: 7.34 (d, J = 8.6 Hz, 1H), 7.28 (d, J = 2.1 Hz, 1H), 7.10 (dd, J = 2.1, 8.5 Hz, 1H), 3.73 (br dd, J = 4.0, 5.4 Hz, 5H), 2.98- 2.90 (m, 2H), 2.76 (td, J = 7.9, 15.3 Hz, 2H), 2.19-2.16 (m, 2H), 1.86 (br d, J = 13.4 Hz, 2H), 1.82-1.70 (m, 4H), 1.59- 1.48 (m, 4H), 1.38-1.28 (m, 4H), 1.23 (s, 3H). MS obsd. (ESI⁺) [(M + H)⁺]: 478.2. |
| 99 | 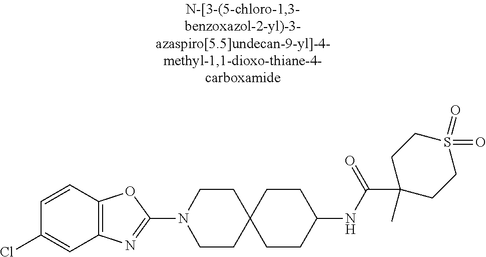 [SMILES: CC1(C(=O)NC2CCC3(CC2)CCN(c2nc4cc(Cl)ccc4o2)CC3)CCS(=O)(=O)CC1] | AMINE: Int- 19 ACID: 4- methyl-1,1- dioxo-thiane- 4-carboxylic acid | ¹H NMR (400 MHz, CD₃OD) δ ppm: 7.33 (d, J = 8.6 Hz, 1H), 7.27 (d, J = 2.0 Hz, 1H), 7.09 (dd, J = 2.1, 8.6 Hz, 1H), 3.80- 3.69 (m, 5H), 3.19-3.10 (m, 2H), 3.07-2.98 (m, 2H), 2.54- 2.47 (m, 2H), 2.09-2.00 (m, 2H), 1.87 (br d, J = 13.0 Hz, 2H), 1.81-1.72 (m, 4H), 1.59- 1.50 (m, 4H), 1.41-1.35 (m, 2H), 1.27 (s, 3H). MS obsd. (ESI⁺) [(M + H)⁺]: 494.2. |
| 100 | 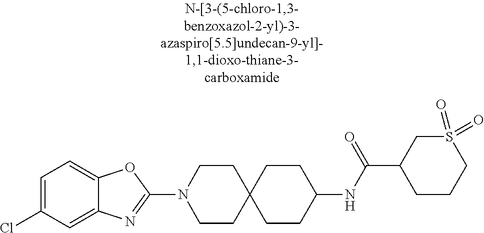 [SMILES: O=C(NC1CCC2(CC1)CCN(c1nc3cc(Cl)ccc3o1)CC2)C1CCCS(=O)(=O)C1] | AMINE: Int- 19 ACID: 1,1- dioxothiane-3- carboxylic acid | ¹H NMR (400 MHz, DMSO-d₆) δ ppm: 7.93 (d, J = 7.8 Hz, 1H), 7.39 (d, J = 8.4 Hz, 1H), 7.30 (d, J = 2.1 Hz, 1H), 7.01 (dd, J = 8.4, 2.1 Hz, 1H), 3.60 (s, 5H), 3.19-2.96 (m, 4H), 2.76 (t, J = 12.0 Hz, 1H), 2.10-2.02 (m, 1H), 1.74 (ddd, J = 20.3, 15.9, 9.6 Hz, 4H), 1.63-1.48 (m, 5H), 1.47-1.39 (m, 2H), 1.38-1.26 (m, 2H), 1.20 (t, J = 11.2 Hz, 2H). MS obsd. (ESI⁺)[(M + H)⁺]: 480.1. |
| 101 | 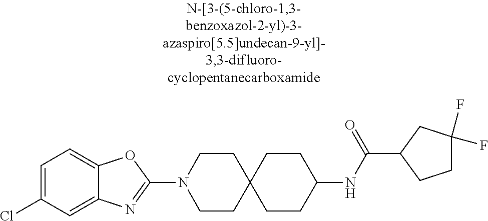 [SMILES: O=C(NC1CCC2(CC1)CCN(c1nc3cc(Cl)ccc3o1)CC2)C1CCC(F)(F)C1] | AMINE: Int- 19 ACID: 3,3- difluorocyclo- pentanecarboxylic acid | ¹H NMR (400 MHz, CD₃OD) δ ppm: 7.24 (dd, J = 20.6, 5.3 Hz, 2H), 7.01 (dd, J = 8.5, 2.1 Hz, 1H), 3.76-3.54 (m, 4H), 2.95- 2.78 (m, 1H), 2.38-2.11 (m, 3H), 2.11-1.98 (m, 2H), 1.98- 1.86 (m, 1H), 1.85-1.62 (m, 6H), 1.57-1.22 (m, 6H). MS obsd. (ESI⁺) [(M + H)⁺]: 452.1. |
| 102 | 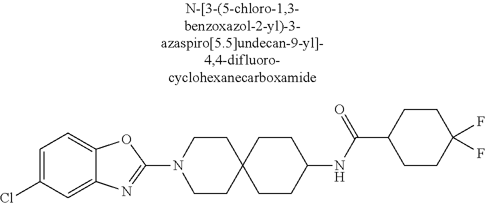 [SMILES: O=C(NC1CCC2(CC1)CCN(c1nc3cc(Cl)ccc3o1)CC2)C1CCC(F)(F)CC1] | AMINE: Int- 19 ACID: 4,4- difluorocyclo- hexanecarboxylic acid | ¹H NMR (400 MHz, CD₃OD) δ ppm: 7.26 (d, J = 8.5 Hz, 1H), 7.21 (d, J = 2.0 Hz, 1H), 7.00 (dd, J = 8.5, 2.1 Hz, 1H), 3.66 (ddd, J = 11.7, 8.0, 3.9 Hz, 5H), 2.31-2.18 (m, 1H), 2.09 (d, J = 7.1 Hz, 2H), 1.90-1.64 (m, 12H), 1.55-1.23 (m, 6H). MS obsd. (ESI⁺) [(M + H)⁺]: 466.1. |
| 103 | 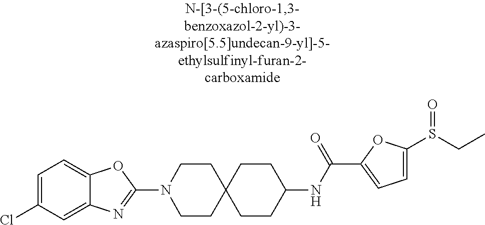 [SMILES: CCS(=O)c1ccc(C(=O)NC2CCC3(CC2)CCN(c2nc4cc(Cl)ccc4o2)CC3)o1] | AMINE: Int- 19 ACID: Int-25 | ¹H NMR (400 MHz, DMSO-d₆) δ ppm: 8.41 (d, J = 8.3 Hz, 1H), 7.40 (d, J = 8.4 Hz, 1H), 7.33- 7.18 (m, 3H), 7.01 (dd, J = 8.4, 2.2 Hz, 1H), 3.86-3.70 (m, 1H), 3.68-3.51 (m, 4H), 3.28 (dd, J = 15.0, 7.0 Hz, 2H), 1.78 (d, J = 13.1 Hz, 2H), 1.60 (dt, J = 24.6, 9.3 Hz, 6H), 1.48-1.38 (m, 2H), 1.23 (td, J = 13.2, 4.0 Hz, 2H), 1.14 (t, J = 7.4 Hz, 3H). MS obsd. (ESI⁺) [(M + H)⁺]: 490.1. |
| 104 | 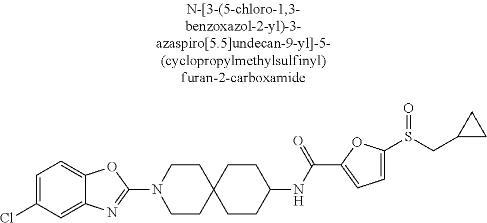 [SMILES: O=C(NC1CCC2(CC1)CCN(c1nc3cc(Cl)ccc3o1)CC2)c1ccc(S(=O)CC2CC2)o1] | AMINE: Int- 19 ACID: Int-28 | ¹H NMR (400 MHz, DMSO-d₆) δ ppm: 8.43 (d, J = 8.3 Hz, 1H), 7.40 (d, J = 8.4 Hz, 1H), 7.30 (dd, J = 3.6, 3.0 Hz, 2H), 7.22 (d, J = 3.6 Hz, 1H), 7.01 (dd, J = 8.4, 2.2 Hz, 1H), 3.85-3.70 (m, 1H), 3.70-3.52 (m, 4H), 3.37 (dd, J = 13.0, 7.0 Hz, 1H), 3.20 (dd, J = 13.0, 7.7 Hz, 1H), 1.78 (d, J = 13.0 Hz, 2H), 1.70-1.50 (m, 6H), 1.47-1.37 (m, 2H), 1.23 (t, J = 10.8 Hz, 2H), 0.90- 0.76 (m, 1H), 0.66-0.48 (m, 2H), 0.46-0.34 (m, 1H), 0.31- 0.19 (m, 1H). MS obsd. (ESI⁺) [(M + H)⁺]: 516.2. |
| 105 | 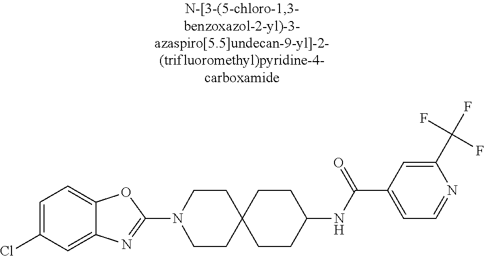 [SMILES: O=C(NC1CCC2(CC1)CCN(c1nc3cc(Cl)ccc3o1)CC2)c1ccnc(C(F)(F)F)c1] | AMINE: Int- 19 ACID: 2- (trifluoromethyl) pyridine-4- carboxylic acid | ¹H NMR (400 MHz, CD₃OD) & ppm: 8.86 (d, J = 5.0 Hz, 1H), 8.18 (s, 1H), 8.01 (d, J = 3.8 Hz, 1H), 7.27 (d, J = 8.5 Hz, 1H), 7.22 (d, J = 2.0 Hz, 1H), 7.02 (dd, J = 8.5, 2.1 Hz, 1H), 3.97- 3.84 (m, 1H), 3.70 (dt, J = 7.3, 3.6 Hz, 4H), 1.94-1.72 (m, 6H), 1.61 (ddd, J = 21.5, 13.4, 5.1 Hz, 4H), 1.44-1.31 (m, 2H). MS obsd. (ESI⁺) [(M + H)⁺]: 493.2. |
| 106 | 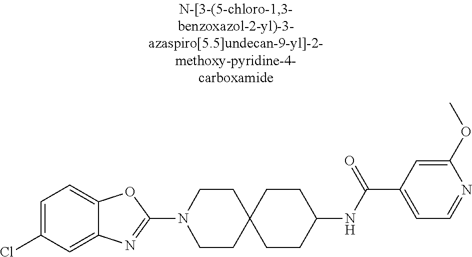 [SMILES: COc1cc(C(=O)NC2CCC3(CC2)CCN(c2nc4cc(Cl)ccc4o2)CC3)ccn1] | AMINE: Int- 19 ACID: 2- methoxypyridine- 4- carboxylic acid | ¹H NMR (400 MHz, CD₃OD) δ ppm: 8.23 (dd, J = 5.3, 0.6 Hz, 1H), 7.30-7.24 (m, 2H), 7.21 (d, J = 2.0 Hz, 1H), 7.14 (d, J = 0.7 Hz, 1H), 7.01 (dd, J = 8.5, 2.1 Hz, 1H), 3.94 (s, 3H), 3.87 (dd, J = 13.4, 9.4 Hz, 1H), 3.75- 3.62 (m, 4H), 1.92-1.70 (m, 6H), 1.58 (dt, J = 11.8, 8.0 Hz, 4H), 1.42-1.32 (m, 2H). MS obsd. (ESI⁺) [(M + H)⁺]: 455.2. |
| 107 | 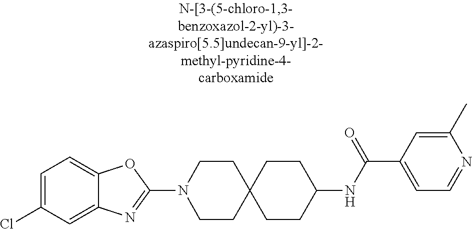 | AMINE: Int- 19 ACID: 2- methylpyridine- 4-carboxylic acid | ¹H NMR (400 MHz, CD₃OD) δ ppm: 8.53 (d, J = 5.3 Hz, 1H), 7.65 (s, 1H), 7.57 (d, J = 4.9 Hz, 1H), 7.27 (d, J = 8.5 Hz, 1H), 7.22 (d, J = 2.1 Hz, 1H), 7.02 (dd, J = 8.5, 2.1 Hz, 1H), 3.90 (t, J = 11.1 Hz, 1H), 3.70 (dt, J = 7.6, 3.8 Hz, 4H), 2.60 (d, J = 5.5 Hz, 3H), 1.82 (dt, J = 11.6, 10.1 Hz, 6H), 1.59 (dt, J = 11.8, 7.8 Hz, 4H), 1.45-1.32 (m, 2H). MS obsd. (ESI⁺) [(M + H)⁺]: 436.2. |
| 108 | 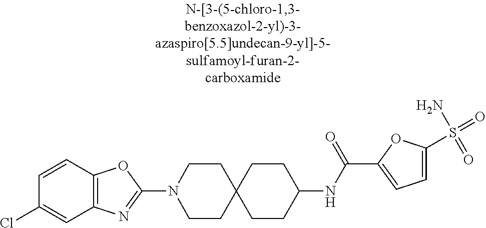 [SMILES: NS(=O)(=O)c1ccc(C(=O)NC2CCC3(CC2)CCN(c2nc4cc(Cl)ccc4o2)CC3)o1] | AMINE: Int- 19 ACID: 5- sulfamoylfuran- 2-carboxylic acid | ¹H NMR (400 MHz, CD₃OD) δ ppm: 7.17 (d, J = 8.4 Hz, 1H), 7.12 (d, J = 2.0 Hz, 1H), 7.06 (d, J = 3.5 Hz, 1H), 6.95-6.89 (m, 2H), 3.83-3.74 (m, 1H), 3.63- 3.56 (m, 4H), 1.82-1.63 (m, 6H), 1.61-1.41 (m, 6H). MS obsd. (ESI⁺) [(M + H)⁺]: 493.1. |
| 109 | 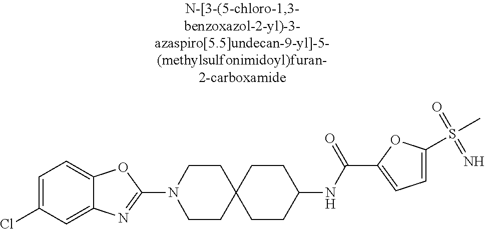 [SMILES: CS(=N)(=O)c1ccc(C(=O)NC2CCC3(CC2)CCN(c2nc4cc(Cl)ccc4o2)CC3)o1] | AMINE: Int- 19 ACID: Int-33 | ¹H NMR (400 MHz, DMSO-d₆) δ ppm: 8.40 (d, J = 8.3 Hz, 1H), 7.39 (dd, J = 8.4, 2.9 Hz, 1H), 7.30 (t, J = 2.2 Hz, 1H), 7.20 (d, J = 3.6 Hz, 1H), 7.14 (d, J = 3.6 Hz, 1H), 7.01 (dt, J = 8.5, 2.1 Hz, 1H), 4.87 (d, J = 1.2 Hz, 1H), 3.77 (dd, J = 11.9, 7.3 Hz, 1H), 3.62 (d, J = 3.7 Hz, 4H), 3.20 (d, J = 1.2 Hz, 3H), 1.78 (t, J = 6.5 Hz, 2H), 1.70-1.51 (m, 6H), 1.48-1.36 (m, 2H), 1.23 (td, J = 13.0, 4.3 Hz, 2H). MS obsd. (ESI⁺) [(M + H)⁺]: 491.1. |
| 110 | 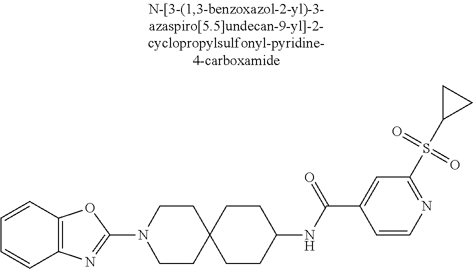 [SMILES: O=C(NC1CCC2(CC1)CCN(c1nc3ccccc3o1)CC2)c1ccnc(S(=O)(=O)C2CC2)c1] | AMINE: Int- 21 ACID: Int-43 | ¹H NMR (400 MHz, CD₃OD) δ ppm: 8.93-8.88 (m, 1H), 8.41- 8.37 (m, 1H), 8.05 (dd, J = 1.7, 5.0 Hz, 1H), 7.40 (d, J = 7.9 Hz, 1H), 7.36-7.30 (m, 1H), 7.25 (dt, J = 1.0, 7.6 Hz, 1H), 7.17- 7.11 (m, 1H), 3.94 (dt, J = 3.9, 7.7 Hz, 1H), 3.75 (br dd, J = 4.4, 5.9 Hz, 4H), 2.94 (tt, J = 4.8, 7.9 Hz, 1H), 1.96-1.86 (m, 4H), 1.84-1.78 (m, 2H), 1.73-1.57 (m, 4H), 1.41 (dt, J = 3.7, 13.5 Hz, 2H), 1.33-1.28 (m, 2H), 1.19-1.11 (m, 2H). MS obsd. (ESI⁺) [(M + H)⁺]: 494.1. |
| 111 | 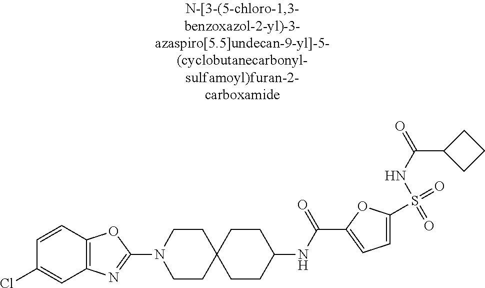 [SMILES: O=C(NC1CCC2(CC1)CCN(c1nc3cc(Cl)ccc3o1)CC2)c1ccc(S(=O)(=O)NC(=O)C2CCC2)o1] | AMINE: Example 108 ACID: cyclobutane- carboxylic acid | ¹H NMR (400 MHz, DMSO-d₆) δ ppm: 3.74 (br dd, J = 3.2, 11.7 Hz, 1H), 3.66-3.61 (m, 4H), 3.17 (quin, J = 8.3 Hz, 1H), 2.09- 2.01 (m, 4H), 1.94-1.84 (m, 1H), 1.82-1.71 (m, 3H), 1.68- 1.60 (m, 4H), 1.59-1.48 (m, 2H), 1.46-1.40 (m, 2H), 1.28- 1.20 (m, 2H). MS obsd. (ESI⁺) [(M + H)⁺]: 575.1. |
| 112 | 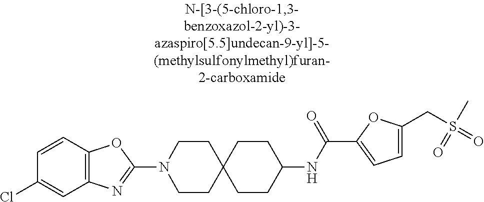 [SMILES: CS(=O)(=O)Cc1ccc(C(=O)NC2CCC3(CC2)CCN(c2nc4cc(Cl)ccc4o2)CC3)o1] | AMINE: Int- 19 ACID: Int-32 | ¹H NMR (400 MHz, CD₃OD) δ ppm: 7.32 (d, J = 8.4 Hz, 1H), 7.26 (d, J = 2.1 Hz, 1H), 7.15 (d, J = 3.5 Hz, 1H), 7.07 (dd, J = 2.1, 8.6 Hz, 1H), 6.72 (d, J = 3.5 Hz, 1H), 4.68-4.63 (m, 2H), 3.89 (tt, J = 4.2, 11.4 Hz, 1H), 3.76-3.70 (m, 4H), 3.03 (s, 3H), 1.94-1.78 (m, 6H), 1.71- 1.54 (m, 4H), 1.39 (dt, J = 3.4, 13.4 Hz, 2H). MS obsd. (ESI⁺) [(M + H)⁺]: 506.1. |
| 113 | 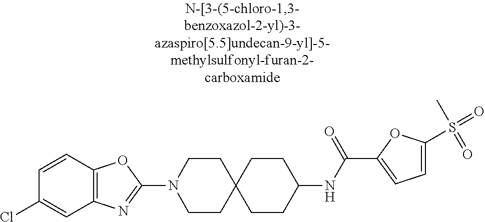 [SMILES: CS(=O)(=O)c1ccc(C(=O)NC2CCC3(CC2)CCN(c2nc4cc(Cl)ccc4o2)CC3)o1] | AMINE: Int- 19 ACID: Int-24 | ¹H NMR (400 MHz, CD₃OD) δ ppm: 7.23 (d, J = 8.6 Hz, 1H), 7.20 (d, J = 3.7 Hz, 1H), 7.16 (d, J = 2.0 Hz, 1H), 7.12 (d, J = 3.7 Hz, 1H), 6.99 (dd, J = 2.1, 8.6 Hz, 1H), 3.85-3.75 (m, 1H), 3.65-3.59 (m, 4H), 1.82-1.67 (m, 6H), 1.62-1.44 (m, 5H), 1.32-1.17 (m, 4H). MS obsd. (ESI⁺) [(M + H)⁺]: 492.1. |
| 114 | 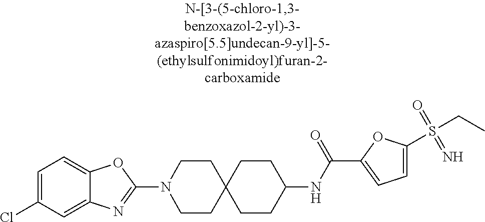 [SMILES: CCS(=N)(=O)c1ccc(C(=O)NC2CCC3(CC2)CCN(c2nc4cc(Cl)ccc4o2)CC3)o1] | AMINE: Int- 19 ACID: Int-34 | ¹H NMR (400 MHz, DMSO-d₆) δ ppm: 8.39 (d, J = 8.3 Hz, 1H), 7.40 (d, J = 8.5 Hz, 1H), 7.30 (d, J = 2.1 Hz, 1H), 7.22 (d, J = 3.6 Hz, 1H), 7.16 (d, J = 3.6 Hz, 1H), 7.01 (dd, J = 8.4, 2.2 Hz, 1H), 4.89 (s, 1H), 3.83-3.71 (m, 1H), 3.62 (d, J = 3.5 Hz, 4H), 3.30-3.22 (m, 2H), 1.78 (d, J = 14.5 Hz, 2H), 1.66-1.53 (m, 6H), 1.46-1.39 (m, 2H), 1.27-1.19 (m, 2H), 1.14 (t, J = 7.4 Hz, 3H). MS obsd. (ESI⁺) [(M + H)⁺]: 505.1. |
| 115 | 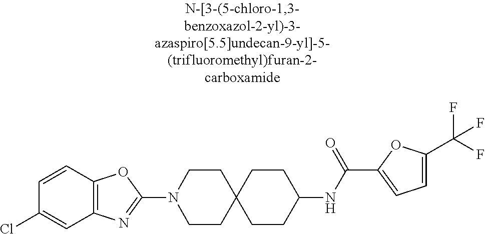 [SMILES: O=C(NC1CCC2(CC1)CCN(c1nc3cc(Cl)ccc3o1)CC2)c1ccc(C(F)(F)F)o1] | AMINE: Int- 19 ACID: 5- (trifluoromethyl) furan-2- carboxylic acid | ¹H NMR (400 MHz, CD₃OD) & ppm: 7.31 (d, J = 8.4 Hz, 1H), 7.26 (d, J = 2.1 Hz, 1H), 7.21 (dd, J = 0.7, 3.6 Hz, 1H), 7.16- 7.12 (m, 1H), 7.06 (dd, J = 2.1, 8.6 Hz, 1H), 3.96-3.86 (m, 1H), 3.77-3.69 (m, 4H), 1.93-1.76 (m, 6H), 1.72-1.61 (m, 2H), 1.58-1.53 (m, 2H), 1.37 (dt, J = 3.6, 13.4 Hz, 2H). MS obsd. (ESI⁺) [(M + H)⁺]: 482.2. |
| 116 | 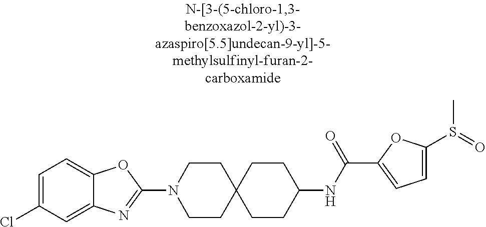 [SMILES: CS(=O)c1ccc(C(=O)NC2CCC3(CC2)CCN(c2nc4cc(Cl)ccc4o2)CC3)o1] | AMINE: Int- 19 ACID: Int-23 | ¹H NMR (400 MHz, DMSO-d₆) δ ppm: 8.44 (d, J = 8.3 Hz, 1H), 7.40 (d, J = 8.4 Hz, 1H), 7.30 (d, J = 2.1 Hz, 1H), 7.26 (d, J = 3.6 Hz, 1H), 7.22 (d, J = 3.6 Hz, 1H), 7.01 (dd, J = 8.4, 2.2 Hz, 1H), 3.85-3.71 (m, 1H), 3.63 (dd, J = 7.1, 3.7 Hz, 4H), 3.05 (s, 3H), 1.78 (d, J = 13.0 Hz, 2H), 1.61 (dt, J = 25.2, 9.4 Hz, 6H), 1.48-1.37 (m, 2H), 1.23 (td, J = 13.2, 4.3 Hz, 2H). MS obsd. (ESI⁺) [(M + H)⁺]: 476.1. |
| 117 | 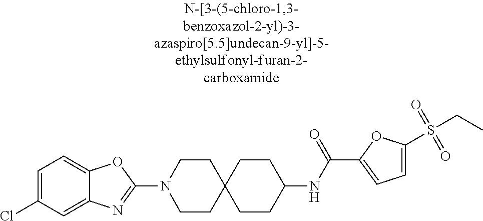 [SMILES: CCS(=O)(=O)c1ccc(C(=O)NC2CCC3(CC2)CCN(c2nc4cc(Cl)ccc4o2)CC3)o1] | AMINE: Int- 19 ACID: Int-26 | ¹H NMR (400 MHz, DMSO-d₆) δ ppm: 8.49 (d, J = 8.2 Hz, 1H), 7.40 (t, J = 6.3 Hz, 2H), 7.30 (dd, J = 4.6, 2.9 Hz, 2H), 7.01 (dd, J = 8.4, 2.2 Hz, 1H), 3.84- 3.72 (m, 1H), 3.65-3.60 (m, 4H), 3.46 (q, J = 7.3 Hz, 2H), 1.78 (d, J = 13.1 Hz, 2H), 1.69- 1.50 (m, 6H), 1.48-1.38 (m, 2H), 1.25 (dd, J = 13.2, 3.6 Hz, 2H), 1.18 (t, J = 7.4 Hz, 3H). MS obsd. (ESI⁺) [(M + H)⁺]: 506.1. |
| 118 | 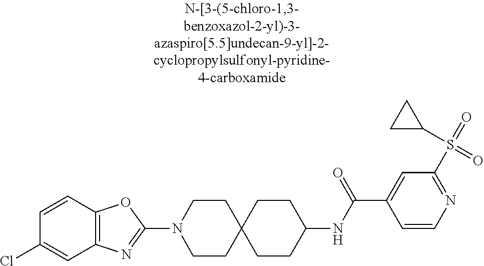 [SMILES: O=C(NC1CCC2(CC1)CCN(c1nc3cc(Cl)ccc3o1)CC2)c1ccnc(S(=O)(=O)C2CC2)c1] | AMINE: Int- 19 ACID: Int-43 | ¹H NMR (400 MHz, CD₃OD) δ ppm: 8.79 (dd, J = 0.7, 5.0 Hz, 1H), 8.27 (dd, J = 0.7, 1.6 Hz, 1H), 7.93 (dd, J = 1.7, 5.0 Hz, 1H), 7.17 (d, J = 8.4 Hz, 1H), 7.12 (d, J = 2.1 Hz, 1H), 6.91 (dd, J = 2.1, 8.5 Hz, 1H), 3.82 (ddd, J = 4.3, 7.3, 11.4 Hz, 1H), 3.60 (td, J = 3.7, 7.6 Hz, 4H), 2.82 (tt, J = 4.8, 8.0 Hz, 1H), 1.82-1.73 (m, 4H), 1.69-1.64 (m, 2H), 1.61-1.48 (m, 2H), 1.47-1.41 (m, 2H), 1.34-1.22 (m, 3H), 1.10-0.98 (m, 3H). MS obsd. (ESI⁺) [(M + H)⁺]: 529.2. |
| 119 | 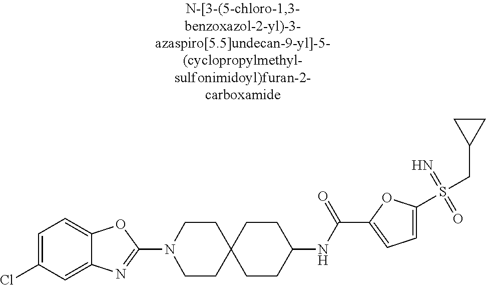 [SMILES: N=S(=O)(CC1CC1)c1ccc(C(=O)NC2CCC3(CC2)CCN(c2nc4cc(Cl)ccc4o2)CC3)o1] | AMINE: Int- 19 ACID: Int-35 | ¹H NMR (400 MHz, DMSO-d₆) δ ppm: 8.37 (d, J = 8.1 Hz, 1H), 7.39 (dd, J = 8.4, 2.5 Hz, 1H), 7.30 (t, J = 2.3 Hz, 1H), 7.25- 7.20 (m, 1H), 7.18-7.13 (m, 1H), 7.02-6.99 (m, 1H), 4.86 (s, 1H), 3.82-3.71 (m, 1H), 3.62 (s, 4H), 3.27-3.21 (m, 2H), 1.77 (d, J = 12.2 Hz, 2H), 1.62-1.52 (m, 6H), 1.43 (s, 2H), 1.22 (t, J = 11.1 Hz, 2H), 1.03-0.91 (m, 1H), 0.45 (t, J = 9.0 Hz, 2H), 0.10 (t, J = 5.6 Hz, 2H) MS obsd. (ESI⁺) [(M + H)⁺]: 531.1. |
| 120 | 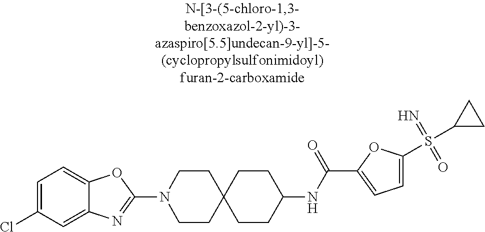 [SMILES: N=S(=O)(c1ccc(C(=O)NC2CCC3(CC2)CCN(c2nc4cc(Cl)ccc4o2)CC3)o1)C1CC1] | AMINE: Int- 19 ACID: Int-44 | ¹H NMR (400 MHz, DMSO-d₆) δ ppm: 8.38 (d, J = 8.3 Hz, 1H), 7.40 (d, J = 8.4 Hz, 1H), 7.30 (d, J = 2.1 Hz, 1H), 7.22 (d, J = 3.6 Hz, 1H), 7.11 (d, J = 3.6 Hz, 1H), 7.01 (dd, J = 8.4, 2.2 Hz, 1H), 4.94 (s, 1H), 3.77 (dd, J = 18.9, 10.8 Hz, 1H), 3.62 (d, J = 3.5 Hz, 4H), 2.84-2.70 (m, 1H), 1.78 (d, J = 13.0 Hz, 2H), 1.71- 1.49 (m, 6H), 1.49-1.37 (m, 2H), 1.27-0.95 (m, 6H). MS obsd. (ESI⁺)[(M + H)⁺]: 517.2. |
| 121 | 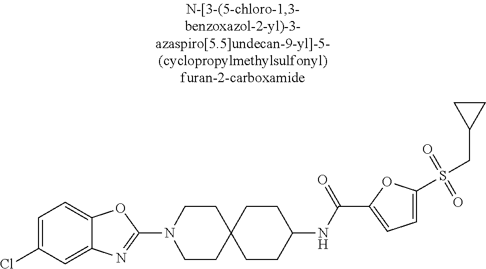 [SMILES: O=C(NC1CCC2(CC1)CCN(c1nc3cc(Cl)ccc3o1)CC2)c1ccc(S(=O)(=O)CC2CC2)o1] | AMINE: Int- 19 ACID: Int-29 | ¹H NMR (400 MHz, DMSO-d₆) δ ppm: 8.47 (d, J = 8.2 Hz, 1H), 7.40 (t, J = 2.8 Hz, 2H), 7.30 (t, J = 2.8 Hz, 2H), 7.01 (dd, J = 8.4, 2.2 Hz, 1H), 3.84-3.72 (m, 1H), 3.71-3.52 (m, 4H), 3.44 (d, J = 7.2 Hz, 2H), 1.78 (d, J = 13.1 Hz, 2H), 1.66-1.52 (m, 6H), 1.49-1.36 (m, 2H), 1.27- 1.20 (m, 2H), 1.00-0.88 (m, 1H), 0.54-0.46 (m, 2H), 0.20- 0.12 (m, 2H). MS obsd. (ESI⁺) [(M + H)⁺]: 532.1. |
| 122 | 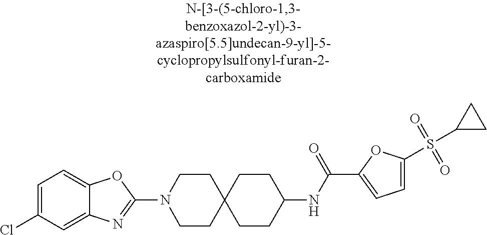 [SMILES: O=C(NC1CCC2(CC1)CCN(c1nc3cc(Cl)ccc3o1)CC2)c1ccc(S(=O)(=O)C2CC2)o1] | AMINE: Int- 19 ACID: Int-27 | ¹H NMR (400 MHz, DMSO-d₆) δ ppm: 8.49 (d, J = 8.2 Hz, 1H), 7.38 (dd, J = 11.6, 6.1 Hz, 2H), 7.30 (dd, J = 3.8, 3.1 Hz, 2H), 7.01 (dd, J = 8.4, 2.1 Hz, 1H), 3.78 (d, J = 7.8 Hz, 1H), 3.68- 3.56 (m, 4H), 2.98 (ddd, J = 12.7, 7.8, 4.8 Hz, 1H), 1.78 (d, J = 13.0 Hz, 2H), 1.70-1.51 (m, 6H), 1.49-1.40 (m, 2H), 1.28- 1.13 (m, 6H). MS obsd. (ESI⁺) [(M + H)⁺]: 518.2. |

### Example 21

### Ethyl N-[2-(5-chloro-1,3-benzoxazol-2-yl)-2-azaspiro[3.3]heptan-6-yl]carbamate

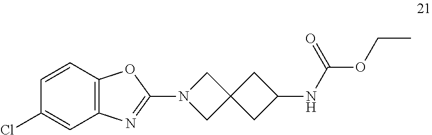 [SMILES: CCOC(=O)NC1CC2(C1)CN(c1nc3cc(Cl)ccc3o1)C2]

The title compound was prepared according to the following scheme:

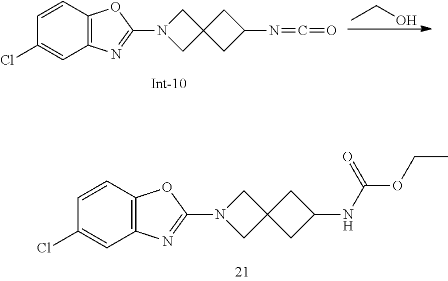

Ethanol (46 mg, 1.0 mmol) was added to a solution of 5-chloro-2-(6-isocyanato-2-azaspiro[3.3]heptan-2-yl)-1,3-benzoxazole (Int-10, 95 mg, 0.33 mmol) in CH₂Cl₂ (5 mL) at r.t. The mixture was stirred at r.t. for 2 h. LC-MS detection indicated that the starting materials were consumed. The mixture was concentrated. The residue was purified by prep-HPLC to afford ethyl N-[2-(5-chloro-1,3-benzoxazol-2-yl)-2-azaspiro[3.3]heptan-6-yl]carbamate (35 mg). MS obsd. (ESI⁺)[(M+H)⁺]: 336.1. ¹H NMR (400 MHz, DMSO-d₆) δ ppm: 7.41 (t, J=7.2 Hz, 2H), 7.33 (d, J=2.1 Hz, 1H), 7.04 (dd, J=8.5, 2.2 Hz, 1H), 4.25 (s, 2H), 4.13 (s, 2H), 3.93 (dt, J=25.0, 7.5 Hz, 3H), 2.56-2.52 (m, 1H), 2.49-2.45 (m, 1H), 2.13 (td, J=9.1, 2.6 Hz, 2H), 1.14 (t, J=7.1 Hz, 3H).

### Example 22

### Isobutyl N-[2-(5-chloro-1,3-benzoxazol-2-yl)-2-azaspiro[3.3]heptan-6-yl]carbamate

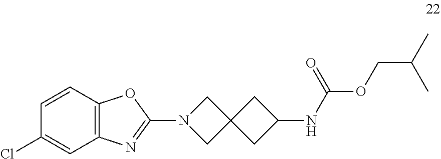 [SMILES: CC(C)COC(=O)NC1CC2(C1)CN(c1nc3cc(Cl)ccc3o1)C2]

The title compound was prepared in analogy to the procedure described for the preparation of Example 21, by using 2-methylpropan-1-ol instead of ethanol. MS obsd. (ESI⁺)[(M+H)⁺]: 336.1. ¹H NMR (400 MHz, DMSO-d₆) δ ppm: 7.42 (d, J=8.5 Hz, 2H), 7.33 (d, J=2.1 Hz, 1H), 7.04 (dd, J=8.5, 2.2 Hz, 1H), 4.19 (d, J=46.7 Hz, 3H), 3.96-3.65 (m, 4H), 2.55-2.52 (m, 1H), 2.48-2.32 (m, 1H), 2.18-2.02 (m, 2H), 1.81 (s, 1H), 0.86 (t, J=6.1 Hz, 6H).

### Example 23

### 3-[2-(5-Chloro-1,3-benzoxazol-2-yl)-2-azaspiro[3.3]heptan-6-yl]-1,1-dimethyl-urea

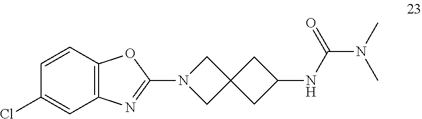 [SMILES: CN(C)C(=O)NC1CC2(C1)CN(c1nc3cc(Cl)ccc3o1)C2]

The title compound was prepared in analogy to the procedure described for the preparation of Example 21, by using N-methylmethanamine instead of ethanol. MS obsd. (ESI⁺)[(M+H)⁺]: 335.1. ¹H NMR (400 MHz, DMSO-d₆) δ ppm: 7.41 (d, J=8.5 Hz, 1H), 7.32 (d, J=2.1 Hz, 1H), 7.04 (dd, J=8.5, 2.1 Hz, 1H), 6.39 (d, J=7.4 Hz, 1H), 4.25 (s, 2H), 4.13 (s, 2H), 4.00 (dd, J=15.9, 8.0 Hz, 1H), 2.75 (s, 6H), 2.49-2.44 (m, 2H), 2.27-2.10 (m, 2H).

### Example 24

### 1-[2-(5-Chloro-1,3-benzoxazol-2-yl)-2-azaspiro[3.3]heptan-6-yl]-3-ethyl-urea

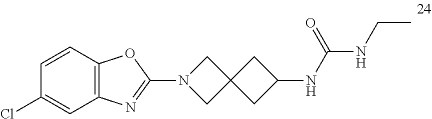 [SMILES: CCNC(=O)NC1CC2(C1)CN(c1nc3cc(Cl)ccc3o1)C2]

The title compound was prepared in analogy to the procedure described for the preparation of Example 21, by using ethanamine instead of ethanol. MS obsd. (ESI⁺)[(M+H)⁺]: 335.1. ¹H NMR (400 MHz, DMSO-d₆) δ ppm: 7.40 (d, J=8.5 Hz, 1H), 7.32 (d, J=2.1 Hz, 1H), 7.03 (dd, J=8.5, 2.1 Hz, 1H), 6.07 (d, J=7.9 Hz, 1H), 5.75 (t, J=5.5 Hz, 1H), 4.23 (s, 2H), 4.11 (s, 2H), 3.95 (dd, J=16.0, 8.0 Hz, 1H), 3.07-2.87 (m, 2H), 2.52-2.48 (m, 2H), 2.03 (td, J=9.1, 2.7 Hz, 2H), 0.96 (t, J=7.2 Hz, 3H).

### Example 47 and Example 63

### N-[2-(5-chloro-1,3-benzoxazol-2-yl)-2-azaspiro[3.3]heptan-6-yl]-5-[(S)-methylsulfonimidoyl]furan-2-carboxamide

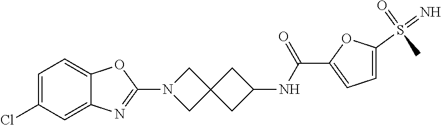 [SMILES: C[S@](=N)(=O)c1ccc(C(=O)NC2CC3(C2)CN(c2nc4cc(Cl)ccc4o2)C3)o1]

N-[2-(5-chloro-1,3-benzoxazol-2-yl)-2-azaspiro[3.3]heptan-6-yl]-5-[(R)-methylsulfonimidoyl]furan-2-carboxamide

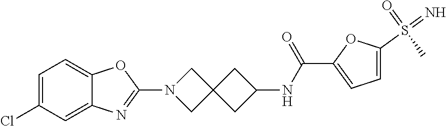 [SMILES: C[S@@](=N)(=O)c1ccc(C(=O)NC2CC3(C2)CN(c2nc4cc(Cl)ccc4o2)C3)o1]

The Example 47 and Example 63 were obtained through SFC [Instrument: Thar 200 preparative SFC (SFC-7), Phenomenex Lux Cellulose-2, 300×50 mm I.D., 10 μm; Mobile phase: A for CO₂ and B for MEOH; Gradient: B 50%; Flow rate: 200 mL/min; Back pressure: 100 bar; Column temperature: 38° C.] chiral separation of N-[2-(5-chloro-1,3-benzoxazol-2-yl)-2-azaspiro[3.3]heptan-6-yl]-5-(methylsulfonimidoyl)furan-2-carboxamide (Example 40).

Example 47: MS obsd. (ESI⁺)[(M+H)⁺]: 435.1. ¹H NMR (400 MHz, CDCl₃) δ ppm: 7.34 (d, J=2.0 Hz, 1H), 7.21-7.14 (m, 3H), 7.02 (dd, J=8.5, 2.1 Hz, 1H), 6.82 (d, J=7.5 Hz, 1H), 4.50 (dd, J=16.2, 8.2 Hz, 1H), 4.37 (s, 2H), 4.29 (s, 2H), 3.25 (s, 3H), 2.84-2.77 (m, 2H), 2.41-2.33 (m, 2H).

Example 63: MS obsd. (ESI⁺)[(M+H)⁺]: 435.1. ¹H NMR (400 MHz, DMSO-d₆) δ ppm: 8.81 (d, J=7.5 Hz, 1H), 7.43 (d, J=8.5 Hz, 1H), 7.34 (d, J=2.1 Hz, 1H), 7.21 (d, J=3.6 Hz, 1H), 7.14 (d, J=3.6 Hz, 1H), 7.05 (dd, J=8.5, 2.1 Hz, 1H), 4.87 (s, 1H), 4.39-4.27 (m, 3H), 4.19 (s, 2H), 3.20 (d, J=1.1 Hz, 3H), 2.66-2.57 (m, 2H), 2.42-2.34 (m, 2H).

### Example 48

### N-[2-(6-chlorooxazolo[5,4-b]pyridin-2-yl)-2-azaspiro[3.3]heptan-6-yl]-5-methylsulfonyl-furan-2-carboxamide

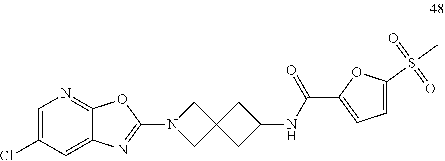 [SMILES: CS(=O)(=O)c1ccc(C(=O)NC2CC3(C2)CN(c2nc4cc(Cl)cnc4o2)C3)o1]

The title compound was prepared according to the following scheme:

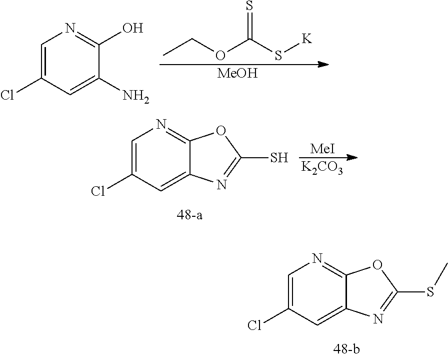

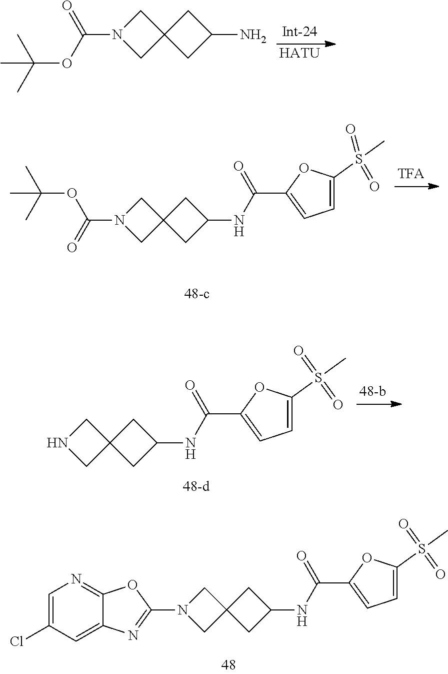

### Step 1: Preparation of 6-chlorooxazolo[5,4-b]pyridine-2-thiol (48-a)

Potassium ethylxanthogenate (685 mg, 4.83 mmol) was added to a solution of 3-amino-5-chloro-pyridin-2-ol (720 mg, 5.0 mmol) in MeOH (20 mL) at r.t. and the mixture was stirred at 100° C. for 2 h. LC-MS detection indicated that the starting materials were consumed. The mixture was concentrated. The residue was purified by chromatograph on silica gel column to afford 6-chlorooxazolo[5,4-b]pyridine-2-thiol (929 mg). MS obsd. (ESI⁺) [(M+H)⁺]: 187.0.

### Step 2: Preparation of 6-chloro-2-methylsulfanyl-oxazolo[5,4-b]pyridine (48-b)

MeI (1.75 g, 11.0 mmol) was added to a solution of potassium carbonate (440 mg, 3.19 mmol) and 6-chlorooxazolo[5,4-b]pyridine-2-thiol (300 mg, 1.61 mmol) in DMF (5 mL) at r.t. and the mixture was stirred for further 2 h. LC-MS detection indicated that the starting materials were consumed. The mixture was concentrated. The residue was purified by chromatograph on silica gel column to afford 6-chloro-2-methylsulfanyl-oxazolo[5,4-b]pyridine (322 mg). MS obsd. (ESI⁺) [(M+H)⁺]: 201.0.

### Step 3: Preparation of tert-butyl 6-[(5-methylsulfonylfuran-2-carbonyl)amino]-2-azaspiro[3.3]heptane-2-carboxylate (48-c)

HATU (684 mg, 1.8 mmol) was added to a solution of TEA (429 mg, 4.24 mmol), 5-methylsulfonylfuran-2-carboxylic acid (Int-24, 274 mg, 1.44 mmol) and 6-amino-2-aza-spiro[3.3]heptane-2-carboxylic acid tert-butyl ester (300 mg, 1.41 mmol) in CH₂Cl₂ (10 mL) at r.t. Then the mixture was stirred at r.t. overnight. LC-MS detection indicated that the reaction was completed. The mixture was concentrated and the residue was purified by silica gel column to afford tert-butyl 6-[(5-methylsulfonylfuran-2-carbonyl)amino]-2-azaspiro[3.3]heptane-2-carboxylate (400 mg). MS obsd. (ESI⁺) [(M+H)⁺]: 385.1.

### Step 4: Preparation of N-(2-azaspiro[3.3]heptan-6-yl)-5-methylsulfonyl-furan-2-carboxamide (48-d)

TFA (69 mg, 0.6 mmol) was added to a solution of tert-butyl 6-[(5-methylsulfonylfuran-2-carbonyl)amino]-2-azaspiro[3.3]heptane-2-carboxylate (78 mg, 0.2 mmol) in CH₂Cl₂ (5 mL) and the mixture was stirred overnight at r.t. LC-MS detection indicated that the starting materials were consumed. The mixture was concentrated and the residue was separated between CH₂Cl₂ and NaHCO₃ solution. The organic phase was concentrated to afford crude N-(2-azaspiro[3.3]heptan-6-yl)-5-methylsulfonyl-furan-2-carboxamide (57 mg). MS obsd. (ESI⁺) [(M+H)⁺]: 285.1.

### Step 5: Preparation of N-[2-(6-chlorooxazolo[5,4-b]pyridin-2-yl)-2-azaspiro[3.3]heptan-6-yl]-5-methylsulfonyl-furan-2-carboxamide (48)

DIPEA (97 mg, 0.75 mmol) was added to a solution of 6-chloro-2-methylsulfanyl-oxazolo[5,4-b]pyridine (48-b, 50 mg, 0.25 mmol) and N-(2-azaspiro[3.3]heptan-6-yl)-5-methylsulfonyl-furan-2-carboxamide (48-d, 85 mg, 0.30 mmol) in DMF (5 mL) at r.t. and the mixture was stirred at 100° C. overnight. LC-MS detection indicated that the starting materials were consumed. The mixture was concentrated. The residue was purified by prep-HPLC to afford N-[2-(6-chlorooxazolo[5,4-b]pyridin-2-yl)-2-azaspiro[3.3]heptan-6-yl]-5-methylsulfonyl-furan-2-carboxamide (27 mg). MS obsd. (ESI⁺)[(M+H)⁺]: 436.9. ¹H NMR (400 MHz, DMSO-d₆) δ ppm: 8.90 (d, J=7.4 Hz, 1H), 7.92 (d, J=2.2 Hz, 1H), 7.79 (d, J=2.2 Hz, 1H), 7.38 (d, J=3.7 Hz, 1H), 7.27 (d, J=3.7 Hz, 1H), 4.44-4.29 (m, 3H), 4.26 (d, J=15.8 Hz, 2H), 3.39 (s, 3H), 2.68-2.60 (m, 2H), 2.38 (dd, J=15.5, 5.9 Hz, 2H).

### Example 91 and Example 92

### (3S)—N-[3-(5-chloro-1,3-benzoxazol-2-yl)-3-azaspiro[5.5]undecan-9-yl]-1,1-dioxo-thiolane-3-carboxamide

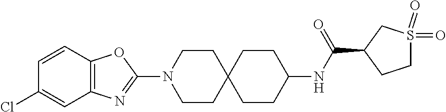 [SMILES: O=C(NC1CCC2(CC1)CCN(c1nc3cc(Cl)ccc3o1)CC2)[C@@H]1CCS(=O)(=O)C1]

### (3R)—N-[3-(5-chloro-1,3-benzoxazol-2-yl)-3-azaspiro[5.5]undecan-9-yl]-1,1-dioxo-thiolane-3-carboxamide

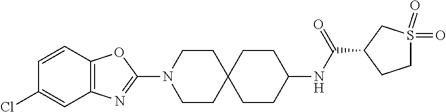 [SMILES: O=C(NC1CCC2(CC1)CCN(c1nc3cc(Cl)ccc3o1)CC2)[C@H]1CCS(=O)(=O)C1]

The Example 91 and Example 92 were obtained through SFC [Instrument: Thar 200 preparative SFC (SFC-7), Phenomenex Lux Cellulose-2, 300×50 mm I.D., 10 μm; Mobile phase: A for CO₂ and B for MEOH; Gradient: B 50%; Flow rate: 200 mL/min; Back pressure: 100 bar; Column temperature: 38° C.] chiral separation of N-[3-(5-chloro-1,3-benzoxazol-2-yl)-3-azaspiro[5.5]undecan-9-yl]-1,1-dioxo-thiolane-3-carboxamide (Example 90).

Example 91: MS obsd. (ESI⁺)[(M+H)⁺]: 466.1. ¹H NMR (400 MHz, CD₃OD) δ ppm: 7.28 (d, J=8.6 Hz, 1H), 7.23 (d, J=2.1 Hz, 1H), 7.03 (dd, J=2.1, 8.4 Hz, 1H), 3.73-3.64 (m, 5H), 3.31-3.22 (m, 4H), 3.09 (ddd, J=7.7, 9.4, 13.1 Hz, 1H), 2.48-2.39 (m, 1H), 2.33-2.21 (m, 1H), 1.87-1.70 (m, 6H), 1.59-1.51 (m, 2H), 1.50-1.41 (m, 2H), 1.40-1.34 (m, 2H).

Example 92: MS obsd. (ESI⁺)[(M+H)⁺]: 466.1. ¹H NMR (400 MHz, CD₃OD) δ ppm: 7.28 (d, J=8.6 Hz, 1H), 7.23 (d, J=2.1 Hz, 1H), 7.03 (dd, J=2.1, 8.4 Hz, 1H), 3.74-3.64 (m, 5H), 3.31-3.21 (m, 4H), 3.09 (ddd, J=7.7, 9.4, 13.1 Hz, 1H), 2.48-2.40 (m, 1H), 2.32-2.20 (m, 1H), 1.87-1.70 (m, 6H), 1.57-1.51 (m, 2H), 1.48 (br d, J=13.2 Hz, 2H), 1.40-1.34 (m, 2H).

### Example 123

### N-[2-(5-chloro-1,3-benzoxazol-2-yl)-3,3a,4,5,6,6a-hexahydro-1H-cyclopenta[c]pyrrol-5-yl]-5-(trifluoromethyl)furan-2-carboxamide

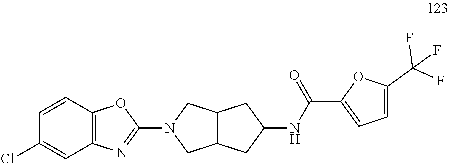 [SMILES: O=C(NC1CC2CN(c3nc4cc(Cl)ccc4o3)CC2C1)c1ccc(C(F)(F)F)o1]

The title compound was prepared according to the following scheme:

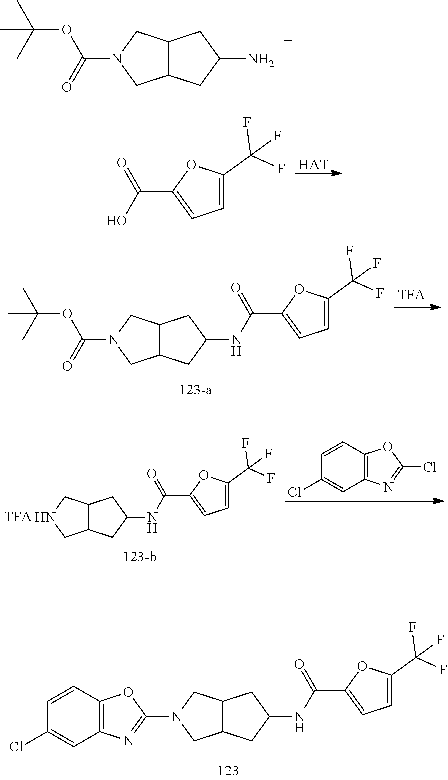

### Step 1: Preparation of tert-butyl 5-[[5-(trifluoromethyl)furan-2-carbonyl]amino]-3,3a,4,5,6,6a-hexahydro-1H-cyclopenta[c]pyrrole-2-carboxylate (123-a)

Tert-butyl 5-aminohexahydrocyclopenta[c]pyrrole-2(1H)-carboxylate (100 mg, 442 μmol) was added to a solution of TEA (224 mg, 308 μL, 2.21 mmol), HATU (336 mg, 884 μmol) and 5-(trifluoromethyl)furan-2-carboxylic acid (159 mg, 884 μmol) in CH₂Cl₂ (10 mL) at r.t. Then the mixture was stirred at r.t. for 3 h. LC-MS detection indicated that the starting materials were consumed. The mixture was concentrated and the residue was purified by chromatograph on silica gel column to afford tert-butyl 5-[[5-(trifluoromethyl)furan-2-carbonyl]amino]-3,3a,4,5,6,6a-hexahydro-1H-cyclopenta[c]pyrrole-2-carboxylate (120 mg). MS obsd. (ESI⁺) [(M+H)⁺]: 389.2.

### Step 2: Preparation of N-(1,2,3,3a,4,5,6,6a-octahydrocyclopenta[c]pyrrol-5-yl)-5-(trifluoromethyl)furan-2-carboxamide 2,2,2-trifluoroacetic acid salt (123-b)

TFA (352 mg, 3.09 mmol) was added to a solution of tert-butyl 5-(5-(trifluoromethyl)furan-2-carboxamido)hexahydrocyclopenta[c]pyrrole-2(1H)-carboxylate (120 mg, 309 μmol) in CH₂Cl₂ (10 ml) at r.t. Then the mixture was stirred at r.t. for 3 h. LC-MS detection indicated that the starting materials were consumed. The mixture was concentrated to afford crude N-(1,2,3,3a,4,5,6,6a-octahydrocyclopenta[c]pyrrol-5-yl)-5-(trifluoromethyl)furan-2-carboxamide 2,2,2-trifluoroacetic acid salt (130 mg). MS obsd. (ESI⁺) [(M+H)⁺]: 289.2.

### Step 3: Preparation of N-[2-(5-chloro-1,3-benzoxazol-2-yl)-3,3a,4,5,6,6a-hexahydro-1H-cyclopenta[c]pyrrol-5-yl]-5-(trifluoromethyl)furan-2-carboxamide (123)

2,5-Dichlorobenzo[d]oxazole (60.8 mg, 0.32 mmol) was added to a solution of TEA (327 mg, 450 μL, 3.23 mmol) and N-(octahydrocyclopenta[c]pyrrol-5-yl)-5-(trifluoromethyl)furan-2-carboxamide 2,2,2-trifluoroacetate (130 mg, 0.32 mmol) in CH₂Cl₂ (10 mL) at r.t. Then the mixture was stirred at 50° C. for 3 h. LC-MS detection indicated that the reaction was completed. The mixture was concentrated and the residue was purified by prep-HPLC to afford N-[2-(5-chloro-1,3-benzoxazol-2-yl)-3,3a,4,5,6,6a-hexahydro-1H-cyclopenta[c]pyrrol-5-yl]-5-(trifluoromethyl)furan-2-carboxamide (13 mg). MS obsd. (ESI⁺) [(M+H)⁺]: 440.1. ¹H NMR (400 MHz, CD₃OD) δ ppm: 7.34 (d, J=8.6 Hz, 1H), 7.28 (d, J=2.1 Hz, 1H), 7.22 (dd, J=0.7, 3.6 Hz, 1H), 7.14 (dd, J=1.0, 3.7 Hz, 1H), 7.08 (dd, J=2.1, 8.5 Hz, 1H), 4.57 (t, J=7.4 Hz, 1H), 3.91 (dd, J=8.1, 11.1 Hz, 2H), 3.55 (dd, J=3.9, 11.1 Hz, 2H), 3.16-3.08 (m, 2H), 2.08-2.01 (m, 4H).

### Example 124

### N-[2-(5-chloro-1,3-benzoxazol-2-yl)-3,3a,4,5,6,6a-hexahydro-1H-cyclopenta[c]pyrrol-5-yl]-1,1-dioxo-thiolane-3-carboxamide

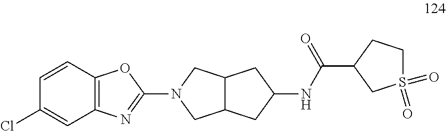 [SMILES: O=C(NC1CC2CN(c3nc4cc(Cl)ccc4o3)CC2C1)C1CCS(=O)(=O)C1]

The title compound was prepared in analogy to the procedure described for the preparation of Example 123, by using 1,1-dioxothiolane-3-carboxylic acid instead of 5-(trifluoromethyl)furan-2-carboxylic acid. MS obsd. (ESI⁺)[(M+H)⁺]: 443.1. ¹H NMR (400 MHz, CD₃OD) δ ppm: 7.33-7.28 (m, 1H), 7.27-7.24 (m, 1H), 7.05 (dd, J=2.0, 8.5 Hz, 1H), 4.27-4.17 (m, 1H), 3.89-3.76 (m, 2H), 3.64 (dd, J=3.1, 10.9 Hz, 2H), 3.53-3.48 (m, 1H), 3.29-3.18 (m, 4H), 3.11-3.02 (m, 1H), 2.92-2.85 (m, 1H), 2.45-2.37 (m, 2H), 2.30-2.21 (m, 1H), 2.09-1.93 (m, 1H), 1.48-1.37 (m, 2H).

### Example 125

### N-[3-(5-chloro-1,3-benzoxazol-2-yl)-3-azabicyclo[3.1.0]hexan-6-yl]-5-(trifluoromethyl)furan-2-carboxamide

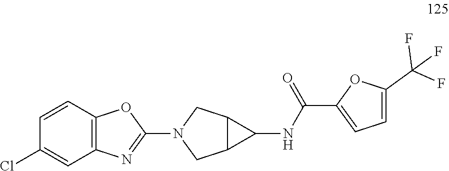 [SMILES: O=C(NC1C2CN(c3nc4cc(Cl)ccc4o3)CC21)c1ccc(C(F)(F)F)o1]

The title compound was prepared in analogy to the procedure described for the preparation of Example 123, by using tert-butyl 6-amino-3-azabicyclo[3.1.0]hexane-3-carboxylate instead of tert-butyl 5-aminohexahydrocyclopenta[c]pyrrole-2(1H)-carboxylate. MS obsd. (ESI⁺) [(M+H)⁺]: 412.1. ¹H NMR (400 MHz, CD₃OD) δ ppm: 7.30 (d, J=8.6 Hz, 1H), 7.25 (d, J=2.1 Hz, 1H), 7.23 (dd, J=0.7, 3.7 Hz, 1H), 7.14 (dd, J=1.1, 3.7 Hz, 1H), 7.04 (dd, J=2.1, 8.5 Hz, 1H), 4.01 (d, J=10.5 Hz, 2H), 3.79 (ddd, J=1.3, 2.4, 10.5 Hz, 2H), 2.66 (t, J=2.4 Hz, 1H), 2.14-2.09 (m, 2H).

### Example 126

### PHH Natural Infection Assay

Detailed procedures regarding primary human hepatocyte (PHH) HBV natural infection assay are described as below. One tube of frozen PHH (10 million cells) is thawed in 37° C. water bath and then transferred to 20 mL of PHH thawing medium (Sigma, InVitroGRO HT Medium, Cat. S03319) with gently mixing. The cells were then centrifuged at 80 g/min for 5 min, the supernatant was discarded and the tube was refilled with 25 mL of PHH plating medium (Sigma, InVitroGRO CP Medium, Cat. S03317). The tube was shaken very gently to re-suspend all cells, and then 50 μL of cells were transferred to each well 384-well collagen I coated plate with appropriate liquid handling equipment, e.g. Integra VIAFLO384 or Agilent Bravo. The cells were then cultured for 24 hours in a cell incubator. For HBV infection, after PHH attachment on the culture plate, the plating medium was removed and replenished with PHH culture medium containing HBV virus. The PHH culture medium was prepared with Dulbecco's Modified Eagle Medium (DMEM)/F12 (1:1 in volume ratio) containing 10% fetal bovine serum (Gibco, Cat. 10099141), 5 ng/mL human epidermal growth factor (Gibco, Cat. PHG0311L), 20 ng/mL dexamethasone (Sigma, Cat. D4902-100 mg), 250 ng/mL human recombinant insulin (Gibco, Cat. 41400045) and 100 U/mL penicillin. HBV virus at 200 genome equivalent (GE) per cell with 4% PEG8000 (Sigma, Cat. P1458) containing culture medium were added to the PHH culture medium for infection. The cells were then cultured for 24 hours in cell incubator. Then the cell culture supernatant was removed. The HBV-infected PHH were cultured with sandwich culture method with MEI culture medium containing 1% DMSO and 0.25 mg/mL matrix gel for 72 hours. The supernatant was then refreshed with PHH culture medium containing different concentrations of testing compounds for two times with 72-hour interval. At the end of treatment, the supernatant was collected for viral markers measurements, including HBsAg, HBeAg, HBV DNA and cytotoxicity. HBsAg and HBeAg were detected using alphalisa method using their specific antibodies. For HBV DNA detection, HBV DNA Quantitative Fluorescence Diagnostic Kit (Sansure Biotech Inc.) was used following the manufacture's protocol. Cytotoxicity was determined using Cell Counting Kit-8 (CCK8, Dojindo Molecular Technologies, Inc.).

The compounds of the present invention were tested for their capacity to inhibit HBsAg and HBeAg as described herein. The Examples were tested in the above assay and found to have IC₅₀ below 10 μM. Results of PHH assay are given in Table 2.

TABLE 2
Activity data of compounds of this invention

| Example No. | HBsAg IC₅₀ (μM) | HBeAg IC₅₀ (μM) | CC₅₀ (μM) |
|---|---|---|---|
| Example 1 | 9.400 | 7.130 | >10.000 |
| Example 2 | 8.095 | 6.313 | >10.000 |
| Example 3 | 7.840 | 6.381 | >10.000 |
| Example 4 | 6.282 | 5.741 | >10.000 |
| Example 5 | 4.682 | 4.932 | >10.000 |
| Example 6 | 4.362 | 4.428 | >10.000 |
| Example 7 | 3.835 | 3.450 | 96.691 |
| Example 8 | 3.137 | 2.854 | >10.000 |
| Example 9 | 1.499 | 1.829 | >10.000 |
| Example 10 | 9.667 | 10.398 | >100.000 |
| Example 11 | 9.139 | 7.846 | >10.000 |
| Example 12 | 4.593 | 5.444 | >100.000 |
| Example 13 | 9.368 | 8.501 | >10.000 |
| Example 14 | 3.823 | 4.164 | >10.000 |
| Example 15 | 3.974 | 3.635 | >10.000 |
| Example 16 | 3.741 | 3.672 | >10.000 |
| Example 17 | 8.229 | >10.000 | >10.000 |
| Example 18 | 3.902 | 3.778 | >10.000 |
| Example 19 | 7.739 | 3.755 | >10.000 |
| Example 20 | 3.879 | 4.053 | >10.000 |
| Example 21 | 7.148 | 5.236 | >100.000 |
| Example 22 | 5.874 | 4.821 | 82.689 |
| Example 23 | 7.633 | 6.625 | >100.000 |
| Example 24 | 7.288 | 8.306 | >100.000 |
| Example 25 | 9.801 | 10.309 | >100.000 |
| Example 26 | 8.763 | 4.647 | >100.000 |
| Example 27 | 5.671 | 3.688 | >100.000 |
| Example 28 | 3.312 | 2.178 | 76.590 |
| Example 29 | 7.133 | 7.373 | >100.000 |
| Example 30 | 3.500 | 2.698 | >100.000 |
| Example 31 | 6.980 | 6.222 | >100.000 |
| Example 32 | 4.250 | 10.306 | >100.000 |
| Example 33 | 4.509 | 4.404 | >100.000 |
| Example 34 | 5.497 | 4.475 | >100.000 |
| Example 35 | 2.364 | 2.220 | >100.000 |
| Example 36 | 8.276 | 4.034 | >100.000 |
| Example 37 | 7.749 | 6.264 | >10.000 |
| Example 38 | 6.890 | 5.732 | >10.000 |
| Example 39 | 6.471 | 4.374 | >100.000 |
| Example 40 | 6.134 | 4.782 | >100.000 |
| Example 41 | 5.867 | 3.806 | >10.000 |
| Example 42 | 5.656 | 4.668 | >100.000 |
| Example 43 | 1.757 | 1.009 | 84.809 |
| Example 44 | 2.267 | 1.282 | >100.000 |
| Example 45 | 2.729 | 1.297 | 80.082 |
| Example 46 | 4.742 | 2.856 | >100.000 |
| Example 47 | 4.239 | 2.307 | >100.000 |
| Example 48 | 3.798 | 2.946 | >100.000 |
| Example 49 | 3.099 | 1.696 | >100.000 |
| Example 50 | 1.657 | 1.488 | 95.444 |
| Example 51 | 2.219 | 1.949 | >100.000 |
| Example 52 | 1.499 | 1.258 | >100.000 |
| Example 53 | 1.406 | 0.785 | 93.232 |
| Example 54 | 1.271 | 0.828 | 84.463 |
| Example 55 | 1.225 | 0.966 | 71.054 |
| Example 56 | 1.222 | 0.996 | >100.000 |
| Example 57 | 0.996 | 0.953 | >100.000 |
| Example 58 | 0.918 | 0.812 | >100.000 |
| Example 59 | 2.840 | 1.459 | >10.000 |
| Example 60 | 2.517 | 2.550 | >10.000 |
| Example 61 | 0.189 | 0.402 | 96.048 |
| Example 62 | 0.449 | 0.393 | 77.365 |
| Example 63 | 2.471 | 1.419 | >100.000 |
| Example 64 | 2.516 | 1.540 | >100.000 |
| Example 65 | 0.806 | 1.071 | 92.308 |
| Example 66 | 0.781 | 0.608 | 84.248 |
| Example 67 | 1.769 | 2.134 | >100.000 |
| Example 68 | 4.350 | 4.685 | 86.939 |
| Example 69 | 4.409 | 4.010 | *83.201 |
| Example 70 | 6.089 | 2.962 | >100.000 |
| Example 71 | 8.212 | 5.959 | 94.127 |
| Example 72 | 7.899 | 4.355 | 81.424 |
| Example 73 | 6.322 | 3.051 | >100.000 |
| Example 74 | 4.332 | 3.735 | >100.000 |
| Example 75 | 8.284 | 7.814 | 91.172 |
| Example 76 | 2.263 | 2.269 | >100.000 |
| Example 77 | 5.045 | 2.980 | >100.000 |
| Example 78 | 1.466 | 1.197 | 57.940 |
| Example 79 | 8.839 | 11.555 | >100.000 |
| Example 80 | 2.704 | 2.792 | >100.000 |
| Example 81 | 3.494 | 2.278 | 67.277 |
| Example 82 | 3.086 | 2.303 | 76.284 |
| Example 83 | 2.486 | 1.792 | 98.603 |
| Example 84 | 2.021 | 1.368 | 58.590 |
| Example 85 | 8.751 | 6.131 | 70.912 |
| Example 86 | 2.892 | 2.798 | 82.679 |
| Example 87 | 1.427 | 1.132 | >100.000 |
| Example 88 | 1.519 | 1.248 | 28.865 |
| Example 89 | 6.758 | 3.927 | 88.102 |
| Example 90 | 1.346 | 1.082 | 73.872 |
| Example 91 | 1.118 | 0.676 | 89.198 |
| Example 92 | 0.857 | 0.750 | 83.899 |
| Example 93 | 1.816 | 1.190 | >100.000 |
| Example 94 | 1.730 | 1.396 | 90.573 |
| Example 95 | 1.672 | 3.624 | >100.000 |
| Example 96 | 0.957 | 0.727 | 93.570 |
| Example 97 | 3.520 | 2.480 | >100.000 |
| Example 98 | 4.232 | 2.231 | >100.000 |
| Example 99 | 2.634 | 2.019 | 94.074 |
| Example 100 | 2.440 | 1.719 | >100.000 |
| Example 101 | 4.161 | 4.294 | >100.000 |
| Example 102 | 3.599 | 3.765 | >100.000 |
| Example 103 | 1.353 | 1.848 | 72.731 |
| Example 104 | 1.163 | 0.996 | 94.771 |
| Example 105 | 1.116 | 1.029 | 58.953 |
| Example 106 | 0.590 | 0.480 | >100.000 |
| Example 107 | 0.496 | 0.427 | >100.000 |
| Example 108 | 6.693 | 4.176 | >10.000 |
| Example 109 | 5.878 | 4.657 | >100.000 |
| Example 110 | 5.839 | 5.583 | >10.000 |
| Example 111 | 5.446 | 3.469 | 85.663 |
| Example 112 | 3.684 | 4.239 | >10.000 |
| Example 113 | 3.594 | 3.266 | >100.000 |
| Example 114 | 3.542 | 3.100 | 93.312 |
| Example 115 | 2.916 | 2.637 | >100.000 |
| Example 116 | 2.858 | 2.337 | 68.500 |
| Example 117 | 2.373 | 2.910 | >100.000 |
| Example 118 | 2.368 | 2.357 | >10.000 |
| Example 119 | 1.916 | 1.364 | 85.778 |
| Example 120 | 1.550 | 1.427 | 66.595 |
| Example 121 | 1.537 | 1.157 | >100.000 |
| Example 122 | 0.384 | 0.473 | >100.000 |
| Example 123 | 4.811 | 5.468 | >100.000 |
| Example 124 | 6.591 | 12.954 | >100.000 |
| Example 125 | 4.742 | 5.407 | >100.000 |

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

It is to be understood that the invention is not limited to the particular embodiments and aspects of the disclosure described above, as variations of the particular embodiments and aspects may be made and still fall within the scope of the appended claims. All documents cited to or relied upon herein are expressly incorporated by reference.

We claim:

1. A compound of the formula (I), 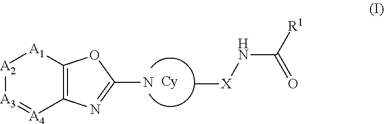
wherein
R¹ is C₁₋₆alkyl, C₁₋₆alkylamino, (C₁₋₆alkyl)₂amino, C₁₋₆alkoxy, C₃₋₇cycloalkyl, phenyl, heterocyclyl or hetercyclylC₁₋₆alkyl; wherein C₁₋₆alkyl, C₁₋₆alkylamino, (C₁₋₆alkyl)₂amino, C₁₋₆alkoxy, C₃₋₇cycloalkyl, phenyl, heterocyclyl and hetercyclylC₁₋₆alkyl are unsubstituted or substituted by one or two or three substituents independently selected from aminocarbonyl, haloC₁₋₆alkyl, C₃₋₇cycloalkylsulfonyl, C₁₋₆alkylsulfonyl, aminosulfonyl, C₁₋₆alkylphenylsulfonyl, OH, halogen, C₁₋₆alkyl, C₁₋₆alkoxy, C₁₋₆alkoxyC₁₋₆alkoxy, C₁₋₆alkylsulfonimidoyl, C₁₋₆alkylsulfonylC₁₋₆alkyl, C₃₋₇cycloalkylC₁₋₆alkylsulfonimidoyl, C₃₋₇cycloalkylaminosulfonyl, haloC₃₋₇cycloalkylaminosulfonyl, C₁₋₆alkylsulfinyl, C₃₋₇cycloalkylC₁₋₆alkylsulfonyl, C₃₋₇cycloalkylC₁₋₆alkylsulfinyl, C₃₋₇cycloalkylcarbonylaminosulfonyl and C₃₋₇cycloalkylsulfonimidoyl;
Cy is N-containing heterocyclyl; wherein N-containing heterocyclyl is unsubstituted or substituted by C₁₋₆alkyl;
X is a bond or CH₂;
A₁ is N or CR²; wherein R² is H, halogen, C₁₋₆alkyl, or C₁₋₆alkoxy;
A₂ is N or CR³; wherein R³ is H, halogen, C₁₋₆alkyl, or C₁₋₆alkoxy;
A₃ is N or CR⁴; wherein R⁴ is H, halogen, C₁₋₆alkyl, or C₁₋₆alkoxy;
A₄ is N or CR⁵; wherein R⁵ is H, halogen, C₁₋₆alkyl, or C₁₋₆alkoxy;
or a pharmaceutically acceptable salt thereof.

2. A compound according to claim 1, wherein
R¹ is C₁₋₆alkyl, C₁₋₆alkylamino, (C₁₋₆alkyl)₂amino, C₁₋₆alkoxy, C₃₋₇cycloalkyl, phenyl, furyl, pyridyl, pyrrolidinyl, piperidyl, oxetanyl, tetrahydrofuranyl, tetrahydrothiopyranyl, azetidinylmethyl, pyrrolidinylmethyl, piperidylmethyl, tetrahydrofuranylmethyl, 1-oxothianyl, 1,1-dioxothietanyl, 1,1-dioxothiolanyl, 1,1-dioxothianyl, 1,1-dioxothietanylmethyl or 2,2-dioxo-2-λ⁶-thiaspiro[3.3]heptanyl; wherein C₁₋₆alkyl, C₃₋₇cycloalkyl, phenyl, furyl, pyridyl, pyrrolidinyl, piperidyl, tetrahydrothiopyranyl, azetidinylmethyl, pyrrolidinylmethyl, piperidylmethyl, 1-oxothianyl, 1,1-dioxothiolanyl and 1,1-dioxothianyl are unsubstituted or substituted by one or two or three substituents independently selected from aminocarbonyl, haloC₁₋₆alkyl, C₃₋₇cycloalkylsulfonyl, C₁₋₆alkylsulfonyl, aminosulfonyl, C₁₋₆alkylphenylsulfonyl, OH, halogen, C₁₋₆alkyl, C₁₋₆alkoxy, C₁₋₆alkoxyC₁₋₆alkoxy, C₁₋₆alkylsulfonimidoyl, C₁₋₆alkylsulfonylC₁₋₆alkyl, C₃₋₇cycloalkylC₁₋₆alkylsulfonimidoyl, C₃₋₇cycloalkylaminosulfonyl, haloC₃₋₇cycloalkylaminosulfonyl, C₁₋₆alkylsulfinyl, C₃₋₇cycloalkylC₁₋₆alkylsulfonyl, C₃₋₇cycloalkylC₁₋₆alkylsulfinyl, C₃₋₇cycloalkylcarbonylaminosulfonyl and C₃₋₇cycloalkylsulfonimidoyl;
Cy is selected from the group consisting of 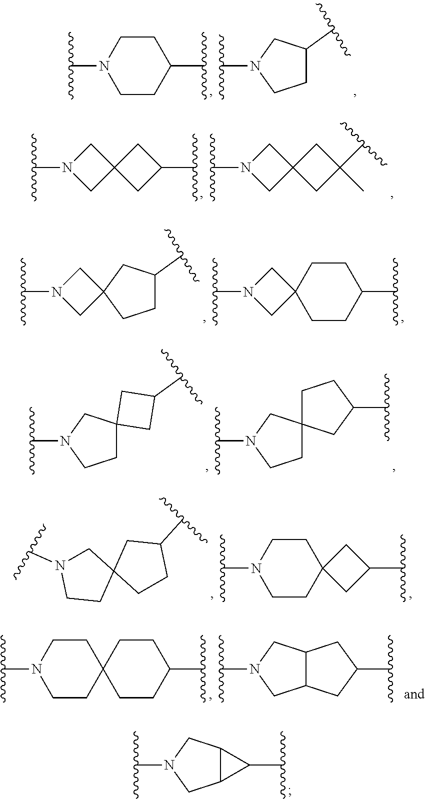
X is a bond or CH₂;
A₁ is N or CH;
A₂ is CR³; wherein R³ is H or halogen;
A₃ is CR⁴; wherein R⁴ is H or halogen;
A₄ is CH;
or a pharmaceutically acceptable salt thereof.

3. A compound according to claim 2 wherein
R¹ is methyl, ethyl, propyl, isopropyl, tert-butyl, isobutyl, dimethylamino, ethylamino, ethoxy, isobutoxy, cyclopropyl, cyclobutyl, cyclopentyl, cyclohextyl, phenyl, furyl, pyridyl, pyrrolidinyl, piperidyl, oxetanyl, tetrahydrofuranyl, tetrahydrothiopyranyl, azetidinylmethyl, pyrrolidinylmethyl, piperidylmethyl, tetrahydrofuranylmethyl, 1-oxothianyl, 1,1-dioxothietanyl, 1,1-dioxothiolanyl, 1,1-dioxothianyl, 1,1-dioxothietanylmethyl or 2,2-dioxo-2-λ⁶-thiaspiro[3.3]heptanyl; wherein methyl, ethyl, propyl, isopropyl, cyclopropyl, cyclobutyl, cyclopentyl, cyclohextyl, phenyl, furyl, pyridyl, pyrrolidinyl, piperidyl, tetrahydrothiopyranyl, azetidinylmethyl, pyrrolidinylmethyl, piperidylmethyl, 1-oxothianyl, 1,1-dioxothiolanyl and 1,1-dioxothianyl are unsubstituted or substituted by one or two or three substituents independently selected from aminocarbonyl, CF₃, cyclopropylsulfonyl, methylsulfonyl, aminosulfonyl, methylphenylsulfonyl, OH, F, methyl, methoxy, methoxyethoxy, methylsulfonimidoyl, isopropoxy, ethoxy, isobutylsulfonimidoyl, methylsulfonylmethyl, ethylsulfonyl, isobutylsulfonyl, cyclopropylmethylsulfonimidoyl, cyclopropylaminosulfonyl, difluorocyclobutylaminosulfonyl, cyclopropylmethylsulfinyl, isobutylsulfinyl, methylsulfinyl, cyclopropylmethylsulfonyl, ethylsulfinyl, cyclobutylcarbonylaminosulfonyl, ethylsulfonimidoyl and cyclopropylsulfonimidoyl;
Cy is selected from the group consisting of 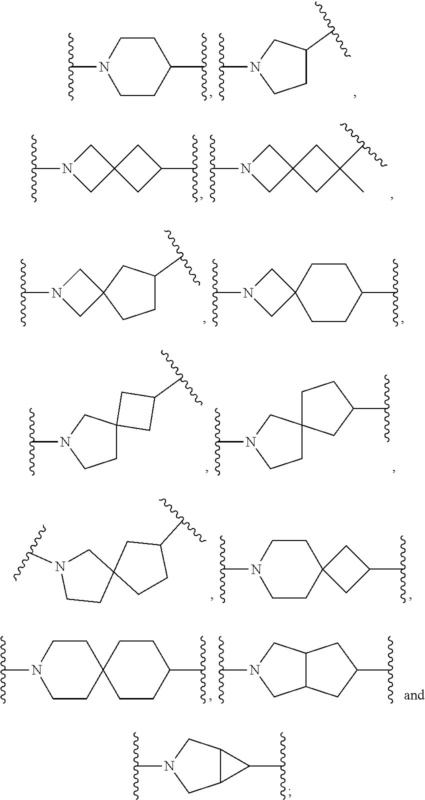
X is a bond or CH₂;
A₁ is N or CH;
A₂ is CR³; wherein R³ is H or Cl;
A₃ is CR⁴; wherein R⁴ is H or Cl;
A₄ is CH;
or a pharmaceutically acceptable salt thereof.

4. A compound according to claim 1, or a pharmaceutically acceptable salt thereof, wherein A₁ is CH.

5. A compound according to claim 1, or a pharmaceutically acceptable salt thereof, wherein A₁, A₂, A₃ and A₄ are not CH simultaneously.

6. A compound according to claim 1, or a pharmaceutically acceptable salt thereof, wherein Cy is selected from the group consisting of

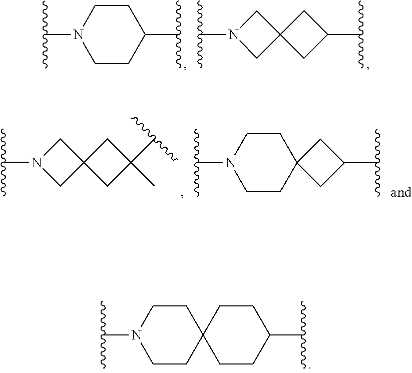

7. A compound according to claim 6, or a pharmaceutically acceptable salt thereof, wherein when Cy is 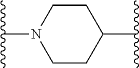 [SMILES: *C1CCN(*)CC1]
and X is CH₂.

8. A compound according to claim 6, or a pharmaceutically acceptable salt thereof, wherein when Cy is 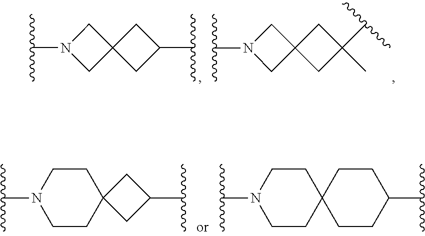
and X is a bond.

9. A compound according to claim 1, or a pharmaceutically acceptable salt thereof, wherein R¹ is C₁₋₆alkyl, furyl, pyridyl, tetrahydrofuranyl, tetrahydrothiopyranyl, tetrahydrofuranylmethyl, 1,1-dioxothietanyl, 1,1-dioxothiolanyl, 1,1-dioxothianyl or 2,2-dioxo-2-λ⁶-thiaspiro[3.3]heptanyl; wherein C₁₋₆alkyl, furyl, pyridyl, tetrahydrothiopyranyl and 1,1-dioxothiolanyl are unsubstituted or substituted one time independently selected from haloC₁₋₆alkyl, C₃₋₇cycloalkylsulfonyl, C₁₋₆alkylsulfonyl, C₁₋₆alkyl, C₁₋₆alkoxy, C₁₋₆alkylsulfonimidoyl, C₃₋₇cycloalkylC₁₋₆alkylsulfonimidoyl, C₁₋₆alkylsulfinyl, C₃₋₇cycloalkylC₁₋₆alkylsulfonyl, C₃₋₇cycloalkylC₁₋₆alkylsulfinyl, and C₃₋₇cycloalkylsulfonimidoyl.

10. A compound according to claim 9, or a pharmaceutically acceptable salt thereof, wherein R¹ is ethyl, tert-butyl, furyl, pyridyl, tetrahydrofuranyl, tetrahydrothiopyranyl, tetrahydrofuranylmethyl, 1,1-dioxothietanyl, 1,1-dioxothiolanyl, 1,1-dioxothianyl or 2,2-dioxo-2-λ⁶-thiaspiro[3.3]heptanyl; wherein ethyl, furyl, pyridyl, tetrahydrothiopyranyl and 1,1-dioxothiolanyl are unsubstituted or substituted one time independently selected from methylsulfonyl, methoxy, isobutylsulfonimidoyl, ethylsulfonyl, isobutylsulfonyl, CF₃, cyclopropylmethylsulfonimidoyl, cyclopropylmethylsulfinyl, isobutylsulfinyl, cyclopropylmethylsulfonyl, cyclopropylsulfonyl, methyl, ethylsulfinyl and cyclopropylsulfonimidoyl.

11. A compound according to claim 1 wherein
R¹ is C₁₋₆alkyl, furyl, pyridyl, tetrahydrofuranyl, tetrahydrothiopyranyl, tetrahydrofuranylmethyl, 1,1-dioxothietanyl, 1,1-dioxothiolanyl, 1,1-dioxothianyl or 2,2-dioxo-2-λ⁶-thiaspiro[3.3]heptanyl; wherein C₁₋₆alkyl, furyl, pyridyl, tetrahydrothiopyranyl and 1,1-dioxothiolanyl are unsubstituted or substituted one time independently selected from haloC₁₋₆alkyl, C₃₋₇cycloalkylsulfonyl, C₁₋₆alkylsulfonyl, C₁₋₆alkyl, C₁₋₆alkoxy, C₁₋₆ alkylsulfonimidoyl, C₃₋₇ cycloalkylC₁₋₆ alkylsulfonimidoyl, C₁₋₆ alkylsulfinyl, C₃₋₇cycloalkylC₁₋₆alkylsulfonyl, C₃₋₇cycloalkylC₁₋₆alkylsulfinyl, and C₃₋₇cycloalkylsulfonimidoyl;
Cy is selected from the group consisting of 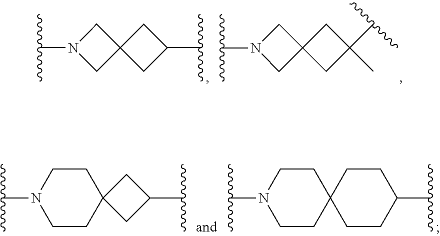
X is a bond;
A₁ is CH;
A₂ is CR³; wherein R³ is H or halogen;
A₃ is CR⁴; wherein R⁴ is H or halogen;
A₄ is CH;
wherein with the proviso that A₁, A₂, A₃ and A₄ are not CH simultaneously;
or a pharmaceutically acceptable salt thereof.

12. A compound according to claim 11, wherein
R¹ is ethyl, tert-butyl, furyl, pyridyl, tetrahydrofuranyl, tetrahydrothiopyranyl, tetrahydrofuranylmethyl, 1,1-dioxothietanyl, 1,1-dioxothiolanyl, 1,1-dioxothianyl or 2,2-dioxo-2-λ⁶-thiaspiro[3.3]heptanyl; wherein ethyl, furyl, pyridyl, tetrahydrothiopyranyl and 1,1-dioxothiolanyl are unsubstituted or substituted one time independently selected from methylsulfonyl, methoxy, isobutylsulfonimidoyl, ethylsulfonyl, isobutylsulfonyl, CF₃, cyclopropylmethylsulfonimidoyl, cyclopropylmethylsulfinyl, isobutylsulfinyl, cyclopropylmethylsulfonyl, cyclopropylsulfonyl, methyl, ethylsulfinyl and cyclopropylsulfonimidoyl;
Cy is selected from the group consisting of 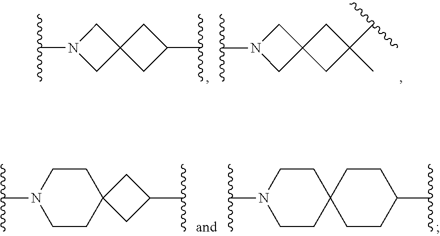
X is a bond;
A₁ is CH;
A₂ is CR³; wherein R³ is H or Cl;
A₃ is CR⁴; wherein R⁴ is H or Cl;
A₄ is CH;
wherein with the proviso that A₁, A₂, A₃ and A₄ are not CH simultaneously;
or a pharmaceutically acceptable salt thereof.

13. A compound according to claim 1, selected from the group consisting of:
N2-[[1-(5-chloro-1,3-benzoxazol-2-yl)-4-piperidyl]methyl]furan-2,5-dicarboxamide;
N-[[1-(5-chloro-1,3-benzoxazol-2-yl)-4-piperidyl]methyl]-6-(trifluoromethyl)pyridine-3-carboxamide;
N-[[1-(5-chloro-1,3-benzoxazol-2-yl)-4-piperidyl]methyl]-2-cyclopropylsulfonyl-pyridine-4-carboxamide;
N-[[1-(5-chloro-1,3-benzoxazol-2-yl)-4-piperidyl]methyl]-3-methylsulfonyl-benzamide;
N-[[1-(5-chloro-1,3-benzoxazol-2-yl)-4-piperidyl]methyl]-2-methylsulfonyl-pyridine-4-carboxamide;
N4-[[1-(5-chloro-1,3-benzoxazol-2-yl)-4-piperidyl]methyl]pyridine-2,4-dicarboxamide;
N-[[1-(5-chloro-1,3-benzoxazol-2-yl)-4-piperidyl]methyl]-5-(trifluoromethyl)furan-2-carboxamide;
N-[[1-(5-chloro-1,3-benzoxazol-2-yl)-4-piperidyl]methyl]-5-sulfamoyl-furan-2-carboxamide;
N-[[1-(5-chloro-1,3-benzoxazol-2-yl)-4-piperidyl]methyl]-5-methylsulfonyl-pyridine-3-carboxamide;
N-[[1-(5-chloro-1,3-benzoxazol-2-yl)-4-piperidyl]methyl]-1,1-dioxo-thietane-3-carboxamide;
N-[[1-(5-chloro-1,3-benzoxazol-2-yl)pyrrolidin-3-yl]methyl]-2-cyclopropylsulfonyl-pyridine-4-carboxamide;
N-[1-(5-chloro-1,3-benzoxazol-2-yl)-4-piperidyl]-5-(trifluoromethyl)furan-2-carboxamide;
N-[[1-(5-chloro-1,3-benzoxazol-2-yl)-4-piperidyl]methyl]-1-methylsulfonyl-pyrrolidine-3-carboxamide;
N-[[1-(5-chloro-1,3-benzoxazol-2-yl)-4-piperidyl]methyl]-1-(p-tolylsulfonyl)pyrrolidine-3-carboxamide;
N-[[1-(5-chloro-1,3-benzoxazol-2-yl)-4-piperidyl]methyl]-1-methylsulfonyl-piperidine-4-carboxamide;
N-[[1-(5-chloro-1,3-benzoxazol-2-yl)-4-piperidyl]methyl]-1-(p-tolylsulfonyl)piperidine-4-carboxamide;
N-[[1-(5-chloro-1,3-benzoxazol-2-yl)-4-piperidyl]methyl]-2-(1-methylsulfonylazetidin-3-yl)acetamide;
N-[[1-(5-chloro-1,3-benzoxazol-2-yl)-4-piperidyl]methyl]-2-[1-(p-tolylsulfonyl)pyrrolidin-3-yl]acetamide;
N-[[1-(5-chloro-1,3-benzoxazol-2-yl)-4-piperidyl]methyl]-2-(1-methylsulfonyl-4-piperidyl)acetamide;
N-[[1-(5-chloro-1,3-benzoxazol-2-yl)-4-piperidyl]methyl]-2-[1-(p-tolylsulfonyl)-4-piperidyl]acetamide;
ethyl N-[2-(5-chloro-1,3-benzoxazol-2-yl)-2-azaspiro[3.3]heptan-6-yl]carbamate;
isobutyl N-[2-(5-chloro-1,3-benzoxazol-2-yl)-2-azaspiro[3.3]heptan-6-yl]carbamate;
3-[2-(5-chloro-1,3-benzoxazol-2-yl)-2-azaspiro[3.3]heptan-6-yl]-1,1-dimethyl-urea;
1-[2-(5-chloro-1,3-benzoxazol-2-yl)-2-azaspiro[3.3]heptan-6-yl]-3-ethyl-urea;
N-[2-(5-chloro-1,3-benzoxazol-2-yl)-2-azaspiro[3.3]heptan-6-yl]-4,4,4-trifluoro-butanamide;
N-[2-(5-chloro-1,3-benzoxazol-2-yl)-2-azaspiro[3.3]heptan-6-yl]-1,1-dioxo-thiolane-3-carboxamide;
N-[2-(5-chloro-1,3-benzoxazol-2-yl)-2-azaspiro[3.3]heptan-6-yl]-2,2-dimethyl-propanamide;
N-[2-(5-chloro-1,3-benzoxazol-2-yl)-2-azaspiro[3.3]heptan-6-yl]-3-methyl-butanamide;
N-[2-(5-chloro-1,3-benzoxazol-2-yl)-2-azaspiro[3.3]heptan-6-yl]-2-hydroxy-2-methyl-propanamide;
N-[2-(5-chloro-1,3-benzoxazol-2-yl)-2-azaspiro[3.3]heptan-6-yl]-2,2-difluoro-cyclopropanecarboxamide;
N-[2-(5-chloro-1,3-benzoxazol-2-yl)-2-azaspiro[3.3]heptan-6-yl]-3,3-difluoro-cyclobutanecarboxamide;
N-[2-(5-chloro-1,3-benzoxazol-2-yl)-2-azaspiro[3.3]heptan-6-yl]-3,3-difluoro-cyclopentanecarboxamide;
N-[2-(5-chloro-1,3-benzoxazol-2-yl)-2-azaspiro[3.3]heptan-6-yl]-3-methyl-1,1-dioxo-thiolane-3-carboxamide;
N-[2-(5-chloro-1,3-benzoxazol-2-yl)-2-azaspiro[3.3]heptan-6-yl]-1,1-dioxo-thietane-3-carboxamide;
N-[2-(5-chloro-1,3-benzoxazol-2-yl)-2-azaspiro[3.3]heptan-6-yl]-2-(1,1-dioxothietan-3-yl)acetamide;
N-[2-(5-chloro-1,3-benzoxazol-2-yl)-2-azaspiro[3.3]heptan-6-yl]-1,1-dioxo-thiane-4-carboxamide;
N-[2-(6-chloro-1,3-benzoxazol-2-yl)-2-azaspiro[3.3]heptan-6-yl]-2-cyclopropylsulfonyl-pyridine-4-carboxamide;
N-[2-(6-chloro-1,3-benzoxazol-2-yl)-2-azaspiro[3.3]heptan-6-yl]-2-methoxy-pyridine-4-carboxamide;
N-[2-(5-chloro-1,3-benzoxazol-2-yl)-2-azaspiro[3.3]heptan-6-yl]-2-(2-methoxyethoxy)pyridine-4-carboxamide;
N-[2-(5-chloro-1,3-benzoxazol-2-yl)-2-azaspiro[3.3]heptan-6-yl]-5-(methylsulfonimidoyl)furan-2-carboxamide;
N-[2-(6-chloro-1,3-benzoxazol-2-yl)-2-azaspiro[3.3]heptan-6-yl]-2-isopropoxy-pyridine-4-carboxamide;
N-[2-(5-chloro-1,3-benzoxazol-2-yl)-2-azaspiro[3.3]heptan-6-yl]-5-(trifluoromethyl)furan-2-carboxamide;
N-[2-(5-chloro-1,3-benzoxazol-2-yl)-2-azaspiro[3.3]heptan-6-yl]-2-methoxy-pyridine-4-carboxamide;
N-[2-(5-chloro-1,3-benzoxazol-2-yl)-2-azaspiro[3.3]heptan-6-yl]-5-cyclopropylsulfonyl-furan-2-carboxamide;
N-[2-(5-chloro-1,3-benzoxazol-2-yl)-2-azaspiro[3.3]heptan-6-yl]-2-methyl-pyridine-4-carboxamide;
N-[2-(5-chloro-1,3-benzoxazol-2-yl)-2-azaspiro[3.3]heptan-6-yl]-6-methoxy-pyridine-3-carboxamide;
N-[2-(5-chloro-1,3-benzoxazol-2-yl)-2-azaspiro[3.3]heptan-6-yl]-5-[(S)-methylsulfonimidoyl]furan-2-carboxamide;
N-[2-(6-chlorooxazolo[5,4-b]pyridin-2-yl)-2-azaspiro[3.3]heptan-6-yl]-5-methylsulfonyl-furan-2-carboxamide;
N-[2-(5-chloro-1,3-benzoxazol-2-yl)-2-azaspiro[3.3]heptan-6-yl]-2-ethoxy-pyridine-4-carboxamide;
N-[2-(5-chloro-1,3-benzoxazol-2-yl)-2-azaspiro[3.3]heptan-6-yl]-5-(isobutylsulfonimidoyl)furan-2-carboxamide;
N-[2-(5-chloro-1,3-benzoxazol-2-yl)-2-azaspiro[3.3]heptan-6-yl]-5-(methylsulfonylmethyl)furan-2-carboxamide;
N-[2-(5-chloro-1,3-benzoxazol-2-yl)-2-azaspiro[3.3]heptan-6-yl]-2-methylsulfonyl-pyridine-4-carboxamide;
N-[2-(5-chloro-1,3-benzoxazol-2-yl)-2-azaspiro[3.3]heptan-6-yl]-2-ethylsulfonyl-pyridine-4-carboxamide;
N-[2-(5-chloro-1,3-benzoxazol-2-yl)-2-azaspiro[3.3]heptan-6-yl]-5-isobutylsulfonyl-furan-2-carboxamide;
N-[2-(5-chloro-1,3-benzoxazol-2-yl)-2-azaspiro[3.3]heptan-6-yl]-2-(trifluoromethyl)pyridine-4-carboxamide;
N-[2-(5-chloro-1,3-benzoxazol-2-yl)-2-azaspiro[3.3]heptan-6-yl]-5-methylsulfonyl-furan-2-carboxamide;
N-[2-(5-chloro-1,3-benzoxazol-2-yl)-2-azaspiro[3.3]heptan-6-yl]-5-(cyclopropylmethylsulfonimidoyl)furan-2-carboxamide;
N-[2-(5-chloro-1,3-benzoxazol-2-yl)-2-azaspiro[3.3]heptan-6-yl]-5-ethylsulfonyl-furan-2-carboxamide;
N-[2-(5-chloro-1,3-benzoxazol-2-yl)-2-azaspiro[3.3]heptan-6-yl]-5-(cyclopropylsulfamoyl)furan-2-carboxamide;
N-[2-(5-chloro-1,3-benzoxazol-2-yl)-2-azaspiro[3.3]heptan-6-yl]-5-[(3,3-difluorocyclobutyl)sulfamoyl]furan-2-carboxamide;
N-[2-(5-chloro-1,3-benzoxazol-2-yl)-2-azaspiro[3.3]heptan-6-yl]-5-(cyclopropylmethylsulfinyl)furan-2-carboxamide;
N-[2-(5-chloro-1,3-benzoxazol-2-yl)-2-azaspiro[3.3]heptan-6-yl]-5-isobutylsulfinyl-furan-2-carboxamide;
N-[2-(5-chloro-1,3-benzoxazol-2-yl)-2-azaspiro[3.3]heptan-6-yl]-5-[(R)-methylsulfonimidoyl]furan-2-carboxamide;
N-[2-(5-chloro-1,3-benzoxazol-2-yl)-2-azaspiro[3.3]heptan-6-yl]-5-methylsulfinyl-furan-2-carboxamide;
N-[2-(5-chloro-1,3-benzoxazol-2-yl)-2-azaspiro[3.3]heptan-6-yl]-5-(cyclopropylmethylsulfonyl)furan-2-carboxamide;
N-[2-(5-chloro-1,3-benzoxazol-2-yl)-2-azaspiro[3.3]heptan-6-yl]-2-cyclopropylsulfonyl-pyridine-4-carboxamide;
N-[2-(5-chloro-1,3-benzoxazol-2-yl)-6-methyl-2-azaspiro[3.3]heptan-6-yl]-5-methylsulfonyl-furan-2-carboxamide;
N-[2-(5-chloro-1,3-benzoxazol-2-yl)-6-methyl-2-azaspiro[3.3]heptan-6-yl]-2-(trifluoromethyl)pyridine-4-carboxamide;
N-[2-(5-chloro-1,3-benzoxazol-2-yl)-2-azaspiro[3.4]octan-6-yl]-5-(trifluoromethyl)furan-2-carboxamide;
N-[2-(5-chloro-1,3-benzoxazol-2-yl)-2-azaspiro[3.5]nonan-7-yl]-5-(trifluoromethyl)furan-2-carboxamide;
N-[6-(5-chloro-1,3-benzoxazol-2-yl)-6-azaspiro[3.4]octan-2-yl]-1,1-dioxo-thiolane-3-carboxamide;
N-[6-(5-chloro-1,3-benzoxazol-2-yl)-6-azaspiro[3.4]octan-2-yl]-5-(trifluoromethyl)furan-2-carboxamide;
N-[6-(6-chloro-1,3-benzoxazol-2-yl)-6-azaspiro[3.4]octan-2-yl]-1,1-dioxo-thiolane-3-carboxamide;
N-[2-(5-chloro-1,3-benzoxazol-2-yl)-2-azaspiro[4.4]nonan-8-yl]-1,1-dioxo-thiolane-3-carboxamide;
N-[2-(5-chloro-1,3-benzoxazol-2-yl)-2-azaspiro[4.4]nonan-8-yl]-5-(trifluoromethyl)furan-2-carboxamide;
N-[7-(5-chloro-1,3-benzoxazol-2-yl)-7-azaspiro[3.5]nonan-2-yl]-1,1-dioxo-thiolane-3-carboxamide;
N-[7-(5-chloro-1,3-benzoxazol-2-yl)-7-azaspiro[3.5]nonan-2-yl]-5-(trifluoromethyl)furan-2-carboxamide;
N-[3-(5-chloro-1,3-benzoxazol-2-yl)-3-azaspiro[5.5]undecan-9-yl]-2,2-dimethyl-propanamide;
N-[3-(5-chloro-1,3-benzoxazol-2-yl)-3-azaspiro[5.5]undecan-9-yl]-2-methyl-propanamide;
N-[3-(5-chloro-1,3-benzoxazol-2-yl)-3-azaspiro[5.5]undecan-9-yl]-3-methyl-butanamide;
N-[3-(5-chloro-1,3-benzoxazol-2-yl)-3-azaspiro[5.5]undecan-9-yl]-2-methylsulfonyl-acetamide;
N-[3-(5-chloro-1,3-benzoxazol-2-yl)-3-azaspiro[5.5]undecan-9-yl]-3-sulfamoyl-propanamide;
N-[3-(5-chloro-1,3-benzoxazol-2-yl)-3-azaspiro[5.5]undecan-9-yl]-3-methylsulfonyl-propanamide;
N-[3-(5-chloro-1,3-benzoxazol-2-yl)-3-azaspiro[5.5]undecan-9-yl]-2-tetrahydrofuran-3-yl-acetamide;
N-[3-(5-chloro-1,3-benzoxazol-2-yl)-3-azaspiro[5.5]undecan-9-yl]oxetane-3-carboxamide;
N-[3-(5-chloro-1,3-benzoxazol-2-yl)-3-azaspiro[5.5]undecan-9-yl]tetrahydrofuran-3-carboxamide;
N-[3-(5-chloro-1,3-benzoxazol-2-yl)-3-azaspiro[5.5]undecan-9-yl]-4-methyl-tetrahydrothiopyran-4-carboxamide;
N-[3-(5-chloro-1,3-benzoxazol-2-yl)-3-azaspiro[5.5]undecan-9-yl]-1,1-dioxo-thietane-3-carboxamide;
N-[3-(6-chloro-1,3-benzoxazol-2-yl)-3-azaspiro[5.5]undecan-9-yl]-1,1-dioxo-thietane-3-carboxamide;
N-[3-(5-chloro-1,3-benzoxazol-2-yl)-3-azaspiro[5.5]undecan-9-yl]-1,1-dioxo-thiolane-3-carboxamide;
(3S)—N-[3-(5-chloro-1,3-benzoxazol-2-yl)-3-azaspiro[5.5]undecan-9-yl]-1,1-dioxo-thiolane-3-carboxamide;
(3R)—N-[3-(5-chloro-1,3-benzoxazol-2-yl)-3-azaspiro[5.5]undecan-9-yl]-1,1-dioxo-thiolane-3-carboxamide;
N-[3-(5-chloro-1,3-benzoxazol-2-yl)-3-azaspiro[5.5]undecan-9-yl]-3-methyl-1,1-dioxo-thiolane-3-carboxamide;
N-[3-(6-chloro-1,3-benzoxazol-2-yl)-3-azaspiro[5.5]undecan-9-yl]-1,1-dioxo-thiolane-3-carboxamide;
N-[3-(5-chloro-1,3-benzoxazol-2-yl)-3-azaspiro[5.5]undecan-9-yl]-2,2-dioxo-2-λ⁶-thiaspiro[3.3]heptane-6-carboxamide;
N-[3-(5-chloro-1,3-benzoxazol-2-yl)-3-azaspiro[5.5]undecan-9-yl]-1,1-dioxo-thiane-4-carboxamide;
N-[3-(6-chloro-1,3-benzoxazol-2-yl)-3-azaspiro[5.5]undecan-9-yl]-1,1-dioxo-thiane-4-carboxamide;
N-[3-(5-chloro-1,3-benzoxazol-2-yl)-3-azaspiro[5.5]undecan-9-yl]-4-methyl-1-oxo-thiane-4-carboxamide;
N-[3-(5-chloro-1,3-benzoxazol-2-yl)-3-azaspiro[5.5]undecan-9-yl]-4-methyl-1,1-dioxo-thiane-4-carboxamide;
N-[3-(5-chloro-1,3-benzoxazol-2-yl)-3-azaspiro[5.5]undecan-9-yl]-1,1-dioxo-thiane-3-carboxamide;
N-[3-(5-chloro-1,3-benzoxazol-2-yl)-3-azaspiro[5.5]undecan-9-yl]-3,3-difluoro-cy clopentanecarboxamide;
N-[3-(5-chloro-1,3-benzoxazol-2-yl)-3-azaspiro[5.5]undecan-9-yl]-4,4-difluoro-cyclohexanecarboxamide;
N-[3-(5-chloro-1,3-benzoxazol-2-yl)-3-azaspiro[5.5]undecan-9-yl]-5-ethylsulfinyl-furan-2-carboxamide;
N-[3-(5-chloro-1,3-benzoxazol-2-yl)-3-azaspiro[5.5]undecan-9-yl]-5-(cyclopropylmethylsulfinyl)furan-2-carboxamide;
N-[3-(5-chloro-1,3-benzoxazol-2-yl)-3-azaspiro[5.5]undecan-9-yl]-2-(trifluoromethyl)pyridine-4-carboxamide;
N-[3-(5-chloro-1,3-benzoxazol-2-yl)-3-azaspiro[5.5]undecan-9-yl]-2-methoxy-pyridine-4-carboxamide;
N-[3-(5-chloro-1,3-benzoxazol-2-yl)-3-azaspiro[5.5]undecan-9-yl]-2-methyl-pyridine-4-carboxamide;
N-[3-(5-chloro-1,3-benzoxazol-2-yl)-3-azaspiro[5.5]undecan-9-yl]-5-sulfamoyl-furan-2-carboxamide;
N-[3-(5-chloro-1,3-benzoxazol-2-yl)-3-azaspiro[5.5]undecan-9-yl]-5-(methylsulfonimidoyl)furan-2-carboxamide;
N-[3-(1,3-benzoxazol-2-yl)-3-azaspiro[5.5]undecan-9-yl]-2-cyclopropylsulfonyl-pyridine-4-carboxamide;
N-[3-(5-chloro-1,3-benzoxazol-2-yl)-3-azaspiro[5.5]undecan-9-yl]-5-(cyclobutanecarbonylsulfamoyl)furan-2-carboxamide;
N-[3-(5-chloro-1,3-benzoxazol-2-yl)-3-azaspiro[5.5]undecan-9-yl]-5-(methylsulfonylmethyl)furan-2-carboxamide;
N-[3-(5-chloro-1,3-benzoxazol-2-yl)-3-azaspiro[5.5]undecan-9-yl]-5-methylsulfonyl-furan-2-carboxamide;
N-[3-(5-chloro-1,3-benzoxazol-2-yl)-3-azaspiro[5.5]undecan-9-yl]-5-(ethylsulfonimidoyl)furan-2-carboxamide;
N-[3-(5-chloro-1,3-benzoxazol-2-yl)-3-azaspiro[5.5]undecan-9-yl]-5-(trifluoromethyl)furan-2-carboxamide;
N-[3-(5-chloro-1,3-benzoxazol-2-yl)-3-azaspiro[5.5]undecan-9-yl]-5-methylsulfinyl-furan-2-carboxamide;
N-[3-(5-chloro-1,3-benzoxazol-2-yl)-3-azaspiro[5.5]undecan-9-yl]-5-ethylsulfonyl-furan-2-carboxamide;
N-[3-(5-chloro-1,3-benzoxazol-2-yl)-3-azaspiro[5.5]undecan-9-yl]-2-cyclopropylsulfonyl-pyridine-4-carboxamide;
N-[3-(5-chloro-1,3-benzoxazol-2-yl)-3-azaspiro[5.5]undecan-9-yl]-5-(cyclopropylmethylsulfonimidoyl)furan-2-carboxamide;
N-[3-(5-chloro-1,3-benzoxazol-2-yl)-3-azaspiro[5.5]undecan-9-yl]-5-(cyclopropylsulfonimidoyl)furan-2-carboxamide;
N-[3-(5-chloro-1,3-benzoxazol-2-yl)-3-azaspiro[5.5]undecan-9-yl]-5-(cyclopropylmethylsulfonyl)furan-2-carboxamide;
N-[3-(5-chloro-1,3-benzoxazol-2-yl)-3-azaspiro[5.5]undecan-9-yl]-5-cyclopropylsulfonyl-furan-2-carboxamide;
N-[2-(5-chloro-1,3-benzoxazol-2-yl)-3,3a,4,5,6,6a-hexahydro-1H-cyclopenta[c]pyrrol-5-yl]-5-(trifluoromethyl)furan-2-carboxamide;
N-[2-(5-chloro-1,3-benzoxazol-2-yl)-3,3a,4,5,6,6a-hexahydro-1H-cyclopenta[c]pyrrol-5-yl]-1,1-dioxo-thiolane-3-carboxamide; and,
N-[3-(5-chloro-1,3-benzoxazol-2-yl)-3-azabicyclo[3.1.0]hexan-6-yl]-5-(trifluoromethyl)furan-2-carboxamide;
or a pharmaceutically acceptable salt thereof.

14. A compound according to claim 1, selected from the group consisting of:
N-[[1-(5-chloro-1,3-benzoxazol-2-yl)-4-piperidyl]methyl]-5-methylsulfonyl-pyridine-3-carboxamide;
N-[2-(5-chloro-1,3-benzoxazol-2-yl)-2-azaspiro[3.3]heptan-6-yl]-2-methoxy-pyridine-4-carboxamide;
N-[2-(5-chloro-1,3-benzoxazol-2-yl)-2-azaspiro[3.3]heptan-6-yl]-5-(isobutylsulfonimidoyl)furan-2-carboxamide;
N-[2-(5-chloro-1,3-benzoxazol-2-yl)-2-azaspiro[3.3]heptan-6-yl]-2-methylsulfonyl-pyridine-4-carboxamide;
N-[2-(5-chloro-1,3-benzoxazol-2-yl)-2-azaspiro[3.3]heptan-6-yl]-2-ethylsulfonyl-pyridine-4-carboxamide;
N-[2-(5-chloro-1,3-benzoxazol-2-yl)-2-azaspiro[3.3]heptan-6-yl]-5-isobutylsulfonyl-furan-2-carboxamide;
N-[2-(5-chloro-1,3-benzoxazol-2-yl)-2-azaspiro[3.3]heptan-6-yl]-2-(trifluoromethyl)pyridine-4-carboxamide;
N-[2-(5-chloro-1,3-benzoxazol-2-yl)-2-azaspiro[3.3]heptan-6-yl]-5-methylsulfonyl-furan-2-carboxamide;
N-[2-(5-chloro-1,3-benzoxazol-2-yl)-2-azaspiro[3.3]heptan-6-yl]-5-(cyclopropylmethylsulfonimidoyl)furan-2-carboxamide;
N-[2-(5-chloro-1,3-benzoxazol-2-yl)-2-azaspiro[3.3]heptan-6-yl]-5-ethylsulfonyl-furan-2-carboxamide;
N-[2-(5-chloro-1,3-benzoxazol-2-yl)-2-azaspiro[3.3]heptan-6-yl]-5-(cyclopropylmethylsulfinyl)furan-2-carboxamide;
N-[2-(5-chloro-1,3-benzoxazol-2-yl)-2-azaspiro[3.3]heptan-6-yl]-5-isobutylsulfinyl-furan-2-carboxamide;
N-[2-(5-chloro-1,3-benzoxazol-2-yl)-2-azaspiro[3.3]heptan-6-yl]-5-(cyclopropylmethylsulfonyl)furan-2-carboxamide;
N-[2-(5-chloro-1,3-benzoxazol-2-yl)-2-azaspiro[3.3]heptan-6-yl]-2-cyclopropylsulfonyl-pyridine-4-carboxamide;
N-[2-(5-chloro-1,3-benzoxazol-2-yl)-6-methyl-2-azaspiro[3.3]heptan-6-yl]-5-methylsulfonyl-furan-2-carboxamide;
N-[7-(5-chloro-1,3-benzoxazol-2-yl)-7-azaspiro[3.5]nonan-2-yl]-1,1-dioxo-thiolane-3-carboxamide;
N-[3-(5-chloro-1,3-benzoxazol-2-yl)-3-azaspiro[5.5]undecan-9-yl]-2,2-dimethyl-propanamide;
N-[3-(5-chloro-1,3-benzoxazol-2-yl)-3-azaspiro[5.5]undecan-9-yl]-3-methylsulfonyl-propanamide;
N-[3-(5-chloro-1,3-benzoxazol-2-yl)-3-azaspiro[5.5]undecan-9-yl]-2-tetrahydrofuran-3-yl-acetamide;
N-[3-(5-chloro-1,3-benzoxazol-2-yl)-3-azaspiro[5.5]undecan-9-yl]tetrahydrofuran-3-carboxamide;
N-[3-(5-chloro-1,3-benzoxazol-2-yl)-3-azaspiro[5.5]undecan-9-yl]-4-methyl-tetrahydrothiopyran-4-carboxamide;
N-[3-(5-chloro-1,3-benzoxazol-2-yl)-3-azaspiro[5.5]undecan-9-yl]-1,1-dioxo-thietane-3-carboxamide;
N-[3-(5-chloro-1,3-benzoxazol-2-yl)-3-azaspiro[5.5]undecan-9-yl]-1,1-dioxo-thiolane-3-carboxamide;
(3S)—N-[3-(5-chloro-1,3-benzoxazol-2-yl)-3-azaspiro[5.5]undecan-9-yl]-1,1-dioxo-thiolane-3-carboxamide;
(3R)—N-[3-(5-chloro-1,3-benzoxazol-2-yl)-3-azaspiro[5.5]undecan-9-yl]-1,1-dioxo-thiolane-3-carboxamide;
N-[3-(5-chloro-1,3-benzoxazol-2-yl)-3-azaspiro[5.5]undecan-9-yl]-3-methyl-1,1-dioxo-thiolane-3-carboxamide;
N-[3-(6-chloro-1,3-benzoxazol-2-yl)-3-azaspiro[5.5]undecan-9-yl]-1,1-dioxo-thiolane-3-carboxamide;
N-[3-(5-chloro-1,3-benzoxazol-2-yl)-3-azaspiro[5.5]undecan-9-yl]-2,2-dioxo-2-λ⁶-thiaspiro[3.3]heptane-6-carboxamide;
N-[3-(5-chloro-1,3-benzoxazol-2-yl)-3-azaspiro[5.5]undecan-9-yl]-1,1-dioxo-thiane-4-carboxamide;
N-[3-(5-chloro-1,3-benzoxazol-2-yl)-3-azaspiro[5.5]undecan-9-yl]-1,1-dioxo-thiane-3-carboxamide;
N-[3-(5-chloro-1,3-benzoxazol-2-yl)-3-azaspiro[5.5]undecan-9-yl]-5-ethylsulfinyl-furan-2-carboxamide;
N-[3-(5-chloro-1,3-benzoxazol-2-yl)-3-azaspiro[5.5]undecan-9-yl]-5-(cyclopropylmethylsulfinyl)furan-2-carboxamide;
N-[3-(5-chloro-1,3-benzoxazol-2-yl)-3-azaspiro[5.5]undecan-9-yl]-2-(trifluoromethyl)pyridine-4-carboxamide;
N-[3-(5-chloro-1,3-benzoxazol-2-yl)-3-azaspiro[5.5]undecan-9-yl]-2-methoxy-pyridine-4-carboxamide;
N-[3-(5-chloro-1,3-benzoxazol-2-yl)-3-azaspiro[5.5]undecan-9-yl]-2-methyl-pyridine-4-carboxamide;
N-[3-(5-chloro-1,3-benzoxazol-2-yl)-3-azaspiro[5.5]undecan-9-yl]-2-cyclopropylsulfonyl-pyridine-4-carboxamide;
N-[3-(5-chloro-1,3-benzoxazol-2-yl)-3-azaspiro[5.5]undecan-9-yl]-5-(cyclopropylmethylsulfonimidoyl)furan-2-carboxamide;
N-[3-(5-chloro-1,3-benzoxazol-2-yl)-3-azaspiro[5.5]undecan-9-yl]-5-(cyclopropylsulfonimidoyl)furan-2-carboxamide;
N-[3-(5-chloro-1,3-benzoxazol-2-yl)-3-azaspiro[5.5]undecan-9-yl]-5-(cyclopropylmethylsulfonyl)furan-2-carboxamide; and,
N-[3-(5-chloro-1,3-benzoxazol-2-yl)-3-azaspiro[5.5]undecan-9-yl]-5-cyclopropylsulfonyl-furan-2-carboxamide;
or a pharmaceutically acceptable salt thereof.

15. A process for the preparation of a compound according to claim 1 comprising the following step,
(a) Coupling of a compound of formula (V), 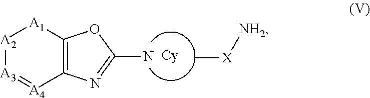
with a compound of formula (VI), 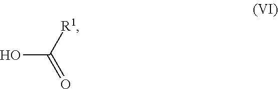
in the presence of a coupling reagent and a base.

16. A compound according to claim 1 for use as therapeutically active substance.

17. A pharmaceutical composition comprising a compound in accordance with claim 1 and a therapeutically inert carrier.

18. The use of a compound according to claim 1 for the treatment of HBV infection.

19. The use of a compound according to claim 1 for the preparation of a medicament for the treatment of HBV infection.

20. The use of a compound according to claim 1 for the inhibition of HBeAg.

21. The use of a compound according to claim 1 for the inhibition of HBsAg.

22. The use of a compound according to claim 1 for the inhibition of HBV DNA.

23. A compound according to claim 1 for use in the treatment of HBV infection.

24. A compound according to claim 1, when manufactured according to a process of claim 15.

25. A method for the treatment of HBV infection, which method comprises administering an effective amount of a compound as defined in claim 1.
